(19) United States
(12) Patent Application Publication
(10) Pub. No.: US 2024/0050546 A1
(43) Pub. Date: Feb. 15, 2024

(54) VARIANT STAPHYLOCOCCUS AUREUS LUKA AND LUKB POLYPEPTIDES AND VACCINE COMPOSITIONS

(71) Applicants: JANSSEN PHARMACEUTICALS, INC., Titusville, NJ (US); NEW YORK UNIVERSITY, New York, NY (US)

(72) Inventors: Brian MORROW, Raritan, NJ (US); Sergey KONSTANTINOV, Leiden (NL); Jeroen GEURTSEN, Leiden (NL); Jinquan LUO, Spring House, PA (US); Sandeep SOMANI, Spring House, PA (US); Peter T. BUCKLEY, Spring House, PA (US); Victor J. TORRES, New York, NY (US)

(21) Appl. No.: 18/028,477

(22) PCT Filed: Sep. 28, 2021
(86) PCT No.: PCT/US2021/052418

§ 371 Date: Mar. 24, 2023

Related U.S. Application Data
(60) Provisional application No. 63/084,273, filed on Sep. 28, 2020.

(51) Int. Cl. A61K 39/085; C07K 14/31; A61P 31/04

(52) U.S. Cl. CPC A61K 39/085; C07K 14/31; A61P 31/04

(57) ABSTRACT

The present disclosure relates to Staphylococcus aureus leukocidin A (LukA) and leukocidin B (LukB) variant polypeptides, and polynucleotides encoding the LukA, LukB and LukAB variant polypeptides. The present disclosure further relates to vaccine compositions comprising these LukA and LukB variants, and methods of generating an immune response against Staphylococcus aureus in a subject.

This application is a U.S. national stage filing under 35 U.S.C. § 371 of International Application No. PCT/US2021/052418, filed Sep. 28, 2021, which claims the priority benefit of U.S. Provisional Patent Application Ser. No. 63/084,273, filed Sep. 28, 2020, which is hereby incorporated by reference in its entirety.

### FIELD

The present disclosure relates to variant Staphylococcus aureus leukocidin A (LukA) and leukocidin B (LukB) proteins and polypeptides, vaccine compositions comprising these LukA and LukB variants, and use of the described vaccine compositions for inducing an immune response in a subject for the treatment and/or prevention of Staphylococcus aureus infection.

### SEQUENCE LISTING

This application contains a computer readable Sequence Listing, which has been submitted electronically in TXT format and is hereby incorporated by reference in its entirety. Said TXT copy, created on Sep. 11, 2023, is named 142772.000194_ST25.txt and is 159,371 bytes in size.

### BACKGROUND

Staphylococcus aureus causes a broad range of invasive diseases, including sepsis, infective endocarditis, and toxic shock, along with less severe skin and soft tissue infections (Tong et al., “Staphylococcus aureus Infections: Epidemiology, Pathophysiology, Clinical Manifestations, and Management,” Clin. Microbiol. Rev. 28(3):603-661 (2015)). Currently, no vaccine is approved to combat S. aureus and therapeutic options are further limited by emerging antibiotic resistance (Sause et al., “Antibody-Based Biologics and Their Promise to Combat Staphylococcus aureus Infections,” Trends Pharmacol. Sci. 37(3):231-241 (2016)). The ability of S. aureus to cause diverse clinical syndromes is often linked to major changes in genome content (Copin et al., “After the Deluge: Mining Staphylococcus aureus Genomic Data for Clinical Associations and Host-Pathogen Interactions,” Curr. Opin. Microbiol. 41:43-50 (2018) and Recker et al., “Clonal Differences in Staphylococcus aureus Bacteraemia-Associated Mortality,” Nat. Microbiol. 2(10):1381-1388 (2017)). Notably, approximately 40% of the genome is not shared by all S. aureus isolates (Bosi et al., “Comparative Genome-Scale Modelling of Staphylococcus aureus Strains Identifies Strain-Specific Metabolic Capabilities Linked to Pathogenicity,” Proc. Natl. Acad. Sci. USA 113(26):E3801-3809 (2016)), thereby further complicating the identification of conserved targets for the generation of vaccines and biologics.

The present disclosure is directed to overcoming these and other limitations in the art.

### SUMMARY

A first aspect of the present disclosure relates to variant Staphylococcus aureus Leukocidin A (LukA) proteins or polypeptides thereof. In one aspect, the LukA variant is a variant of a LukA protein or polypeptide of SEQ ID NO: 25, comprising an amino acid substitution at one or more amino acid residues corresponding to amino acid residues Lys83, Ser141, Val113, Val193 of SEQ ID NO: 25.

Additional aspects of the disclosure relate to LukA variants having one or more additional amino acid substitutions, deletions, and/or additions to those described above. The disclosure also relates to nucleic acid molecules encoding the variant LukA proteins or polypeptides, and expression vectors comprising the aforementioned nucleic acid molecules.

Another aspect of the present disclosure relates to variant Staphylococcus aureus Leukocidin B (LukB) proteins or polypeptides thereof. In one aspect, the LukB variant is a variant of a LukB protein or polypeptide of SEQ ID NO:39 comprising an amino acid substitution at the amino acid residue corresponding to amino acid residue Val53 of SEQ ID NO: 39.

In one aspect, the LukB variant is a variant of a LukB protein or polypeptide of SEQ ID NO: 39 comprising an amino acid substitution at amino acid residues corresponding to amino acid residues Glu45, Glu109, Thr121, and Arg154.

Additional aspects of the disclosure relate to LukB variants having one or more additional amino acid substitutions, deletions, and/or additions to those described above. The disclosure also relates to nucleic acid molecules encoding the variant LukB proteins or polypeptides, and expression vectors comprising the aforementioned nucleic acid molecules.

Another aspect of the present disclosure is directed to an expression vector comprising a nucleic acid molecule encoding a LukA variant polypeptide as described herein operably coupled to a nucleic acid molecule encoding a LukB polypeptide or a LukB variant polypeptide as described herein.

Another aspect of the present disclosure is directed to a host cell comprising any one or more of the expression vectors as described herein.

Another aspect of the present disclosure is directed to a Staphylococcus aureus vaccine composition comprising a LukA variant polypeptide as described herein.

Another aspect of the present disclosure is directed to a Staphylococcus aureus vaccine composition comprising a LukB variant polypeptide as described herein.

Another aspect of the present disclosure is directed to a Staphylococcus aureus vaccine composition comprising a LukA variant polypeptide as described herein and a LukB variant polypeptide as described herein.

Another aspect of the present disclosure relates to a method of generating an immune response against Staphylococcus aureus in a subject. The method involves administering a vaccine composition as described herein to a subject under conditions effective to generate said immune response against S. aureus in said subject.

Staphylococcus aureus (S. aureus) is responsible for a large number of hospital and community acquired infections. To escape clearance by the immune system, S. aureus employs a wide range of strategies, including secretion of bi-component pore-forming toxins known as leukocidins. Each leukocidin is comprised of two polypeptides about 300 amino acids long, grouped as the host cell targeting (S, for slow, based on its chromatographic elution profile) and polymerization (F, for fast) subunits. Up to five leukocidins have been described in human S. aureus isolates: Panton-Valentine Leukocidin (PVL, or LukSF-PV), gamma-hemolysins (HlgAB and HlgCB), leukocidin ED (LukED) and leukocidin AB (LukAB, also referred to as LukGH). Leukocidins bind to specific cell surface protein receptors and assemble into oligomeric pores, eventually leading to cell lysis due to rapid osmotic deregulation (Spaan et al., “Leukocidins: Staphylococcal Bi-Component Pore-Forming Toxins find their Receptors” Nat. Rev. Microbiol. 15(7):435-447(2017)).

The most recently identified leukocidin, LukAB (DuMont et al., “Characterization of a New Cytotoxin that Contributes to Staphylococcus aureus Pathogenesis” Mol. Microbiol. 79(3):814-825 (2011)), possesses several unique features that distinguish it from the other bi-component leukocidins. The LukA (S type) and LukB (F type) subunits exist as a preassembled dimer in solution rather than individual monomers (DuMont et al., “Identification of a Crucial Residue Required for Staphylococcus aureus LukAB Cytotoxicity and Receptor Recognition” Infection and Immunity 82(3):1268-1276 (2014)). Moreover, LukAB targets the CD11b/CD18 integrin on the host cell surface (DuMont et al., “Staphylococcus aureus LukAB cytotoxin kills human neutrophils by targeting the CD11b subunit of the integrin Mac-1” Proc Natl Acad Sci USA. 110(26):10794-9 (2013)), unlike PVL, LukED, HlgAB, and HlgCB, which interact with specific seven-transmembrane chemokine receptors (Spaan et al., “Leukocidins: Staphylococcal Bi-Component Pore-Forming Toxins find their Receptors” Nat. Rev. Microbiol. 15(7):435-447(2017)). Lastly, LukAB is the major toxin responsible for S. aureus-mediated cell lysis in ex vivo infection models using primary human leukocytes (DuMont et al., “Characterization of a New Cytotoxin that Contributes to Staphylococcus aureus Pathogenesis” Mol. Microbiol. 79(3):814-825 (2011)). These unique features are likely explained by the divergence of LukAB from the other leukocidins. Typically, within each group, type S- and F-components share 71-82% and 65-81% identity, respectively, whereas LukA and LukB are only 30% and 39% identical to the other leukocidins. Importantly, leukocidin AB (LukAB) is present in all human infection isolates described to date and it exhibits up to 20% amino acid divergence between the most distant staphylococcal lineages.

Given the importance of the LukAB toxin in S. aureus pathogenesis and infection, novel LukA and LukB variant proteins and polypeptides are disclosed herein. The LukA and LukB variant proteins and polypeptides disclosed herein retain their ability to dimerize to each other, making them ideal vaccine candidates because they maintain and present native toxin structure to the immune system for a robust immune response, but lack cytotoxic activity. Given the large number of individuals who contract S. aureus annually, it is likely that a substantial proportion of these infections will be refractory to traditional courses of antibiotic treatment. The innovative approach to treat, and more importantly prevent, such infections described herein involves inhibiting S. aureus virulence factors, such as LukAB, which are responsible for killing polymorphonuclear leukocytes (PMNs), the most critical innate immune cell involved in defense against S. aureus infection.

### BRIEF DESCRIPTION OF THE DRAWINGS

FIG. 1 is an alignment of fifteen different Staphylococcus aureus LukA amino acid sequences including LukA of clonal complex (CC) 8 (SEQ ID NO: 1); CC45 (SEQ ID NO: 2); HMPREF0772_044(TCH60) of CC30 (SEQ ID NO: 27); SAR2108(MRSA252) of CC30 (SEQ ID NO: 36); SALG_02329(A9635) of CC45 (SEQ ID NO: 34); SAPIG2061(ST398) of CC398 (SEQ ID NO: 35); SATG_01930(D139) of CC10 (SEQ ID NO: 37); NEWMAN of CC8 (SEQ ID NO: 26); SAB1876C(RF122) of CC151 (SEQ ID NO: 32); SAV2005(Mu50) of CC5 (SEQ ID NO: 38); SA1813(N315) of CC5 (SEQ ID NO: 31); SACOL2006 of CC8 (SEQ ID NO: 33); HMPRE0776_0173 USA300(TCH959) of CC7 (SEQ ID NO: 29); HMPREF0774_2356 TCH130 of CC72 (SEQ ID NO: 28); and MW1942 (MW2) of CC1 (SEQ ID NO: 30). The amino acid sequence of a majority LukA sequence generated from a comparison of the aligned is provided as SEQ ID NO: 25. The locations of amino acid substitutions described herein are also identified within each of the LukA sequences.

FIG. 2 is an alignment of fourteen different Staphylococcus aureus LukB amino acid sequences including LukB CC8 (SEQ ID NO: 15); CC45 (SEQ ID NO: 16); A9635 of CC45 (SEQ ID NO: 40); E1410 of CC30 (SEQ ID NO: 43); MRSA252 of CC30 (SEQ ID NO: 45); D139 of CC10 (SEQ ID NO: 42); Mu.50 of CC5 (SEQ ID NO: 46); JKD6008 of CC239 (SEQ ID NO: 44); COL of CC8 (SEQ ID NO: 41); USA300_FPR3757 of CC8 (SEQ ID NO: 50); NEWMAN of CC8 (SEQ ID NO: 51); RF122 of CC151 (SEQ ID NO: 48); MW2 of CC1 (SEQ ID NO: 47); and TCH130 of CC72 (SEQ ID NO: 49). The amino acid sequence of a majority LukB sequence generated from a comparison of the aligned is provided as SEQ ID NO: 39. The locations of amino acid substitutions described herein are also identified within each of the LukB sequences.

FIG. 3 shows the cytotoxicity of different LukAB variants used for immunization. Intoxication of primary human PMNs (n=4) with a titration of the different LukAB variants was carried out for 1 hr. Cell viability was evaluated with CellTiter. Data are the mean±SEM from 4 donors, obtained in 2 separate experiments.

FIGS. 4A-4B show antibody titers against LukAB CC8 or CC45 in mice immunized with different LukAB variants. Envigo Hsd:ND4 (4 week old) mice (n=5/antigen) were subcutaneously administered 20 μg of LukAB in 50 μl of 10% glycerol 1×TBS mixed with 50 μl of the adjuvant, TiterMax® Gold. A cohort of 5 mice also received a mock immunization consisting of an equal volume of 10% glycerol 1×TBS and TiterMax® Gold. Following two boosts (interval of 2 weeks between boosts) of the same antigen adjuvant cocktail, mice were bled via cardiac puncture and serum was obtained. Sera from immunized mice with indicated immunization antigens was pooled and serially diluted to determine antibody titers for CC8 LukAB (FIG. 4A) or CC45 LukAB (FIG. 4B). Plates were coated with 2 g/ml of CC8 or CC45 LukAB. Heatmap shows average absorbance value from duplicate measurements.

FIG. 5 provides the neutralization profile for sera from mice immunized with different LukAB variants against various LukAB toxins. Intoxication of human PMNs (n=4) for 1 hr with 0.156 μg/ml (LD90) of the indicated LukAB variants in the presence of 4-0.031% sera from mice immunized with the indicated antigens. Sera from immunized mice with indicated immunization antigens was pooled and heat inactivated before use. Cell viability was determined with CellTiter. Heatmap displays the average percentage of death cells of 4 donors, with black representing no cell death, and white 100% cell death.

FIGS. 6A-6C are tables showing the percentage of dead human polymorphonuclear leukocytes following intoxication with LD₉₀ of LukAB toxin sequence variants in the absence or presence of 2% (FIG. 6A), 1% (FIG. 6B), and 0.5% (FIG. 6C) mouse sera from mice immunized with the indicated antigen. Data are presented as the percent of dead cells. Cells with no shading represent lowest cell death and cells with darkest grey shading represent highest cell death.

FIGS. 7A-7D show intoxication of LukAB RARPR-33 at high concentrations is not cytotoxic. Freshly isolated human PMNs from healthy donors (n=4-6) were incubated for 1 hour with different concentrations of LukAB variants. Cell viability was determined by absorbance of CellTiter (FIGS. 7A and 7B). Percentage of dead cells was calculated by subtracting background (healthy cells+PBS) and normalizing to Triton X100 treated cells which were set at 100% dead. Mean±SEM is shown. Human PMNs isolated from healthy donors (n=4-6) were incubated for 2 hours with different concentrations of LukAB variants. Cell viability was determined by LDH release. (FIGS. 7C and 7D). Mean±SEM is shown.

FIGS. 8A-8D shows intoxication of LukAB RARPR-33 and the D39A/R23E toxoid at high concentrations. Freshly isolated human PMNs from healthy donors (n=5) were incubated for 2 hours with different concentrations of LukAB variants. Cell viability was determined by absorbance of CellTiter (FIGS. 8A and 8B). Percentage of dead cells was calculated by subtracting background (healthy cells+PBS) and normalizing to Triton X100 treated cells which were set at 100% dead. Mean±SEM is shown. Human PMNs isolated from healthy donors (n=5) were incubated for 2 hours with different concentrations of the LukAB variants. Cell viability was determined by LDH release (FIGS. 8C and 8D). Mean±SEM is shown.

FIG. 9 shows the neutralization profile of sera from mice immunized with two different LukAB toxoids against various LukAB toxins. Intoxication of human PMNs (n=4) for 1 hr with 0.156 μg/ml (LD₉₀) of the indicated LukAB toxin variants in the presence of 0.125% sera from mice immunized with the two different antigens. Sera from mice immunized with both indicated immunization antigens was pooled and heat inactivated before use. Cell viability was determined with CellTiter. Bar graphs show mean+SEM of 4 different donors. Statistical significance was determined using unpaired t test and P<0.05 were considered significant. *P<0.05, **P<0.001, ***P<0.0001.

### DETAILED DESCRIPTION

The present disclosure is directed to variant Staphylococcus aureus leukocidin proteins and polypeptides and compositions comprising these variant proteins and polypeptides. The disclosure further relates to methods for preventing Staphylococcus aureus infections in a subject.

### Definitions

Before the present compositions and methods are described, it is to be understood that this invention is not limited to the particular compositions or methodologies described, as these may vary. It is also to be understood that the terminology used in the description is for the purpose of describing the particular versions or embodiments only, and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present). As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., Staphylococcus LukA and LukB polypeptides and the polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, (1995 Supplement)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information.

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector,” refers to e.g. any number of nucleic acids into which a desired sequence can be inserted, e.g., be restriction and ligation, for transport between genetic environments or for expression in a host cell. Nucleic acid vectors can be DNA or RNA. Vectors include, but are not limited to, plasmids, phage, phagemids, bacterial genomes, virus genomes, self-amplifying RNA, replicons.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptide can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The polypeptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated polypeptide refers to one that can be administered to a subject as an isolated polypeptide; in other words, the polypeptide may not simply be considered “isolated” if it is adhered to a column or embedded in a gel. Moreover, an “isolated nucleic acid fragment” or “isolated peptide” is a nucleic acid or protein fragment that is not naturally occurring as a fragment and/or is not typically in the functional state.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate, or polypeptide of the disclosure in a recipient subject. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response can also involve activation of monocytes, macrophages, NK cells, basophils, dendritic cells, astrocytes, microglia cells, eosinophils, or other components of innate immunity. As used herein “active immunity” refers to any immunity conferred upon a subject by administration of an antigen.

S. aureus Leukocidin A (LukA) Variants

A first aspect of the present disclosure relates to variants, i.e., non-naturally occurring, Staphylococcus aureus (S. aureus) Leukocidin A (LukA) proteins or polypeptides. These variant LukA proteins or polypeptides comprise one or more amino acid residue insertions, substitutions, and/or deletions that render the LukAB bi-component toxin non-cytotoxic, stabilize the LukAB heterodimer, increase the melting temperature, and/or increase solubility. As described herein, these variant LukA proteins and polypeptides are ideal vaccine antigen candidates and can be administered alone or in combination with a Leukocidin B (LukB) wild-type or variant protein or polypeptide. When administered in combination with a LukB protein or polypeptide thereof, the resulting toxoid mimics the structure of S. aureus LukAB toxin, thereby facilitating the generation of a robust immune response against one of the most potent toxins of S. aureus. In any embodiment, the LukA variant polypeptide is a variant of the full-length LukA protein comprising all of the amino acid residues corresponding to a full-length mature LukA protein sequence. As referred to herein, a “mature” leukocidin protein sequence, is a sequence of the leukocidin protein lacking the amino-terminal secretion signal, which typically comprises the first 27-28 amino acid residues on the amino terminus.

In any embodiment, the LukA variant polypeptide is a variant of a less than the full-length mature LukA protein. In any embodiment, the LukA variant polypeptide is at least 100 amino acid residues in length. In any embodiment, the LukA variant polypeptide is at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300 amino acid residues in length.

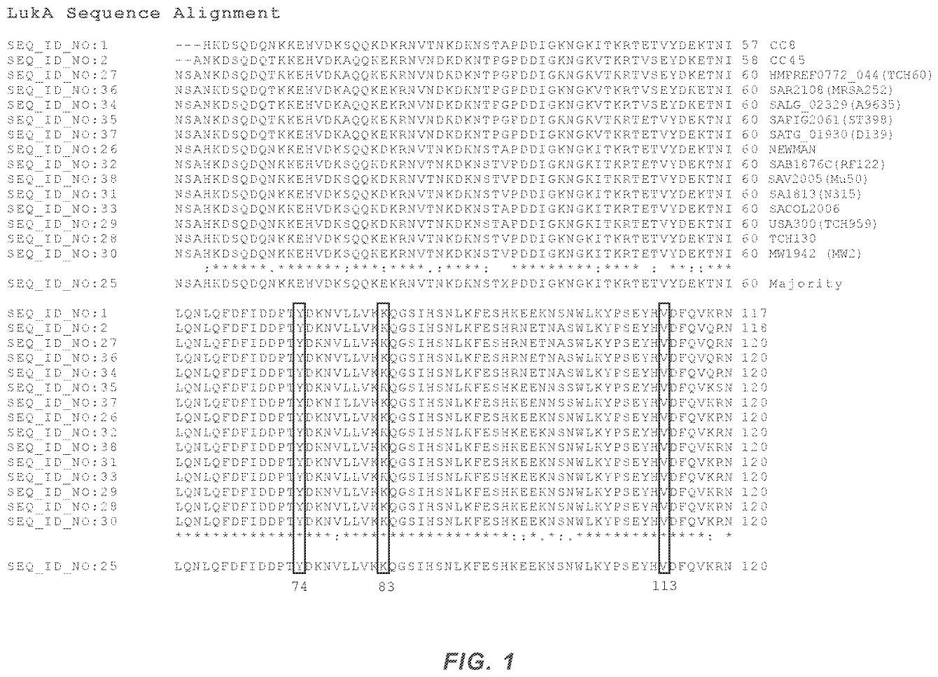

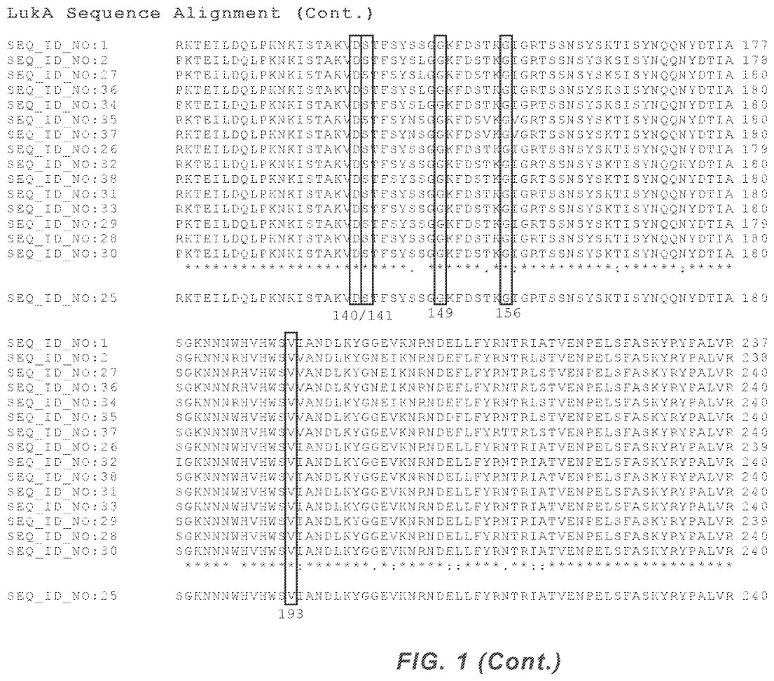

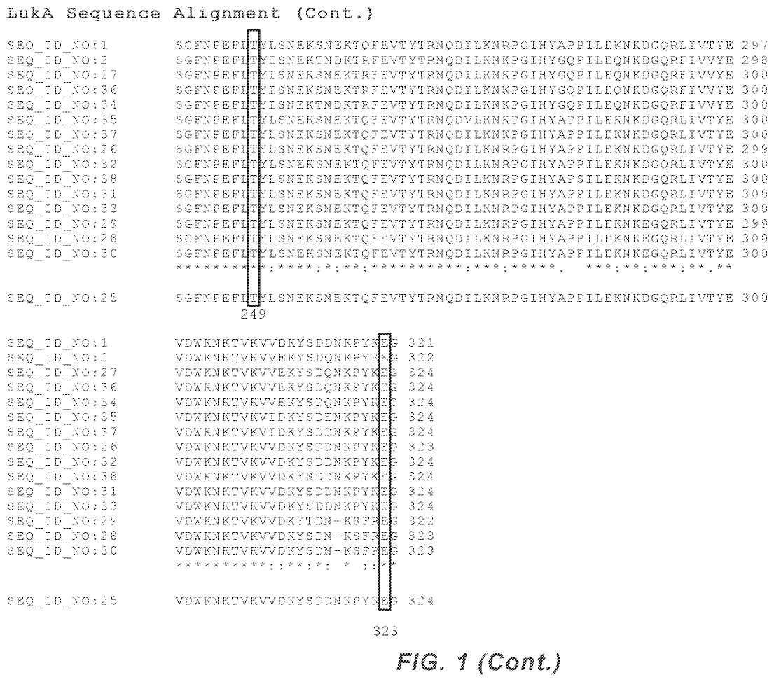

While exemplary LukA variant proteins and polypeptides described herein are variant LukA proteins of clonal complexes CC8 (SEQ ID NO: 1) and CC45 (SEQ ID NO: 2) (see Table 1 below), one of skill in the art will readily appreciate that the amino acid substitutions and/or deletions of LukA identified in the context of SEQ ID NO: 1 and SEQ ID NO: 2 are amino acid residues that are conserved across various clonal complexes or within regions of LukA that are highly conserved across the various clonal complexes. Indeed, an alignment of LukA protein sequences from fifteen different strains of S. aureus (see FIG. 1) shows that the amino acid residues identified herein as residues subject to variation are residues that are conserved across all 15 of the aligned LukA amino acid sequences. While the position of the identified residue of variation may differ between individual LukA sequences, the sequence alignment shows the correspondence between these positions. For clarity, a LukA consensus sequence, having the amino acid sequence of SEQ ID NO: 25, was generated from the sequence alignment and utilized for the purpose of assigning the location of particular amino acid variations. For example, an amino acid substitution at lysine residue 83 in SEQ ID NO: 25 corresponds to the lysine residue at position 80 in the LukA sequence of SEQ ID NO: 1, the lysine residue at position 81 in the LukA sequence of SEQID NO: 2, and the lysine residue at position 83 in the LukA sequences of SEQ ID NOs: 26-38. Thus, the identified amino acid variations described herein can be universally applied to the corresponding amino acid residues of any LukA amino acid sequence known now or in the future.

In accordance with this aspect of the disclosure, in any embodiment, the LukA variant polypeptide comprises an amino acid residue insertion, substitution, and/or deletion at one or more amino acid residues corresponding to residues Lys83, Ser141, Val113, Val193 of SEQ ID NO: 25. In any embodiment, the LukA variant polypeptide further comprises an amino acid substitution or deletion at the amino acid residue corresponding to Glu323 of SEQ ID NO: 25 in addition to the one or more amino acid residue insertions, substitutions, and/or deletions described above. In any embodiment, the amino acid substitution or deletion at Glu323 comprises a glutamic acid to alanine substitution at position 323 (Glu323Ala) of SEQ ID NO: 25.

In any embodiment, the amino acid substitution at the one or more identified positions of LukA (and LukB as described herein) is a conservative substitution. Such conservative substitutions involve substituting one amino acid residue for another that is a member of the same class, which acts as a functional equivalent, resulting in a silent alteration. That is to say, the change relative to the native sequence would not appreciably diminish the basic properties of LukA. These classes of amino acid residues include, nonpolar (hydrophobic) amino acids (e.g., alanine, leucine, isoleucine, valine, proline, phenylalanine, tryptophan and methionine); polar neutral amino acids (e.g., glycine, serine, threonine, cysteine, tyrosine, asparagine, and glutamine); positively charged (basic) amino acids (e.g., arginine, lysine and histidine; and negatively charged (acidic) amino acids (e.g., aspartic acid and glutamic acid).

In other embodiments, an amino acid substitution at the one or more identified positions of the variant leukocidin polypeptide as described herein is a non-conservative alteration (i.e., a substitution that disrupts the sequence, structure, function, or activity of the identified region). Such substitution may be desirable for purposes of reducing or alleviating cytotoxicity of the protein. A non-conservative substitution involves the substitution of an amino acid residue of one particular class with an amino acid residue of a different class. For example, a substitution of a nonpolar (hydrophobic) amino acid residue with a polar neutral amino acid or vice versa. In another embodiment, the non-conservative substitution involves the substitution of a positively charged (basic) amino acid residue, with a negatively charged (acidic) amino acid residue, such as aspartic acid and glutamic acid or vice versa. Such molecular alterations can be accomplished by methods well known in the art, including primer extension on a plasmid template using single stranded templates (Kunkel et al., Proc. Acad. Sci., USA 82:488-492 (1985), which is hereby incorporated by reference in its entirety), double stranded DNA templates (Papworth, et al., Strategies 9(3):3-4 (1996), which is hereby incorporated by reference in its entirety), and by PCR cloning (Braman, J. (ed.), IN VITRO MUTAGENESIS PROTOCOLS, 2nd ed. Humana Press, Totowa, N.J. (2002), which is hereby incorporated by reference in its entirety).

In any embodiment, the LukA variant polypeptide of the present disclosure comprises a lysine to methionine substitution at the residue corresponding to the lysine at position 83 (Lys83Met) of SEQ ID NO: 25. In any embodiment, the LukA variant polypeptide of the present disclosure comprises a lysine to methionine substitution at the residue corresponding to the lysine at position 80 (Lys80Met) of SEQ ID NO: 1. In any embodiment, the LukA variant polypeptide of the present disclosure comprises a lysine to methionine substitution at the residue corresponding to the lysine at position 81 (Lys81Met) of SEQ ID NO: 2.

In any embodiment, the LukA variant polypeptide of the present disclosure comprises a serine to alanine substitution at the residue corresponding to the serine at position 141 (Ser141Ala) of SEQ ID NO: 25. In any embodiment, the LukA variant polypeptide of the present disclosure comprises a serine to alanine substitution at the residue corresponding to the serine at position 138 (Ser138Ala) of SEQ ID NO: 1. In any embodiment, the LukA variant polypeptide of the present disclosure comprises a serine to alanine substitution at the residue corresponding to the serine at position 139 (Ser139Ala) of SEQ ID NO: 2.

In any embodiment, the LukA variant polypeptide of the present disclosure comprises a valine to isoleucine substitution at the residue corresponding to the valine at position 113 (Val113Ile) of SEQ ID NO: 25. In any embodiment, the LukA variant polypeptide of the present disclosure comprises a valine to isoleucine substitution at the residue corresponding to the valine at position 110 (Val110Ile) of SEQ ID NO: 1. In any embodiment, the LukA variant polypeptide of the present disclosure comprises a valine to isoleucine substitution at the residue corresponding to the valine at position 111 (Val111Ile) of SEQ ID NO: 2.

In any embodiment, the LukA variant polypeptide of the present disclosure comprises a valine to isoleucine substitution at the residue corresponding to the valine at position 193 (Val193Ile) of SEQ ID NO: 25. In any embodiment, the LukA variant polypeptide of the present disclosure comprises a valine to isoleucine substitution at the residue corresponding to the valine at position 190 (Val190Ile) of SEQ ID NO: 1. In any embodiment, the LukA variant polypeptide of the present disclosure comprises a valine to isoleucine substitution at the residue corresponding to the valine at position 191 (Val191Ile) of SEQ ID NO: 2.

In any embodiment, the LukA variant polypeptide of the present disclosure comprises a glutamic acid to alanine substitution at the residue corresponding to the glutamic acid residue position 323 (Glu323Ala) of SEQ ID NO: 25 in addition to any one or more of the substitutions at the residues corresponding to Lys83, Ser141, Val113, and Val193 of SEQ ID NO: 25. In any embodiment, the LukA variant polypeptide of the present disclosure comprises a glutamic acid to alanine substitution at the residue corresponding to the glutamic acid residue position 320 (Glu320Ala) of SEQ ID NO: 1 in addition to any one or more of the substitutions at the residues corresponding to Lys80, Ser138, Val110, Val190 of SEQ ID NO: 1. In any embodiment, the LukA variant polypeptide of the present disclosure comprises a glutamic acid to alanine substitution at the residue corresponding to the glutamic acid residue position 321 (Glu321Ala) of SEQ ID NO: 2 in addition to any one or more of the substitutions at the residues corresponding to Lys81, Ser139, Val111, Val191 of SEQ ID NO: 25.

In any embodiment, the LukA variant polypeptide of the present disclosure comprises a polypeptide having an amino acid residue insertion, substitution, and/or deletion at two of the aforementioned amino acid residues corresponding to Lys83, Ser141, Val113, and Val193 of SEQ ID NO: 25. In any embodiment, the LukA variant polypeptide comprises an amino acid residue insertion, substitution, and/or deletion at three of the aforementioned amino acid residues. In any embodiment, the LukA variant polypeptide comprises an amino acid residue insertion, substitution, and/or deletion at all four of the aforementioned amino acid residues. In any embodiment, the LukA variant polypeptide comprises the amino acid substitutions of lysine to methionine, serine to alanine, and valine to isoleucine at the aforementioned amino acid residues corresponding to Lys83Met, Ser141Ala, Val113Ile, and Val193Ile of SEQ ID NO: 25. In any embodiment, the LukA variant polypeptide further comprises the amino acid substitution of glutamic acid to alanine at the amino acid residue corresponding to residue 323 (Glu323Ala) of SEQ ID NO: 25, i.e., the variant LukA comprises substitutions corresponding to Lys83Met, Ser141Ala, Val113Ile, Val193Ile, and Glu323Ala of SEQ ID NO: 25.

An exemplary variant LukA polypeptide of the present disclosure possesses the amino acid substitutions corresponding to Lys83Met, Ser141Ala, Val113Ile, Val193Ile, and Glu323Ala in SEQ ID NO: 25. In any embodiment, the LukA variant polypeptide is CC8 LukA variant comprising amino acid substitutions corresponding to Lys80Met, Ser138Ala, Val110Ile, Val190Ile, and Glu320Ala in SEQ ID NO: 1. In any embodiment, this LukA variant polypeptide has the amino acid sequence of SEQ ID NO: 3, or an amino acid sequence having at least 85%, at least 90%, at least 95%, at least 97%, or at least 99% sequence similarity to the amino acid sequence of SEQ ID NO: 3.

In any embodiment, the LukA variant polypeptide is a CC45 LukA variant polypeptide comprising amino acid substitutions corresponding to Lys81Met, Ser139Ala, Val111Ile, Val191Ile, and Glu321Ala in SEQ ID NO: 2. In any embodiment, this LukA variant polypeptide has the amino acid sequence of SEQ ID NO: 4, or an amino acid sequence having at least 85%, at least 90%, at least 95%, at least 97%, or at least 99% sequence similarity to the amino acid sequence of SEQ ID NO: 4. Other exemplary LukA variant polypeptides include any one of the LukA proteins of SEQ ID NOs: 26-38 comprising the amino acid substitutions corresponding to the substitutions of Lys83Met, Ser141Ala, Val113Ile, Val193Ile, and Glu323Ala in SEQ ID NO: 25.

In any embodiment, the LukA variant polypeptide described herein comprises an amino acid substitution at one or more amino acid residues corresponding to amino acid residues Tyr74, Asp140, Gly149, and Gly156 of SEQ ID NO: 25. In one embodiment, the amino acid substitutions at the one or more aforementioned residues introduces cysteine residues capable of forming disulfide bonds to stabilize conformation of the LukAB heterodimer structure. For example, in one embodiment, the LukA variant polypeptide described herein comprises a tyrosine to cysteine substitution at the amino acid residue corresponding to Tyr74 (Tyr74Cys) of SEQ ID NO: 25, and comprises an asparagine to cysteine substitution at the amino acid residue corresponding to Asp140 (Asp140Cys) of SEQ ID NO: 25. These cysteine residues at positions 74 and 140 form a disulfide bond thereby increasing the thermostability of the variant LukA relative to wild-type LukA or relative to other variant LukA polypeptides not containing paired cysteine residues capable of forming a disulfide bond.

In another embodiment, the LukA variant polypeptide described herein comprises a glycine to cysteine substitution at the amino acid residue corresponding to Gly149 (Gly149Cys) of SEQ ID NO: 25, and comprises a glycine to cysteine substitution at the amino acid residue corresponding to Gly156 (Gly156Cys) of SEQ ID NO: 25. These cysteine residues introduced at positions 149 and 156 form a disulfide bond thereby increasing the thermostability of the variant LukA relative to wild-type LukA or relative to other variant LukA polypeptides not containing paired cysteine residues capable of forming a disulfide bond.

In any embodiment, the LukA variant polypeptide comprises amino acid substitutions at each amino acid residue corresponding to amino acid residues Tyr74, Asp140, Gly149, and Gly156 of SEQ ID NO: 25. In any embodiment, the amino acid substitutions at each of these amino acid residues involves the introduction of a cysteine residue as described above. In any embodiment, the LukA variant polypeptide comprises amino acid substitutions at each amino acid residue corresponding to amino acid residues Tyr71, Asp137, Gly146, and Gly153 of SEQ ID NO: 1. In any embodiment, the amino acid substitutions at each of these amino acid residues involves the introduction of a cysteine residue. In any embodiment, the LukA variant polypeptide comprises amino acid substitutions at each amino acid residue corresponding to amino acid residues Tyr72, Asp138, Gly147, and Gly154 of SEQ ID NO: 2. In any embodiment, the amino acid substitutions at each of these amino acid residues involves the introduction of a cysteine residue.

In any embodiment, the LukA variant polypeptide comprises an amino acid substitution at one or more amino acid residues corresponding to Lys83, Ser141, Val113, Val193, and Glu323 in combination with an amino acid substitution at one or more amino acid residues corresponding to amino acid residues Tyr74, Asp140, Gly149, and Gly156 of SEQ ID NO: 25. In any embodiment, the LukA variant polypeptide comprises amino acid substitutions at amino acid residues corresponding to residues Lys83, Ser141, Val13, Val193, and Glu323 and residues Tyr74, Asp140, Gly149, and Gly156 of SEQ ID NO: 25.

In any embodiment, an exemplary LukA variant polypeptide is a CC8 LukA variant polypeptide having amino acid substitutions at residues corresponding to each of Lys80, Ser138, Val110, Val190, Glu320, Tyr71, Asp137, Gly146, and Gly153 of SEQ ID NO: 1. In any embodiment, an exemplary LukA variant polypeptide is a CC8 LukA variant polypeptide having amino acid substitutions at residues corresponding to each of Lys80Met, Ser138Ala, Val110Ile, Val190Ile, Glu320Ala, Tyr71Cys, Asp137Cys, Gly146Cys, and Gly153Cys of SEQ ID NO: 1. In any embodiment, this CC8 LukA variant polypeptide comprises the amino acid sequence of SEQ ID NO: 5, or an amino acid sequence having at least 85%, at least 90%, at least 95%, at least 97%, or at least 99% sequence similarity to the amino acid sequence of SEQ ID NO: 5.

In any embodiment, an exemplary LukA variant polypeptide is a CC45 LukA variant polypeptide having amino acid substitutions at residues corresponding to each of Lys81, Ser139, Val111, Val191, Glu321, Tyr72, Asp138, Gly147, and Gly154 of SEQ ID NO: 2. In any embodiment, an exemplary LukA variant polypeptide is a CC45 LukA variant polypeptide having amino acid substitutions at residues corresponding to each of Lys81Met, Ser139Ala, Val111Ile, Val191Ile, Glu321Ala, Tyr72Cys, Asp138Cys, Gly147Cys, and Gly154Cys of SEQ ID NO: 2. In any embodiment, this CC45 LukA variant polypeptide comprises the amino acid sequence of SEQ ID NO: 6, or an amino acid sequence having at least 85%, at least 90%, at least 95%, at least 97%, or at least 99% sequence similarity to the amino acid sequence of SEQ ID NO: 6.

Other exemplary LukA variant polypeptides include any one of the LukA proteins of SEQ ID NOs: 26-38 comprising the amino acid substitutions corresponding to Lys83Met, Ser141Ala, Val113Ile, Val193Ile, Glu323, Tyr74, Asp140, Gly149, and Gly156 of SEQ ID NO: 25.

In any embodiment, the LukA variant polypeptide described herein comprises an amino acid substitution or deletion at the amino acid residue corresponding to amino acid residue Thr249 of SEQ ID NO: 25. In any embodiment, the LukA variant comprises a substitution at the residue corresponding to Thr249, where the substitution is a threonine to valine substitution at this residue (Thr249Val). In any embodiment, the LukA variant polypeptide described herein comprises an amino acid substitution or deletion at the amino acid residue corresponding to amino acid residue Thr246 of SEQ ID NO: 1. In any embodiment, the LukA variant polypeptide described herein comprises an amino acid substitution or deletion at the amino acid residue corresponding to amino acid residue Thr247 of SEQ ID NO: 2.

In any embodiment, the LukA variant polypeptide described herein comprises the amino acid substitution at amino acid residue corresponding to Thr249 of SEQ ID NO: 25 in combination with any one of the other amino acid residue substitutions described herein, i.e., substitutions at residues corresponding to Lys83, Ser141, Val113, Val193, Glu323 Tyr74, Asp140, Gly149, and Gly156 of SEQ ID NO: 25. In any embodiment, the LukA variant polypeptide described herein comprises an amino acid substitution at the amino acid residue corresponding to Thr249 of SEQ ID NO: 25 in combination with at least two, at least three, at least four, at least five, at least six, at least seven, at least eight, or all nine of the other amino acid residue substitutions described herein. In any embodiment, the LukA variant polypeptide comprises amino acid substitutions at each residue corresponding to Lys83, Ser14, Val113, Val193, Glu323, and Thr249 of SEQ ID NO: 25.

In any embodiment, an exemplary LukA variant polypeptide is a CC8 LukA variant polypeptide having amino acid substitutions at residues corresponding to each of Lys80, Ser138, Val110, Val190, Glu320, and Thr246 of SEQ ID NO: 1. In any embodiment, an exemplary LukA variant polypeptide is a CC8 LukA variant polypeptide having amino acid substitutions at residues corresponding to each of Lys80Met, Ser138Ala, Val110Ile, Val190Ile, Glu320Ala, and Thr246Val of SEQ ID NO: 1. In one embodiment, an exemplary LukA variant polypeptide having amino acid substitutions at residues corresponding to each of the aforementioned positions has an amino acid sequence of SEQ ID NO: 7, or an amino acid sequence having at least 85%, at least 90%, at least 95%, at least 97%, or at least 99% sequence similarity to the amino acid sequence of SEQ ID NO: 7.

In any embodiment, an exemplary LukA variant polypeptide is a CC45 LukA variant polypeptide having amino acid substitutions at residues corresponding to each of Lys81, Ser139, Val111, Val191, Glu321, and Thr247 of SEQ ID NO: 2. In any embodiment, an exemplary LukA variant polypeptide is a CC45 LukA variant polypeptide having amino acid substitutions at residues corresponding to each of Lys81Met, Ser139Ala, Val111Ile, Val191Ile, Glu321Ala, and Thr247Val of SEQ ID NO: 2. In one embodiment, an exemplary LukA variant polypeptide having amino acid substitutions at residues corresponding to each of the aforementioned positions has an amino acid sequence of SEQ ID NO: 8, or an amino acid sequence having at least 85%, at least 90%, at least 95%, at least 97%, or at least 99% sequence similarity to the amino acid sequence of SEQ ID NO: 8.

Other exemplary LukA variant polypeptides include any one of the LukA proteins of SEQ ID NOs: 26-38 comprising the described amino acid substitutions at the amino acid residues corresponding to Lys83, Ser141, Val113, Val193, Glu323, and Thr249 of SEQ ID NO:25.

In any embodiment, the LukA variant polypeptide comprises amino acid substitutions at each residue corresponding to Lys83, Ser141, Val13, Val193, Glu323, Thr249, Tyr74, Asp140, Gly149, and Gly156 of SEQ ID NO: 25.

In any embodiment, an exemplary LukA variant polypeptide is a CC8 LukA variant polypeptide having amino acid substitutions at residues corresponding to each of Lys80, Ser138, Val110, Val190, Glu320, Tyr71, Asp137, Gly146, Gly153, and Thr246 of SEQ ID NO: 1. In any embodiment, an exemplary LukA variant polypeptide is a CC8 LukA variant polypeptide having amino acid substitutions at residues corresponding to each of Lys80Met, Ser138Ala, Val110Ile, Val190Ile, Glu320Ala, Tyr71Cys, Asp137Cys, Gly146Cys, Gly153Cys, and Thr246Val of SEQ ID NO: 1. In one embodiment, an exemplary LukA variant polypeptide having amino acid substitutions at residues corresponding to each of the aforementioned positions has an amino acid sequence of SEQ ID NO: 9, or an amino acid sequence having at least 85%, at least 90%, at least 95%, at least 97%, or at least 99% sequence similarity to the amino acid sequence of SEQ ID NO: 9.

In any embodiment, an exemplary LukA variant polypeptide is a CC45 LukA variant polypeptide having amino acid substitutions at residues corresponding to each of Lys81, Ser139, Val111, Val191, Glu321, Tyr72, Asp138, Gly147, Gly154 and Thr247 of SEQ ID NO: 2. In any embodiment, an exemplary LukA variant polypeptide is a CC45 LukA variant polypeptide having amino acid substitutions at residues corresponding to each of Lys81Met, Ser139Ala, Val111Ile, Val191Ile, Glu321Ala, Tyr72Cys, Asp138Cys, Gly147Cys, Gly154Cys and Thr247Ala of SEQ ID NO: 2. In one embodiment, an exemplary LukA variant polypeptide having amino acid substitutions at residues corresponding to each of the aforementioned positions has an amino acid sequence of SEQ ID NO: 10, or an amino acid sequence having at least 85%, at least 90%, at least 95%, at least 97%, or at least 99% sequence similarity to the amino acid sequence of SEQ ID NO: 10.

Other exemplary LukA variant polypeptides include any one of the LukA proteins of SEQ ID NOs: 26-38 comprising the described amino acid substitutions of residues corresponding to Lys83, Ser141, Val113, Val193, Glu323, Thr249, Tyr74, Asp140, Gly149, and Gly156 of SEQ ID NO: 25.

Table 1 below provides exemplary variant LukA amino acid sequences as disclosed herein.

TABLE 1
Exemplary LukA Polypeptide Amino Acid Sequences

| SEQ | | |
|---|---|---|
| ID NO | Name | Description |
| 1 | LukA CC8 WT | HKDSQDQNKKEHVDKSQQKDKRNVTNKDKNSTAPDDIG |
| | | KNGKITKRTETVYDEKTNILQNLQFDFIDDPTYDKNVLLV |
| | | KKQGSIHSNLKFESHKEEKNSNWLKYPSEYHVDFQVKRN |
| | | RKTEILDQLPKNKISTAKVDSTFSYSSGGKFDSTKGIGRTS |
| | | SNSYSKTISYNQQNYDTIASGKNNNWHVHWSVIANDLKY |
| | | GGEVKNRNDELLFYRNTRIATVENPELSFASKYRYPALVR |
| | | SGFNPEFLTYLSNEKSNEKTQFEVTYTRNQDILKNRPGIHY |
| | | APPILEKNKDGQRLIVTYEVDWKNKTVKVVDKYSDDNKP |
| | | YKEG |
| 2 | LukA CC45 WT | ANKDSQDQTKKEHVDKAQQKEKRNVNDKDKNTPGPDDI |
| | | GKNGKVTKRTVSEYDKETNILQNLQFDFIDDPTYDKNVL |
| | | LVKKQGSIHSNLKFESHRNETNASWLKYPSEYHVDFQVQ |
| | | RNPKTEILDQLPKNKISTAKVDSTFSYSLGGKFDSTKGIGR |
| | | TSSNSYSKSISYNQQNYDTIASGKNNNRHVHWSVVANDL |
| | | KYGNEIKNRNDEFLFYRNTRLSTVENPELSFASKYRYPAL |
| | | VRSGFNPEFLTYISNEKTNDKTRFEVTYTRNQDILKNKPGI |
| | | HYGQPILEQNKDGQRFIVVYEVDWKNKTVKVVEKYSDQ |
| | | NKPYKEG |
| 3 | LukA CC8 W95 | HKDSQDQNKKEHVDKSQQKDKRNVTNKDKNSTAPDDIG |
| | E320A, Lys80Met, | KNGKITKRTETVYDEKTNILQNLQFDFIDDPTYDKNVLLV |
| | Ser138Ala, Val110Ile, | KMQGSIHSNLKFESHKEEKNSNWLKYPSEYHIDFQVKRN |
| | Val190Ile | RKTEILDQLPKNKISTAKVDATFSYSSGGKFDSTKGIGRTS |
| | | SNSYSKTISYNQQNYDTIASGKNNNWHVHWSIIANDLKY |
| | | GGEVKNRNDELLFYRNTRIATVENPELSFASKYRYPALVR |
| | | SGFNPEFLTYLSNEKSNEKTQFEVTYTRNQDILKNRPGIHY |
| | | APPILEKNKDGQRLIVTYEVDWKNKTVKVVDKYSDDNKP |
| | | YKAG |
| 4 | LukA CC45 W95 | ANKDSQDQTKKEHVDKAQQKEKRNVNDKDKNTPGPDDI |
| | E321A, Lys81Met, | GKNGKVTKRTVSEYDKETNILQNLQFDFIDDPTYDKNVL |
| | Ser139Ala, Val111Ile, | LVKMQGSIHSNLKFESHRNETNASWLKYPSEYHIDFQVQ |
| | Val191Ile | RNPKTEILDQLPKNKISTAKVDATFSYSLGGKFDSTKGIGR |
| | | TSSNSYSKSISYNQQNYDTIASGKNNNRHVHWSIVANDL |
| | | KYGNEIKNRNDEFLFYRNTRLSTVENPELSFASKYRYPAL |
| | | VRSGFNPEFLTYISNEKTNDKTRFEVTYTRNQDILKNKPGI |
| | | HYGQPILEQNKDGQRFIVVYEVDWKNKTVKVVEKYSDQ |
| | | NKPYKAG |
| 5 | LukA CC8 W95W72 | HKDSQDQNKKEHVDKSQQKDKRNVTNKDKNSTAPDDIG |
| | E320A, Lys80Met, | KNGKITKRTETVYDEKTNILQNLQFDFIDDPTCDKNVLLV |
| | Ser138Ala, Val110Ile, | KMQGSIHSNLKFESHKEEKNSNWLKYPSEYHIDFQVKRN |
| | Val190Ile, Tyr71Cys, | RKTEILDQLPKNKISTAKVCATFSYSSGCKFDSTKCIGRTS |
| | Asp137Cys, | SNSYSKTISYNQQNYDTIASGKNNNWHVHWSIIANDLKY |
| | Gly146Cys, Gly153Cys | GGEVKNRNDELLFYRNTRIATVENPELSFASKYRYPALVR |
| | | SGFNPEFLTYLSNEKSNEKTQFEVTYTRNQDILKNRPGIHY |
| | | APPILEKNKDGQRLIVTYEVDWKNKTVKVVDKYSDDNKP |
| | | YKAG |
| 6 | LukA CC45 W95W72 | ANKDSQDQTKKEHVDKAQQKEKRNVNDKDKNTPGPDDI |
| | E321A, Lys81Met, | GKNGKVTKRTVSEYDKETNILQNLQFDFIDDPTCDKNVL |
| | Ser139Ala, Val111Ile, | LVKMQGSIHSNLKFESHRNETNASWLKYPSEYHIDFQVQ |
| | Val191Ile, Tyr72Cys, | RNPKTEILDQLPKNKISTAKVCATFSYSLGCKFDSTKCIGR |
| | Asp138Cys, | TSSNSYSKSISYNQQNYDTIASGKNNNRHVHWSIVANDL |
| | Gly147Cys, Gly154Cys | KYGNEIKNRNDEFLFYRNTRLSTVENPELSFASKYRYPAL |
| | | VRSGFNPEFLTYISNEKTNDKTRFEVTYTRNQDILKNKPGI |
| | | HYGQPILEQNKDGQRFIVVYEVDWKNKTVKVVEKYSDQ |
| | | NKPYKAG |
| 7 | LukA CC8 W97 | HKDSQDQNKKEHVDKSQQKDKRNVTNKDKNSTAPDDIG |
| | E320A, Lys80Met, | KNGKITKRTETVYDEKTNILQNLQFDFIDDPTYDKNVLLV |
| | Ser138Ala, Val110Ile, | KMQGSIHSNLKFESHKEEKNSNWLKYPSEYHIDFQVKRN |
| | Val190Ile, Thr246Val | RKTEILDQLPKNKISTAKVNATFSYSSGGKFDSTKGIGRTS |
| | | SNSYSKTISYNQQNYDTIASGKNNNWHVHWSIIANDLKY |
| | | GGEVKNRNDELLFYRNTRIATVENPELSFASKYRYPALVR |
| | | SGFNPEFLVYLSNEKSNEKTQFEVTYTRNQDILKNRPGIH |
| | | YAPPILEKNKDGQRLIVTYEVDWKNKTVKVVDKYSDDN |
| | | KPYKAG |
| 8 | LukA CC45 W97 | ANKDSQDQTKKEHVDKAQQKEKRNVNDKDKNTPGPDDI |
| | E321A, Lys81Met, | GKNGKVTKRTVSEYDKETNILQNLQFDFIDDPTYDKNVL |
| | Ser139Ala, Val111Ile, | LVKMQGSIHSNLKFESHRNETNASWLKYPSEYHIDFQVQ |
| | Val191Ile, Thr247Val | RNPKTEILDQLPKNKISTAKVDATFSYSLGGKFDSTKGIGR |
| | | TSSNSYSKSISYNQQNYDTIASGKNNNRHVHWSIVANDL |
| | | KYGNEIKNRNDEFLFYRNTRLSTVENPELSFASKYRYPAL |
| | | VRSGFNPEFLVYISNEKTNDKTRFEVTYTRNQDILKNKPGI |
| | | HYGQPILEQNKDGQRFIVVYEVDWKNKTVKVVEKYSDQ |
| | | NKPYKAG |
| 9 | LukA CC8 W97 W72 | HKDSQDQNKKEHVDKSQQKDKRNVTNKDKNSTAPDDIG |
| | E320A, Lys80Met, | KNGKITKRTETVYDEKTNILQNLQFDFIDDPTCDKNVLLV |
| | Ser138Ala, Val110Ile, | KMQGSIHSNLKFESHKEEKNSNWLKYPSEYHIDFQVKRN |
| | Val190Ile, Thr246Val, | RKTEILDQLPKNKISTAKVCATFSYSSGCKFDSTKCIGRTS |
| | Tyr71Cys, Asp137Cys, | SNSYSKTISYNQQNYDTIASGKNNNWHVHWSIIANDLKY |
| | Gly146Cys, Gly153Cys | GGEVKNRNDELLFYRNTRIATVENPELSFASKYRYPALVR |
| | | SGFNPEFLVYLSNEKSNEKTQFEVTYTRNQDILKNRPGIH |
| | | YAPPILEKNKDGQRLIVTYEVDWKNKTVKVVDKYSDDN |
| | | KPYKAG |
| 10 | LukA CC45 W97 W72 | ANKDSQDQTKKEHVDKAQQKEKRNVNDKDKNTPGPDDI |
| | E321A, Lys81Met, | GKNGKVTKRTVSEYDKETNILQNLQFDFIDDPTCDKNVL |
| | Ser139Ala, Val111Ile, | LVKMQGSIHSNLKFESHRNETNASWLKYPSEYHIDFQVQ |
| | Val191Ile, Thr247Val, | RNPKTEILDQLPKNKISTAKVCATFSYSLGCKFDSTKCIGR |
| | Tyr72Cys, Asp138Cys, | TSSNSYSKSISYNQQNYDTIASGKNNNRHVHWSIVANDL |
| | Gly147Cys, Gly154Cys | KYGNEIKNRNDEFLFYRNTRLSTVENPELSFASKYRYPAL |
| | | VRSGFNPEFLVYISNEKTNDKTRFEVTYTRNQDILKNKPGI |
| | | HYGQPILEQNKDGQRFIVVYEVDWKNKTVKVVEKYSDQ |
| | | NKPYKAG |
| 11 | LukA CC45 W94 | ANKDSQDQTKKEHVDKAQQKEKRNVNDKDKNTPGPDDI |
| | E321A, Lys81Leu, | GKNGKVTKRTVSEYDKETNILQNLQFDFIDDPTYDKNVL |
| | Ser139Ala, Val111Ile, | LVKLQGSIHSNLKFESHRNETNASWLKYPSEYHIDFQVQR |
| | Val191Ile | NPKTEILDQLPKNKISTAKVDATFSYSLGGKFDSTKGIGRT |
| | | SSNSYSKSISYNQQNYDTIASGKNNNRHVHWSIVANDLK |
| | | YGNEIKNRNDEFLFYRNTRLSTVENPELSFASKYRYPALV |
| | | RSGFNPEFLTYISNEKTNDKTRFEVTYTRNQDILKNKPGIH |
| | | YGQPILEQNKDGQRFIVVYEVDWKNKTVKVVEKYSDQN |
| | | KPYKAG |
| 12 | LukA CC45 W96 | ANKDSQDQTKKEHVDKAQQKEKRNVNDKDKNTPGPDDI |
| | E321A, Lys81Leu, | GKNGKVTKRTVSEYDKETNILQNLQFDFIDDPTYDKNVL |
| | Ser139Ala, Val111Ile, | LVKLQGSIHSNLKFESHRNETNASWLKYPSEYHIDFQVQR |
| | Val191Ile, Thr247Val | NPKTEILDQLPKNKISTAKVDATFSYSLGGKFDSTKGIGRT |
| | | SSNSYSKSISYNQQNYDTIASGKNNNRHVHWSIVANDLK |
| | | YGNEIKNRNDEFLFYRNTRLSTVENPELSFASKYRYPALV |
| | | RSGFNPEFLVYISNEKTNDKTRFEVTYTRNQDILKNKPGIH |
| | | YGQPILEQNKDGQRFIVVYEVDWKNKTVKVVEKYSDON |
| | | KPYKAG |
| 13 | LukA CC8 | HKDSQDQNKKEHVDKSQQKDKRNVTNKDKNSTAPDDIG |
| | Glu320Ala | KNGKITKRTETVYDEKTNILQNLQFDFIDDPTYDKNVLLV |
| | | KKQGSIHSNLKFESHKEEKNSNWLKYPSEYHVDFQVKRN |
| | | RKTEILDQLPKNKISTAKVDSTFSYSSGGKFDSTKGIGRTS |
| | | SNSYSKTISYNQQNYDTIASGKNNNWHVHWSVIANDLKY |
| | | GGEVKNRNDELLFYRNTRIATVENPELSFASKYRYPALVR |
| | | SGFNPEFLTYLSNEKSNEKTQFEVTYTRNQDILKNRPGIHY |
| | | APPILEKNKDGQRLIVTYEVDWKNKTVKVVDKYSDDNKP |
| | | YKAG |
| 14 | LukA CC45 Glu321Ala | ANKDSQDQTKKEHVDKAQQKEKRNVNDKDKNTPGPDDI |
| | | GKNGKVTKRTVSEYDKETNILQNLQFDFIDDPTYDKNVL |
| | | LVKKQGSIHSNLKFESHRNETNASWLKYPSEYHVDFQVQ |
| | | RNPKTEILDQLPKNKISTAKVDSTFSYSLGGKFDSTKGIGR |
| | | TSSNSYSKSISYNQQNYDTIASGKNNNRHVHWSVVANDL |
| | | KYGNEIKNRNDEFLFYRNTRLSTVENPELSFASKYRYPAL |
| | | VRSGFNPEFLTYISNEKTNDKTRFEVTYTRNQDILKNKPGI |
| | | HYGQPILEQNKDGQRFIVVYEVDWKNKTVKVVEKYSDQ |
| | | NKPYKAG |
| 64 | LukA CC8 delta 10 | HKDSQDQNKKEHVDKSQQKDKRNVTNKDKNSTAPDDIG |
| | | KNGKITKRTETVYDEKTNILQNLQFDFIDDPTYDKNVLLV |
| | | KKQGSIHSNLKFESHKEEKNSNWLKYPSEYHVDFQVKRN |
| | | RKTEILDQLPKNKISTAKVDSTFSYSSGGKFDSTKGIGRTS |
| | | SNSYSKTISYNQQNYDTIASGKNNNWHVHWSVIANDLKY |
| | | GGEVKNRNDELLFYRNTRIATVENPELSFASKYRYPALVR |
| | | SGFNPEFLTYLSNEKSNEKTQFEVTYTRNQDILKNRPGIHY |
| | | APPILEKNKDGQRLIVTYEVDWKNKTVKVVDKY |
| 65 | LukA CC45 delta 10 | ANKDSQDQTKKEHVDKAQQKEKRNVNDKDKNTPGPDDI |
| | | GKNGKVTKRTVSEYDKETNILQNLQFDFIDDPTYDKNVL |
| | | LVKKQGSIHSNLKFESHRNETNASWLKYPSEYHVDFQVQ |
| | | RNPKTEILDQLPKNKISTAKVDSTFSYSLGGKFDSTKGIGR |
| | | TSSNSYSKSISYNQQNYDTIASGKNNNRHVHWSVVANDL |
| | | KYGNEIKNRNDEFLFYRNTRLSTVENPELSFASKYRYPAL |
| | | VRSGFNPEFLTYISNEKTNDKTRFEVTYTRNQDILKNKPGI |
| | | HYGQPILEQNKDGQRFIVVYEVDWKNKTVKVVEKY |

S. aureus Leukocidin B (LukB) Variants

Another aspect of the present disclosure is directed to S. aureus Leukocidin B (LukB) variant polypeptides. These LukB variant polypeptides comprise one or more amino acid residue insertions, substitutions, and/or deletions that improve LukB stability thereby contributing to LukAB toxoid stability. As described herein, these LukB variant polypeptides are ideal vaccine antigen candidates which can be administered alone or in combination with a Leukocidin A (LukA) wild-type or variant protein or polypeptide. When administered in combination with a LukA wild-type or variant polypeptide, the resulting toxoid mimics the structure of S. aureus LukAB toxin, thereby facilitating the generation of a robust immune response against one of the most potent toxins of S. aureus. In any embodiment, the LukB variant polypeptide is a variant of the full-length LukB protein comprising all of the amino acid residues corresponding to a full-length mature LukB protein sequence. In any embodiment, the LukB variant comprises an amino acid chain of the referenced protein that is less than the full-length mature LukB protein. In one embodiment, the LukB variant polypeptide is at least 100 amino acid residues in length. In any embodiment, the LukB variant polypeptide is at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300 amino acid residues in length.

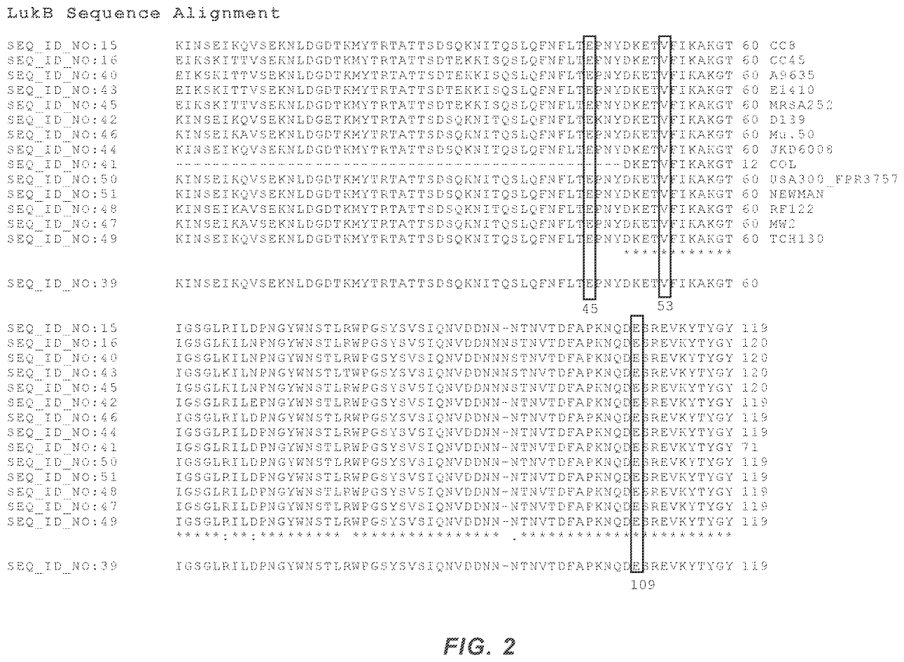

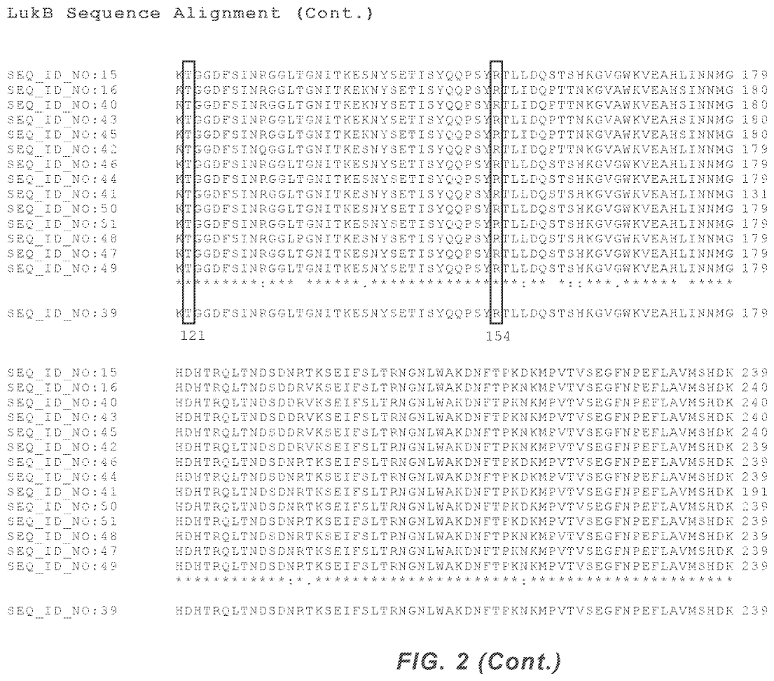

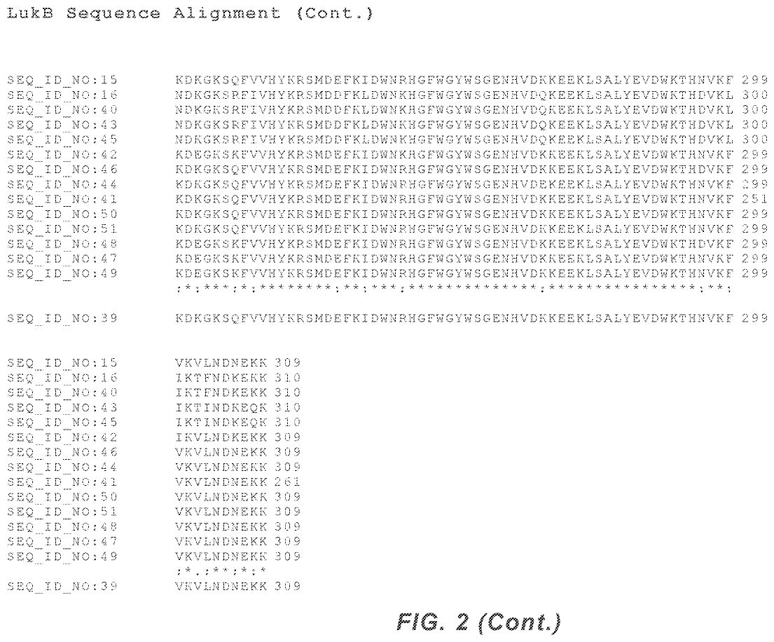

While exemplary LukB variant polypeptides described herein are LukB variants of clonal complexes CC8 (SEQ ID NO: 15) and CC45 (SEQ ID NO: 16) (see Table 2 below), one of skill in the art readily appreciates that the amino acid substitutions and/or deletions of LukB identified in the context of SEQ ID NO: 15 and SEQ ID NO: 16 are amino acid residues that are conserved across various clonal complexes or within regions of LukB that are highly conserved across the various clonal complexes. An alignment of LukB protein sequences from fourteen different strains of S. aureus (see FIG. 2) shows that the amino acid residues identified herein as residues subject to variation are residues that are conserved across all 14 of the aligned LukB amino acid sequences. While the position of the identified residue of variation may differ between individual LukB sequences, the sequence alignment shows the correspondence between these positions. For clarity, a LukB consensus sequence, having the amino acid sequence of SEQ ID NO: 39, was generated from the sequence alignment and utilized for the purpose of assigning the location of particular amino acid variations. For example, an amino acid substitution at glutamic acid residue 109 in SEQ ID NO: 39 corresponds to the glutamic acid residue at position 109 in the LukB sequences of SEQ ID NOs: 15, 42, 44, and 46-51, the glutamic acid residue at position 110 in the LukB sequences of SEQID NOs: 16, 40, 43, and 45, and the glutamic acid residue at position 60 in the LukB sequence of SEQ ID NO: 41. Thus, the identified amino acid variations described herein can be universally applied to corresponding amino acid residues in any LukB amino acid sequence known now or in the future.

In any embodiment, the LukB variant polypeptide as disclosed herein comprises an amino acid substitution or deletion at the amino acid residue corresponding to amino acid residue Val53 of SEQ ID NO: 39. In any embodiment, the amino acid substitution at Val53 comprises a valine to leucine (Val53Leu) substitution. In one embodiment, an exemplary LukB variant polypeptide comprising a substitution corresponding to the Val53Leu substitution in SEQ ID NO: 39.

In any embodiment, an exemplary LukB variant polypeptide is a CC8 LukB variant polypeptide having an amino acid substitution at the amino acid position corresponding to position 53 of SEQ ID NO: 15. In any embodiment, an exemplary LukB variant polypeptide is a CC8 LukB variant polypeptide having a valine to leucine amino acid substitution at the position corresponding to position 53 of SEQ ID NO: 15. In any embodiment, an exemplary CC8 LukB sequence having a valine to leucine substitution at position 53 comprises the amino acid sequence of SEQ ID NO: 17, or an amino acid sequence having at least 85%, at least 90%, at least 95%, at least 97%, or at least 99% sequence similarity to the amino acid sequence of SEQ ID NO: 17.

In any embodiment, an exemplary LukB variant polypeptide is a CC45 LukB variant polypeptide having an amino acid substitution at the amino acid position corresponding to position 53 of SEQ ID NO: 16. In any embodiment, an exemplary LukB variant polypeptide is a CC45 LukB variant polypeptide having a valine to leucine amino acid substitution at the position corresponding to position 53 of SEQ ID NO: 16. In one embodiment, an exemplary LukB variant polypeptide comprising a substitution corresponding to the Val53Leu substitution in SEQ ID NO: 39 comprises the amino acid sequence of SEQ ID NO: 18, or an amino acid sequence having at least 85%, at least 90%, at least 95%, at least 97%, or at least 99% sequence similarity to the amino acid sequence of SEQ ID NO: 18.

Other exemplary LukB variant polypeptides include any one of the LukB proteins of SEQ ID NOs: 40-51 comprising an amino acid substitution corresponding to Val53Leu.

In any embodiment, the LukB variant polypeptide described herein comprises an amino acid substitution at one or more amino acid residues corresponding to amino acid residues Glu45, Glu109, Thr121, and Arg154 of SEQ ID NO: 39. In one embodiment, the amino acid substitution at the one or more aforementioned residues introduces cysteine residues capable of forming a disulfide bond to stabilize conformation of the LukAB heterodimer structure. For example, in one embodiment, the LukB variant protein or polypeptide described herein comprises a glutamic acid to cysteine substitution at the amino acid residue corresponding to Glu45 (Glu45Cys) of SEQ ID NO: 39, and comprises an threonine to cysteine substitution at the amino acid residue corresponding to Thr121 (Thr121Cys) of SEQ ID NO: 39. These cysteine residues at positions 45 and 121 form a disulfide bond thereby increasing the thermostability of the variant LukB relative to wild-type LukB or relative to other variant LukB proteins and polypeptides described herein not containing paired cysteine residues capable of forming a disulfide bond.

In another embodiment, the LukB variant protein or polypeptide described herein comprises a glutamic acid to cysteine substitution at the amino acid residue corresponding to Glu109 (Glu109Cys) of SEQ ID NO: 39, and comprises an arginine to cysteine substitution at the amino acid residue corresponding to Arg154 (Arg154Cys) of SEQ ID NO:39. These cysteine residues introduced at positions 109 and 154 form a disulfide bond thereby increasing the thermostability of the LukB variant relative to wild-type LukB or relative to other LukB variant polypeptides not containing paired cysteine residues capable of forming disulfide bonds.

In any embodiment, the LukB variant polypeptide comprises an amino acid substitution at each amino acid residue corresponding to amino acid residues Glu45, Glu109, Thr121, and Arg154 of SEQ ID NO: 39. In any embodiment, the amino acid substitutions at each of these amino acid residues involves the introduction of a cysteine residue as described above.

In any embodiment, the LukB variant polypeptide is a CC8 LukB variant polypeptide comprising an amino acid substitution at any one or more amino acid residues corresponding to amino acid residues Glu45, Glu109, Thr121, and Arg154 of SEQ ID NO: 15. In any embodiment, the LukB variant polypeptide is a CC8 LukB variant polypeptide comprising an amino acid substitution at each amino acid residue corresponding to amino acid residues Glu45, Glu109, Thr121, and Arg154 of SEQ ID NO: 15. In any embodiment, the amino acid substitutions at each of these amino acid residues involves the introduction of a cysteine residue as described above. In one embodiment, an exemplary LukB variant polypeptide comprising cysteine amino acid substitutions at residues corresponding to Glu45, Glu109, Thr121, and Arg154 of SEQ ID NO: 39 comprises the amino acid sequence of SEQ ID NO: 21, or an amino acid sequence having at least 85%, at least 90%, at least 95%, at least 97%, or at least 99% sequence similarity to the amino acid sequence of SEQ ID NO: 21.

In any embodiment, the LukB variant polypeptide is a CC45 LukB variant polypeptide comprising an amino acid substitution at any one of amino acid residues corresponding to amino acid residues Glu45, Glu110, Thr122, and Arg155 of SEQ ID NO: 16. In any embodiment, the LukB variant polypeptide is a CC45 LukB variant polypeptide comprising an amino acid substitution at each amino acid residue corresponding to amino acid residues Glu45, Glu110, Thr122, and Arg155 of SEQ ID NO: 16. In any embodiment, the amino acid substitutions at each of these amino acid residues involves the introduction of a cysteine residue as described above. In another embodiment, the LukB variant polypeptide comprising cysteine amino acid substitutions at residues corresponding to Glu45, Glu110, Thr122, and Arg155 of SEQ ID NO: 39 comprises the amino acid sequence of SEQ ID NO: 22, or an amino acid sequence having at least 85%, at least 90%, at least 95%, at least 97%, or at least 99% sequence similarity to the amino acid sequence of SEQ ID NO: 22.

Other exemplary LukB variant polypeptides include any one of the LukB proteins of SEQ ID NOs: 40-51 comprising the described amino acid substitutions at residues corresponding to residues Glu45, Glu109, Thr121, and Arg154 of SEQ ID NO: 39.

In any embodiment, the LukB variant polypeptide as disclosed herein comprises an amino acid substitution at the amino acid residue corresponding to Val53 of SEQ ID NO: 39 in combination with an amino acid residue substitution at one or more amino acid residues corresponding to Glu45, Glu109, Thr121, and Arg154 of SEQ ID NO: 39. In any embodiment, the LukB variant polypeptide is a CC8 LukB variant polypeptide comprising an amino acid substitution at each amino acid residue corresponding to amino acid residues Val53, Glu45, Glu109, Thr121, and Arg154 of SEQ ID NO: 15. In any embodiment, the LukB variant polypeptide is a CC8 LukB variant polypeptide comprising an amino acid substitution at each amino acid residue corresponding to amino acid residues Val53Leu, Glu45Cys, Glu109Cys, Thr121Cys, and Arg154Cys of SEQ ID NO: 15. In any embodiment, an exemplary CC8 LukB variant polypeptide comprises the amino acid sequence of SEQ ID NO: 19, or an amino acid sequence having at least 85%, at least 90%, at least 95%, at least 97%, or at least 99% sequence similarity to the amino acid sequence of SEQ ID NO: 19.

In any embodiment, the LukB variant polypeptide is a CC45 LukB variant polypeptide comprising an amino acid substitution at each amino acid residue corresponding to amino acid residues Val53, Glu45, Glu110, Thr122, and Arg155 of SEQ ID NO: 16. In any embodiment, the LukB variant polypeptide is a CC45 LukB variant polypeptide comprising an amino acid substitution at each amino acid residue corresponding to amino acid residues Val53Leu, Glu45Cys, Glu110Cys, Thr123Cys, and Arg155Cys of SEQ ID NO: 16. An exemplary CC45 LukB variant polypeptide having amino acid substitutions at residues corresponding to each of the aforementioned positions has an amino acid sequence of SEQ ID NO: 20, or an amino acid sequence having at least 85%, at least 90%, at least 95%, at least 97%, or at least 99% sequence similarity to the amino acid sequence of SEQ TD NO: 20.

Other exemplary LukB variant polypeptides include any one of the LukB proteins of SEQ TD NOs: 40-51 comprising the described amino acid substitution at residues corresponding to Val53, Glu45, Glu109, Thr121, and Arg154 of SEQ ID NO: 39.

Table 2 below provides exemplary variant LukB amino acid sequences as disclosed herein.

TABLE 2
Exemplary LukB Polypeptide Amino Acid Sequences

| SEQ | | |
|---|---|---|
| ID NO | Name | Description |
| 15 | LukB CC8 WT | KINSEIKQVSEKNLDGDTKMYTRTATTSDSQK |
| | | NITQSLQFNFLTEPNYDKETVFIKAKGTIGSGLR |
| | | ILDPNGYWNSTLRWPGSYSVSIQNVDDNNNTN |
| | | VTDFAPKNQDESREVKYTYGYKTGGDFSINRG |
| | | GLTGNITKESNYSETISYQQPSYRTLLDQSTSHK |
| | | GVGWKVEAHLINNMGHDHTRQLTNDSDNRTK |
| | | SEIFSLTRNGNLWAKDNFTPKDKMPVTVSEGF |
| | | NPEFLAVMSHDKKDKGKSQFVVHYKRSMDEF |
| | | KIDWNRHGFWGYWSGENHVDKKEEKLSALYE |
| | | VDWKTHNVKFVKVLNDNEKK |
| 16 | LukB CC45 WT | EIKSKITTVSEKNLDGDTKMYTRTATTSDTEKK |
| | | ISQSLQFNFLTEPNYDKETVFIKAKGTIGSGLKI |
| | | LNPNGYWNSTLRWPGSYSVSIQNVDDNNNST |
| | | NVTDFAPKNQDESREVKYTYGYKTGGDFSINR |
| | | GGLTGNITKEKNYSETISYQQPSYRTLIDQPTTN |
| | | KGVAWKVEAHSINNMGHDHTRQLTNDSDDR |
| | | VKSEIFSLTRNGNLWAKDNFTPKNKMPVTVSE |
| | | GFNPEFLAVMSHDKNDKGKSRFIVHYKRSMD |
| | | DFKLDWNKHGFWGYWSGENHVDQKEEKLSA |
| | | LYEVDWKTHDVKLIKTFNDKEKK |
| 17 | LukB CC8 Val53Leu | KINSEIKQVSEKNLDGDTKMYTRTATTSDSQK |
| | | NITQSLQFNFLTEPNYDKETLFIKAKGTIGSGLR |
| | | ILDPNGYWNSTLRWPGSYSVSIQNVDDNNNTN |
| | | VTDFAPKNQDESREVKYTYGYKTGGDFSINRG |
| | | GLTGNITKESNYSETISYQQPSYRTLLDQSTSHK |
| | | GVGWKVEAHLINNMGHDHTRQLTNDSDNRTK |
| | | SEIFSLTRNGNLWAKDNFTPKDKMPVTVSEGF |
| | | NPEFLAVMSHDKKDKGKSQFVVHYKRSMDEF |
| | | KIDWNRHGFWGYWSGENHVDKKEEKLSALYE |
| | | VDWKTHNVKFVKVLNDNEKK |
| 18 | LukB CC45 Val53Leu | EIKSKITTVSEKNLDGDTKMYTRTATTSDTEKK |
| | | ISQSLQFNFLTEPNYDKETLFIKAKGTIGSGLKI |
| | | LNPNGYWNSTLRWPGSYSVSIQNVDDNNNST |
| | | NVTDFAPKNQDESREVKYTYGYKTGGDFSINR |
| | | GGLTGNITKEKNYSETISYQQPSYRTLIDQPTIN |
| | | KGVAWKVEAHSINNMGHDHTRQLTNDSDDR |
| | | VKSEIFSLTRNGNLWAKDNFTPKNKMPVTVSE |
| | | GFNPEFLAVMSHDKNDKGKSRFIVHYKRSMD |
| | | DFKLDWNKHGFWGYWSGENHVDQKEEKLSA |
| | | LYEVDWKTHDVKLIKTFNDKEKK |
| 19 | LukB CC8 Val53Leu, | KINSEIKQVSEKNLDGDTKMYTRTATTSDSQK |
| | Glu45Cys, Glu109Cys, | NITQSLQFNFLTCPNYDKETLFIKAKGTIGSGLR |
| | Thr121Cys, and Arg154Cys | ILDPNGYWNSTLRWPGSYSVSIQNVDDNNNTN |
| | | VTDFAPKNQDCSREVKYTYGYKCGGDFSINRG |
| | | GLTGNITKESNYSETISYQQPSYCTLLDQSTSH |
| | | KGVGWKVEAHLINNMGHDHTRQLTNDSDNRT |
| | | KSEIFSLTRNGNLWAKDNFTPKDKMPVTVSEG |
| | | FNPEFLAVMSHDKKDKGKSQFVVHYKRSMDE |
| | | FKIDWNRHGFWGYWSGENHVDKKEEKLSALY |
| | | EVDWKTHNVKFVKVLNDNEKK |
| 20 | LukB CC45 Val53Leu, | EIKSKITTVSEKNLDGDTKMYTRTATTSDTEKK |
| | Glu45Cys, Thr122Cys, | ISQSLQFNFLTCPNYDKETLFIKAKGTIGSGLKI |
| | Glu110Cys, Arg155Cys | LNPNGYWNSTLRWPGSYSVSIQNVDDNNNST |
| | | NVTDFAPKNQDCSREVKYTYGYKCGGDFSINR |
| | | GGLTGNITKEKNYSETISYQQPSYCTLIDQPTTN |
| | | KGVAWKVEAHSINNMGHDHTRQLTNDSDDR |
| | | VKSEIFSLTRNGNLWAKDNFTPKNKMPVTVSE |
| | | GFNPEFLAVMSHDKNDKGKSRFIVHYKRSMD |
| | | DFKLDWNKHGFWGYWSGENHVDQKEEKLSA |
| | | LYEVDWKTHDVKLIKTFNDKEKK |
| 21 | LukB CC8 Glu45Cys, | KINSEIKQVSEKNLDGDTKMYTRTATTSDSQK |
| | Glu109Cys, Thr121Cys, | NITQSLQFNFLTCPNYDKETVFIKAKGTIGSGLR |
| | and Arg154Cys | ILDPNGYWNSTLRWPGSYSVSIQNVDDNNNTN |
| | | VTDFAPKNQDCSREVKYTYGYKCGGDFSINRG |
| | | GLTGNITKESNYSETISYQQPSYCTLLDQSTSH |
| | | KGVGWKVEAHLINNMGHDHTRQLTNDSDNRT |
| | | KSEIFSLTRNGNLWAKDNFTPKDKMPVTVSEG |
| | | FNPEFLAVMSHDKKDKGKSQFVVHYKRSMDE |
| | | FKIDWNRHGFWGYWSGENHVDKKEEKLSALY |
| | | EVDWKTHNVKFVKVLNDNEKK |
| 22 | LukB CC45 Glu45Cys, | EIKSKITTVSEKNLDGDTKMYTRTATTSDTEKK |
| | Thr122Cys, Glu110Cys, | ISQSLQFNFLTCPNYDKETVFIKAKGTIGSGLKI |
| | Arg155Cys | LNPNGYWNSTLRWPGSYSVSIQNVDDNNNST |
| | | NVTDFAPKNQDCSREVKYTYGYKCGGDFSINR |
| | | GGLTGNITKEKNYSETISYQQPSYCTLIDQPTTN |
| | | KGVAWKVEAHSINNMGHDHTRQLTNDSDDR |
| | | VKSEIFSLTRNGNLWAKDNFTPKNKMPVTVSE |
| | | GFNPEFLAVMSHDKNDKGKSRFIVHYKRSMD |
| | | DFKLDWNKHGFWGYWSGENHVDQKEEKLSA |
| | | LYEVDWKTHDVKLIKTFNDKEKK |

In accordance with all aspects of the disclosure herein, the variant LukA and/or LukB variant polypeptides disclosed herein may further comprise one or more heterologous amino acid sequences. Suitable heterologous amino acid sequences include, without limitation, a tag sequences, immunogens, signal sequences, etc. Suitable tag sequences include, without limitation, a polyhistidine-tag, a polyarginine tag, FLAG tag, Step-tag II, ubiquitin tag, a NusA tag, a chitin binding domain, a calmodulin-binding peptide, cellulose-binding domain, Hat-tag, S-tag, SBP, maltose-binding protein, glutathione S-transferase (see Terpe K., “Overview of Tag Protein Fusions: From Molecular and Biochemical Fundamentals to Commercial Systems,” Appl. Microbiol. Biotechnol. 60:523-33 (2003), which is hereby incorporated by reference). Suitable immunogens include, without limitation, a T-cell epitope, a B-cell epitope. Suitable signal sequences include, without limitation, a PelB signal sequence, a Sec signal sequence, a Tat signal sequence, an AmyE signal sequence (see Freudl R., “Signal Peptides for Recombinant Protein Secretion in Bacterial Expression Systems,” Microbial Cell Factories 17:52 (2018), which is hereby incorporated by reference. In any embodiment, the LukA and/or LukB variant polypeptides as described herein comprise a PelB sequence (MKYLLPTAAAGLLLLAAQPAMA; SEQ ID NO: 23). In any embodiment, the LukA and/or LukB variant polypeptides thereof as described herein comprise His-tag (e.g., NSAHHHHHHGS; SEQ ID NO: 24). In any embodiment the LukA and/or LukB variant polypeptides as described herein comprise both the aforementioned PelB sequence and His-tag.

### Variant LukA and LukB Polynucleotides and Constructs

Another aspect of the present disclosure is directed to nucleic acid molecules encoding the LukA and LukB variant polypeptides as described herein. The nucleic acid molecules described herein include isolated polynucleotides, recombinant polynucleotide sequences, portions of expression vectors or portions of linear DNA or RNA sequences, including linear DNA or RNA sequences used for in vitro or in vivo transcription/translation, and vectors compatible with prokaryotic and eukaryotic cell expression and secretion of the LukA and LukB variant polypeptides thereof as described herein. In any embodiment, the LukA and LukB polynucleotides as described herein comprise DNA. In any embodiment, the LukA and LukB polynucleotides as described herein comprise RNA, in particular mRNA.

The polynucleotides of the disclosure may be produced by chemical synthesis such as solid phase polynucleotide synthesis on an automated polynucleotide synthesizer and assembled into complete single or double stranded molecules. Alternatively, the polynucleotides of the disclosure may be produced by other techniques such a PCR followed by routine cloning. Techniques for producing or obtaining polynucleotides of a given sequence are well known in the art.

In any embodiment, a polynucleotide of the present disclosure encodes a LukA variant polypeptide comprising a lysine to methionine substitution at the residue corresponding to the lysine at position 83 (Lys83Met) of SEQ ID NO: 25. In any embodiment, a polynucleotide of the present disclosure encodes the LukA variant polypeptide comprising a serine to alanine substitution at the residue corresponding to the serine at position 141 (Ser141Ala) of SEQ ID NO: 25. In any embodiment, a polynucleotide of the present disclosure encodes a LukA variant polypeptide comprising a valine to isoleucine substitution at the residue corresponding to the valine at position 113 (Val113Ile) of SEQ ID NO: 25. In any embodiment, a polynucleotide of the present disclosure encodes a LukA variant polypeptide comprising a valine to isoleucine substitution at the residue corresponding to the valine at position 193 (Val193Ile) of SEQ ID NO: 25. In any embodiment, a polynucleotide of the present disclosure encodes a LukA variant polypeptide comprising the amino acid substitutions of lysine to methionine, serine to alanine, and valine to isoleucine at residues corresponding to the aforementioned amino acid residues, i.e., Lys803Met, Ser141Ala, Val113Ile, and Val193Ile of SEQ ID NO: 25. In any embodiment, the polynucleotide of the present disclosure encodes a LukA variant polypeptide further comprising the amino acid substitution corresponding to Glu323Ala, i.e., the polynucleotide encodes a LukA variant comprising substitutions corresponding to the Lys83Met, Ser141Ala, Val113Ile, Val193Ile, and Glu323Ala substitutions of SEQ ID NO: 25.

In one embodiment, an exemplary nucleic acid molecule is a nucleic acid molecule encoding a CC8 LukA variant sequence, e.g., encoding a variant of SEQ ID NO: 1 comprising amino acid substitutions corresponding to Lys80Met, Ser138Ala, Val110Ile, Val190Ile, and Glu320Ala in SEQ ID NO: 1. An exemplary nucleic acid molecule encoding CC8 LukA is provided herein as SEQ ID NO: 52. Accordingly, in any embodiment, an exemplary nucleic acid molecule is a variant of SEQ ID NO: 52, wherein said variant comprises a nucleotide sequence having at least 85%, at least 90%, at least 95%, at least 97%, or at least 99% sequence similarity to the amino acid sequence of SEQ ID NO: 52.

In one embodiment, an exemplary nucleic acid molecule encoding a CC8 Luk8 variant is a nucleic acid molecule encoding the LukA variant sequence of SEQ ID NO: 3 (LukA CC8 Glu320Ala, Lys80Met, Ser138Ala, Val110Ile, Val190Ile) or an amino acid sequence having at least 85%, at least 90%, at least 95%, at least 97%, or at least 99% sequence similarity to the amino acid sequence of SEQ ID NO: 3. An exemplary nucleic acid molecule encoding this LukA CC8 variant comprises a nucleotide sequence having at least 85%, at least 90%, at least 95%, at least 97%, or at least 99% sequence similarity to SEQ ID NO: 54. In any embodiment, the nucleic acid molecule encoding this LukA CC8 variant comprises the nucleotide sequence of SEQ ID NO: 54.

In another embodiment, an exemplary nucleic acid molecule is a nucleic acid molecule encoding a CC45 LukA variant sequence, e.g., encoding a variant of SEQ ID NO: 2 comprising amino acid substitutions corresponding to Lys81Met, Ser139Ala, Val111Ile, Val191Ile, and Glu321Ala in SEQ ID NO: 2. An exemplary nucleic acid molecule encoding CC45 LukA is provided herein as SEQ ID NO: 53. Accordingly, in any embodiment, an exemplary nucleic acid molecule is a variant of SEQ ID NO: 53, wherein said variant comprises a nucleotide sequence having at least 85%, at least 90%, at least 95%, at least 97%, or at least 99% sequence similarity to the amino acid sequence of SEQ ID NO: 53.

In one embodiment, an exemplary nucleic acid molecule of the present disclosure is a nucleic acid molecule encoding the CC45 LukA variant sequence of SEQ ID NO: 4 (LukA CC45 Glu321Ala, Lys81Met, Ser139Ala, Val111Ile, Val191Ile) or an amino acid sequence having at least 85%, at least 90%, at least 95%, at least 97%, or at least 99% sequence similarity to the amino acid sequence of SEQ ID NO: 4. An exemplary nucleic acid molecule encoding this LukA CC45 variant comprises a nucleotide sequence having at least 85%, at least 90%, at least 95%, at least 97%, or at least 99% sequence similarity to SEQ ID NO: 55. In any embodiment, the nucleic acid molecule encoding this LukA CC8 variant comprises the nucleotide sequence of SEQ ID NO: 55.

In any embodiment, the polynucleotide of the present disclosure encodes a LukA variant polypeptide comprising an amino acid substitution at one or more amino acid residues corresponding to amino acid residues Tyr74, Asp140, Gly149, and Gly156 of SEQ ID NO: 25. In one embodiment, the polynucleotide encodes a LukA variant polypeptide comprising a tyrosine to cysteine substitution at the amino acid residue corresponding to Tyr74 (Tyr74Cys) of SEQ ID NO: 25, and comprises an asparagine to cysteine substitution at the amino acid residue corresponding to Asp140 (Asp140Cys) of SEQ ID NO: 25. In any embodiment, the polynucleotide of the present disclosure encodes a LukA variant polypeptide comprising a glycine to cysteine substitution at the amino acid residue corresponding to Gly149 (Gly149Cys) of SEQ ID NO: 25, and comprises a glycine to cysteine substitution at the amino acid residue corresponding to Gly156 (Gly156Cys) of SEQ ID NO: 25. In any embodiment, the polynucleotide of the present disclosure encodes a LukA variant polypeptide comprising amino acid substitutions at each amino acid residue corresponding to amino acid residues Tyr74, Asp140, Gly149, and Gly156 of SEQ ID NO: 25. In any embodiment, the amino acid substitution at each of these amino acid residues is a cysteine residue as described above.

In any embodiment, the polynucleotide of the present disclosure encodes a LukA variant polypeptide comprising amino acid substitution at one or more amino acid residues corresponding to Lys83, Ser141, Val113, Val193, and Glu323 in combination with an amino acid substitution at one or more amino acid residues corresponding to amino acid residues Tyr74, Asp140, Gly149, and Gly156 of SEQ ID NO: 25. In any embodiment, the polynucleotide encodes a LukA variant polypeptide comprising amino acid substitutions at amino acid residues corresponding to residues Lys83, Ser141, Val13, Val193, and Glu323 and residues Tyr74, Asp140, Gly149, and Gly156 of SEQ ID NO: 25.

In one embodiment, an exemplary nucleic acid molecule is a nucleic acid molecule encoding a CC8 LukA variant sequence, e.g., encoding a variant of SEQ ID NO: 1 comprising amino acid substitutions corresponding to each of Lys80Met, Ser138Ala, Val110Ile, Val190Ile, Glu320Ala, Tyr71Cys, Asp137Cys, Gly146Cys, and Gly153Cys of SEQ ID NO: 1. In one embodiment, an exemplary nucleic acid molecule is a nucleic acid molecule encoding the LukA variant sequence of SEQ ID NO: 5 (LukA CC8 Glu320Ala, Lys80Met, Ser138Ala, Val110Ile, Val190Ile, Tyr71Cys, Asp137Cys, Gly146Cys, Gly153Cys) or an amino acid sequence having at least 85%, at least 90%, at least 95%, at least 97%, or at least 99% sequence similarity to the amino acid sequence of SEQ ID NO: 5.

In another embodiment, an exemplary nucleic acid molecule is a nucleic acid molecule encoding a CC45 LukA variant sequence, e.g., encoding a variant of SEQ ID NO: 2 comprising amino acid substitutions corresponding to each of Lys81Met, Ser139Ala, Val111Ile, Val191Ile, Glu321Ala, Tyr72Cys, Asp138Cys, Gly147Cys, and Gly154Cys of SEQ ID NO: 2. In one embodiment, an exemplary nucleic acid molecule of the present disclosure is a nucleic acid molecule encoding the LukA variant sequence of SEQ ID NO: 6 (LukA CC45 Glu321Ala, Lys81Met, Ser139Ala, Val111Ile, Val191Ile, Tyr72Cys, Asp138Cys, Gly147Cys, Gly154Cys) or an amino acid sequence having at least 85%, at least 90%, at least 95%, at least 97%, or at least 99% sequence similarity to the amino acid sequence of SEQ ID NO: 6.

In any embodiment, the polynucleotide of the present disclosure encodes a LukA variant polypeptide comprising an amino acid substitution or deletion at the amino acid residue corresponding to amino acid residue Thr249 of SEQ ID NO: 25. In any embodiment, the polynucleotide encodes a LukA variant comprising a threonine to valine substitution at this residue corresponding to position 249 of SEQ ID NO: 25. In any embodiment, the polynucleotide of the present disclosure encodes a LukA variant polypeptide comprising the amino acid substitution at the position corresponding to Thr249 in combination with any one of or all of the amino acid substitutions at residues corresponding to Lys83, Ser141, Val13, Val193, Glu323, Tyr74, Asp140, Gly149, and Gly156 of SEQ ID NO: 25.

In one embodiment, an exemplary nucleic acid molecule is a nucleic acid molecule encoding a CC8 LukA variant sequence, e.g., encoding a variant of SEQ ID NO: 1 comprising amino acid substitutions corresponding to each of Lys80Met, Ser138Ala, Val110Ile, Val190Ile, Glu320Ala, and Thr246Val of SEQ ID NO: 1. In any embodiment, an exemplary nucleic acid molecule of the present disclosure is a nucleic acid molecule encoding the LukA variant sequence of SEQ ID NO: 7 (LukA CC8 Glu320Ala, Lys80Met, Ser138Ala, Val110Ile, Val190Ile, and Thr246Val) or an amino acid sequence having at least 85%, at least 90%, at least 95%, at least 97%, or at least 99% sequence similarity to the amino acid sequence of SEQ ID NO: 7. In any embodiment, an exemplary nucleic acid molecule of the present disclosure is a nucleic acid molecule encoding the CC8 LukA variant sequence of SEQ ID NO: 9 comprising the amino acid substitutions corresponding to Glu320Ala, Lys80Met, Ser138Ala, Val111Ile, Val190Ile, Thr246Val, Tyr71Cys, Asp137Cys, Gly146Cys, and Gly153Cys. Exemplary nucleic acid molecules of the present disclosure encompass nucleic acid molecules encoding an amino acid sequence having at least 85%, at least 90%, at least 95%, at least 97%, or at least 99% sequence similarity to the amino acid sequence of SEQ ID NO: 9. In any embodiment, an exemplary nucleic acid molecule encoding this LukA CC8 variant of SEQ ID NO: 9 comprises a nucleotide sequence having at least 85%, at least 90%, at least 95%, at least 97%, or at least 99% sequence similarity to SEQ ID NO: 56. In any embodiment, the nucleic acid molecule encoding this LukA CC8 variant comprises the nucleotide sequence of SEQ ID NO: 56.

In another embodiment, an exemplary nucleic acid molecule is a nucleic acid molecule encoding a CC45 LukA variant sequence, e.g., encoding a variant of SEQ ID NO: 2 comprising amino acid substitutions corresponding to each of Lys81Met, Ser139Ala, Val111Ile, Val191Ile, Glu321Ala, and Thr247Val of SEQ ID NO: 2. In any embodiment, an exemplary nucleic acid molecule of the present disclosure is a nucleic acid molecule encoding the LukA variant sequence of SEQ ID NO: 8 (LukA CC45 Glu321Ala, Lys81Met, Ser139Ala, Val111Ile, Val191Ile, Thr247Val) or an amino acid sequence having at least 85%, at least 90%, at least 95%, at least 97%, or at least 99% sequence similarity to the amino acid sequence of SEQ ID NO: 8. In any embodiment, an exemplary nucleic acid molecule of the present disclosure is a nucleic acid molecule encoding the LukA variant sequence of SEQ ID NO: 10 comprising the amino acid substitutions corresponding to Glu321Ala, Lys81Met, Ser139Ala, Val111Ile, Val191Ile, Thr247Val, Tyr72Cys, Asp138Cys, Gly147Cys, and Gly154Cys. Exemplary nucleic acid molecules of the present disclosure encompass nucleic acid molecules encoding an amino acid sequence having at least 85%, at least 90%, at least 95%, at least 97%, or at least 99% sequence similarity to the amino acid sequence of SEQ ID NO:10. In any embodiment, an exemplary nucleic acid molecule encoding this LukA CC45 variant of SEQ ID NO: 10 comprises a nucleotide sequence having at least 85%, at least 90%, at least 95%, at least 97%, or at least 99% sequence similarity to SEQ ID NO: 57. In any embodiment, the nucleic acid molecule encoding this LukA CC8 variant comprises the nucleotide sequence of SEQ ID NO: 57.

Another aspect of the disclosure is directed to polynucleotides encoding a LukB variant polypeptide as disclosed herein. In one embodiment, the polynucleotide encodes a LukB variant polypeptide comprising an amino acid substitution or deletion at the amino acid residue corresponding to amino acid residue Val53 of SEQ ID NO: 39. In any embodiment, the amino acid substitution at Val53 comprises a valine to leucine (Val53Leu) substitution. In one embodiment, an exemplary nucleic acid molecule is a nucleic acid molecule encoding a CC8 LukB variant sequence, e.g., encoding a variant of SEQ ID NO: 15 comprising an amino acid substitution at position 53 of SEQ ID NO: 15. An exemplary nucleic acid molecule encoding CC8 LukB is provided herein as SEQ ID NO: 58. Accordingly, in any embodiment, an exemplary nucleic acid molecule is a variant of SEQ ID NO: 58, wherein said variant comprises a nucleotide sequence having at least 85%, at least 90%, at least 95%, at least 97%, or at least 99% sequence similarity to the amino acid sequence of SEQ ID NO: 58.

In one embodiment, an exemplary nucleic acid molecule of the present disclosure encodes a LukB variant polypeptide of SEQ ID NO: 17 (LukB CC8 V53L) or an amino acid sequence having at least 85%, at least 90%, at least 95%, at least 97%, or at least 99% sequence similarity to the amino acid sequence of SEQ ID NO: 17. An exemplary nucleic acid molecule encoding this LukB CC8 V53L variant comprises a nucleotide sequence having at least 85%, at least 90%, at least 95%, at least 97%, or at least 99% sequence similarity to SEQ ID NO: 60. In any embodiment, the nucleic acid molecule encoding this LukA CC8 variant comprises the nucleotide sequence of SEQ ID NO: 60.

In another embodiment, an exemplary nucleic acid molecule is a nucleic acid molecule encoding a CC45 LukB variant sequence, e.g., encoding a variant of SEQ ID NO: 16 comprising an amino acid substitution at position 53 of SEQ ID NO: 16. An exemplary nucleic acid molecule encoding CC45 LukB is provided herein as SEQ ID NO: 59. Accordingly, in any embodiment, an exemplary nucleic acid molecule is a variant of SEQ ID NO: 59, wherein said variant comprises a nucleotide sequence having at least 85%, at least 90%, at least 95%, at least 97%, or at least 99% sequence similarity to the amino acid sequence of SEQ ID NO: 59.

In another embodiment, an exemplary polynucleotide of the present disclosure encodes a LukB variant polypeptide of SEQ ID NO: 18 (LukB CC45 V53L), or an amino acid sequence having at least 85%, at least 90%, at least 95%, at least 97%, or at least 99% sequence similarity to the amino acid sequence of SEQ ID NO: 18. An exemplary nucleic acid molecule encoding this LukB CC45 V53L variant comprises a nucleotide sequence having at least 85%, at least 90%, at least 95%, at least 97%, or at least 99% sequence similarity to SEQ ID NO: 61. In any embodiment, the nucleic acid molecule encoding this LukA CC45 variant comprises the nucleotide sequence of SEQ ID NO: 61.

In any embodiment, the polynucleotide of the present disclosure encodes a LukB variant polypeptide comprising an amino acid substitution at one or more amino acid residues corresponding to amino acid residues Glu45, Glu109, Thr121, and Arg154 of SEQ ID NO: 39. In one embodiment, the amino acid substitution at the one or more aforementioned residues introduces one or more cysteine residues capable of forming a disulfide bond to stabilize conformation of the LukAB heterodimer structure. In one embodiment, the polynucleotide encodes a LukB variant protein or polypeptide comprising a glutamic acid to cysteine substitution at the amino acid residue corresponding to Glu45 (Glu45Cys) of SEQ ID NO: 39, and threonine to cysteine substitution at the amino acid residue corresponding to Thr121 (Thr121Cys) of SEQ ID NO: 39. In another embodiment, the polynucleotide encodes a LukB variant protein or polypeptide comprising a glutamic acid to cysteine substitution at the amino acid residue corresponding to Glu109 (Glu109Cys) of SEQ ID NO: 39, and an arginine to cysteine substitution at the amino acid residue corresponding to Arg154 (Arg154Cys) of SEQ ID NO:39.

In any embodiment, the polynucleotide of the present disclosure encodes a LukB variant polypeptide comprising amino acid substitutions at each amino acid residue corresponding to amino acid residues Glu45, Glu109, Thr121, and Arg154 of SEQ ID NO: 39. In any embodiment, the amino acid substitutions at each of these amino acid residues involves the introduction of a cysteine residue as described above.

In one embodiment, an exemplary nucleic acid molecule is a nucleic acid molecule encoding a CC8 LukB variant sequence, e.g., encoding a variant of SEQ ID NO: 15 comprising amino acid substitution at each amino acid residue corresponding to amino acid residues Glu45, Glu109, Thr121, and Arg154 of SEQ ID NO: 15. In one embodiment, the polynucleotide encodes a LukB variant polypeptide comprising the amino acid sequence of SEQ ID NO: 21 (LukB CC8 Glu45Cys, Glu109Cys, Thr121Cys, and Arg154Cys) or an amino acid sequence having at least 85%, at least 90%, at least 95%, at least 97%, or at least 99% sequence similarity to the amino acid sequence of SEQ ID NO: 21. In another embodiment, an exemplary nucleic acid molecule is a nucleic acid molecule encoding a CC45 LukB variant sequence, e.g., encoding a variant of SEQ ID NO: 16 comprising an amino acid substitution at each amino acid residue corresponding to amino acid residues Glu45, Glu110, Thr122, and Arg155 of SEQ ID NO: 16. In any embodiment, the polynucleotide encodes a LukB variant polypeptide comprising the amino acid sequence of SEQ ID NO: 22 (LukB CC45 Glu45Cys, Thr122Cys, Glu110Cys, Arg155Cys) or an amino acid sequence having at least 85%, at least 90%, at least 95%, at least 97%, or at least 99% sequence similarity to the amino acid sequence of SEQ ID NO: 22.

In any embodiment, the polynucleotide of the present disclosure encodes a LukB variant polypeptide comprising an amino acid substitution at the amino acid residue corresponding to Val53 of SEQ ID NO: 39 in combination with an amino acid residue substitution at one or more amino acid residues corresponding to Glu45, Glu109, Thr121, and Arg154 of SEQ ID NO: 39. In one embodiment, an exemplary nucleic acid molecule is a nucleic acid molecule encoding a CC8 LukB variant sequence, e.g., encoding a variant of SEQ ID NO: 15 comprising an amino acid substitution at each amino acid residue corresponding to amino acid residues Val53, Glu45, Glu109, Thr121, and Arg154 of SEQ ID NO: 15. In any embodiment, the polynucleotide encodes the LukB variant polypeptide having the amino acid sequence of SEQ ID NO: 19 (LukB CC8 Val53Leu, Glu45Cys, Glu109Cys, Thr121Cys, and Arg154Cys) or an amino acid sequence having at least 85%, at least 90%, at least 95%, at least 97%, or at least 99% sequence similarity to the amino acid sequence of SEQ ID NO: 19. In another embodiment, an exemplary nucleic acid molecule is a nucleic acid molecule encoding a CC45 LukB variant sequence, e.g., encoding a variant of SEQ ID NO: 16 comprising an amino acid substitution at each amino acid residue corresponding to amino acid residues Val53, Glu45, Glu110, Thr122, and Arg155 of SEQ ID NO: 16. In any embodiment, the polynucleotide encodes a LukB variant polypeptide having the amino acid sequence of SEQ ID NO: 20 (LukB CC45 Val53Leu, Glu45Cys, Thr122Cys, Glu110Cys, Arg155Cys) or an amino acid sequence having at least 85%, at least 90%, at least 95%, at least 97%, or at least 99% sequence similarity to the amino acid sequence of SEQ ID NO: 20.

In another embodiment, an exemplary nucleic acid molecule of the present disclosure is a nucleic acid molecule encoding a LukA sequence and a LukB sequence as disclosed herein. In one embodiment, an exemplary nucleic acid molecule is a polynucleotide encoding a CC45 LukA sequence (variant or non-variant) and a CC45 LukB sequence (variant or non-variant). For example, a polynucleotide encoding a CC45 LukA variant sequence as disclosed herein and a CC45 LukB non-variant sequence, or a polynucleotide encoding a CC45 LukA non-variant sequence and a CC45 LukB variant sequence as disclosed herein.

In another embodiment, an exemplary nucleic acid molecule is a polynucleotide encoding a CC8 LukA sequence (variant or non-variant) and a CC8 LukB sequence (variant or non-variant). For example, a polynucleotide encoding a CC8 LukA variant sequence as disclosed herein and a CC8 LukB non-variant sequence, or a polynucleotide encoding a CC8 LukA non-variant sequence and a CC8 LukB variant sequence as disclosed herein.

In another embodiment, an exemplary nucleic acid molecule is a polynucleotide encoding a CC45 LukA sequence (variant or non-variant) and a CC8 LukB sequence (variant or non-variant). For example, a polynucleotide encoding a CC45 LukA variant sequence as disclosed herein and a CC8 LukB non-variant sequence, or a polynucleotide encoding a CC45 LukA non-variant sequence and a CC8 LukB variant sequence as disclosed herein.

In another embodiment, an exemplary nucleic acid molecule is one that encodes a CC8 LukA sequence (variant or non-variant) and CC45 LukB sequence (variant or non-variant). For example, a polynucleotide encoding a CC8 LukA variant sequence as disclosed herein and a CC45 LukB non-variant sequence, or a polynucleotide encoding a CC8 LukA non-variant sequence and a CC45 LukB variant sequence as disclosed herein.

In another embodiment, an exemplary nucleic acid molecule of the present disclosure is a polynucleotide encoding a LukA variant sequence and a LukB wild-type sequence. For example, a polynucleotide encoding a LukA variant sequence selected from any one of SEQ ID NOs: 3, 4, 5, 6, 7, 8, 9, 10, 11, 12, 13, or 14, in combination with a LukB wild-type (i.e., non-variant) sequence of SEQ ID NO: 15 or SEQ ID NO: 16.

In any embodiment, an exemplary nucleic acid molecule of the present disclosure is a polynucleotide encoding a LukA wild-type sequence and a LukB variant sequence. For example, a polynucleotide encoding a LukA wild-type sequence of SEQ ID NO: 1 or SEQ ID NO: 2 in combination with a LuB variant sequence selected from any one of SEQ ID NOs: 17, 18, 19, 20, 21, or 22.

In any embodiment, an exemplary nucleic acid molecule of the present disclosure is a polynucleotide encoding a LukA variant sequence and a LukB variant sequence. For example, a polynucleotide encoding a LukA variant sequence selected from any one of SEQ ID NOs: 3, 4, 5, 6, 7, 8, 9, 10, 11, 12, 13, or 14, in combination with a LukB variant sequence selected from any one of SEQ ID NOs: 17, 18, 19, 20, 21, or 22.

In any embodiment, an exemplary nucleic acid molecule of the present disclosure encodes a LukA variant sequence of SEQ ID NO: 3 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 4 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 5 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 6 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 7 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 8 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 9 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 10 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 11 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 12 and a LukB non-variant sequence of SEQ ID NOs: 15 or 16, a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 13 and a LukB variant sequence of SEQ ID NO: 17 or 18, a LukB variant sequence of SEQ ID NO: 19 or 20, or a LukB variant sequence of SEQ ID NO: 21 or 22. In any embodiment, an exemplary nucleic acid molecule of the present disclosure encodes a LukA variant sequence of SEQ ID NO: 14 and a LukB variant sequence of SEQ ID NO: 17 or 18, a LukB variant sequence of SEQ ID NO: 19 or 20, or a LukB variant sequence of SEQ ID NO: 21 or 22.

In any embodiment, an exemplary nucleic acid molecule of the present disclosure encodes a LukB variant sequence of SEQ ID NO: 17 and a LukA non-variant sequence of SEQ ID NOs: 1 or 2, a LukA variant sequence of SEQ ID NO: 3 or 4, a LukA variant sequence of SEQ ID NO: 5 or 6, a LukA variant sequence of SEQ ID NO: 7 or 8, a LukA variant sequence of SEQ ID NO: 9 or 10, a LukA variant sequence of SEQ ID NO: 11 or 12, or a LukA variant sequence of SEQ ID NO: 13 or 14. In any embodiment, an exemplary nucleic acid molecule of the present disclosure encodes a LukB variant sequence of SEQ ID NO: 18 and a LukA non-variant sequence of SEQ ID NOs: 1 or 2, a LukA variant sequence of SEQ ID NO: 3 or 4, a LukA variant sequence of SEQ ID NO: 5 or 6, a LukA variant sequence of SEQ ID NO: 7 or 8, a LukA variant sequence of SEQ ID NO: 9 or 10, a LukA variant sequence of SEQ ID NO: 11 or 12, or a LukA variant sequence of SEQ ID NO: 13 or 14. In any embodiment, an exemplary nucleic acid molecule of the present disclosure encodes a LukB variant sequence of SEQ ID NO: 19 and a LukA non-variant sequence of SEQ ID NOs: 1 or 2, a LukA variant sequence of SEQ ID NO: 3 or 4, a LukA variant sequence of SEQ ID NO: 5 or 6, a LukA variant sequence of SEQ ID NO: 7 or 8, a LukA variant sequence of SEQ ID NO: 9 or 10, a LukA variant sequence of SEQ ID NO: 11 or 12, or a LukA variant sequence of SEQ ID NO: 13 or 14. In any embodiment, an exemplary nucleic acid molecule of the present disclosure encodes a LukB variant sequence of SEQ ID NO: 20 and a LukA non-variant sequence of SEQ ID NOs: 1 or 2, a LukA variant sequence of SEQ ID NO: 3 or 4, a LukA variant sequence of SEQ ID NO: 5 or 6, a LukA variant sequence of SEQ ID NO: 7 or 8, a LukA variant sequence of SEQ ID NO: 9 or 10, a LukA variant sequence of SEQ ID NO: 11 or 12, or a LukA variant sequence of SEQ ID NO: 13 or 14. In any embodiment, an exemplary nucleic acid molecule of the present disclosure encodes a LukB variant sequence of SEQ ID NO: 21 and a LukA non-variant sequence of SEQ ID NOs: 1 or 2, a LukA variant sequence of SEQ ID NO: 3 or 4, a LukA variant sequence of SEQ ID NO: 5 or 6, a LukA variant sequence of SEQ ID NO: 7 or 8, a LukA variant sequence of SEQ TD NO: 9 or 10, a LukA variant sequence of SEQ TD NO: 11 or 12, or a LukA variant sequence of SEQ ID NO: 13 or 14. In any embodiment, an exemplary nucleic acid molecule of the present disclosure encodes a LukB variant sequence of SEQ ID NO: 22 and a LukA non-variant sequence of SEQ ID NOs: 1 or 2, a LukA variant sequence of SEQ ID NO: 3 or 4, a LukA variant sequence of SEQ ID NO: 5 or 6, a LukA variant sequence of SEQ ID NO: 7 or 8, a LukA variant sequence of SEQ ID NO: 9 or 10, a LukA variant sequence of SEQ ID NO: 11 or 12, or a LukA variant sequence of SEQ ID NO: 13 or 14.

In any embodiment, an exemplary nucleic acid molecule of the present disclosure encodes a CC45 LukA variant sequence of SEQ ID NO: 4 and a CC45 LukB sequence of SEQ ID NO: 16. An exemplary nucleic acid molecule encoding this LukAB heterodimer (RARPR-15) comprises a nucleotide sequence having at least 85%, at least 90%, at least 95%, at least 97%, or at least 99% sequence similarity to the nucleotide sequence of SEQ ID NO: 55 (CC45 LukA variant) operatively coupled to a nucleotide sequence having at least 85%, at least 90%, at least 95%, at least 97%, or at least 99% sequence similarity to the nucleotide sequence of SEQ ID NO: 59 (CC45 LukB). An exemplary nucleic acid molecule encoding this LukAB heterodimer comprises the nucleotide sequence of SEQ ID NO: 55 operatively coupled to the nucleotide sequence of SEQ ID NO: 59.

In any embodiment, an exemplary nucleic acid molecule of the present disclosure encodes a CC45 LukA variant sequence of SEQ ID NO: 4 and a CC45 LukB variant sequence of SEQ ID NO: 18. An exemplary nucleic acid molecule encoding this LukAB heterodimer (RARPR-30) comprises a nucleotide sequence having at least 85%, at least 90%, at least 95%, at least 97%, or at least 99% sequence similarity to the nucleotide sequence of SEQ ID NO: 55 (CC45 LukA variant) operatively coupled to a nucleotide sequence having at least 85%, at least 90%, at least 95%, at least 97%, or at least 99% sequence similarity to the nucleotide sequence of SEQ ID NO: 61 (CC45 LukB variant). An exemplary nucleic acid molecule encoding this LukAB heterodimer comprises the nucleotide sequence of SEQ ID NO: 55 operatively coupled to the nucleotide sequence of SEQ ID NO: 61.

In any embodiment, an exemplary nucleic acid molecule of the present disclosure encodes a CC8 LukA variant sequence of SEQ ID NO: 3 and a CC8 LukB sequence of SEQ ID NO: 15. An exemplary nucleic acid molecule encoding this LukAB heterodimer (RARPR-32) comprises a nucleotide sequence having at least 85%, at least 90%, at least 95%, at least 97%, or at least 99% sequence similarity to the nucleotide sequence of SEQ ID NO: 54 (CC8 LukA variant) operatively coupled to a nucleotide sequence having at least 85%, at least 90%, at least 95%, at least 97%, or at least 99% sequence similarity to the nucleotide sequence of SEQ ID NO: 58 (CC8 LukB). An exemplary nucleic acid molecule encoding this LukAB heterodimer comprises the nucleotide sequence of SEQ ID NO: 54 operatively coupled to the nucleotide sequence of SEQ ID NO: 58.

In any embodiment, an exemplary nucleic acid molecule of the present disclosure encodes a CC8 LukA variant sequence of SEQ ID NO: 3 and a CC45 LukB variant sequence of SEQ ID NO: 18. An exemplary nucleic acid molecule encoding this LukAB heterodimer (RARPR-33) comprises a nucleotide sequence having at least 85%, at least 90%, at least 95%, at least 97%, or at least 99% sequence similarity to the nucleotide sequence of SEQ ID NO: 54 (CC8 LukA variant) operatively coupled to a nucleotide sequence having at least 85%, at least 90%, at least 95%, at least 97%, or at least 99% sequence similarity to the nucleotide sequence of SEQ ID NO: 61 (CC45 LukB variant). An exemplary nucleic acid molecule encoding this LukAB heterodimer comprises the nucleotide sequence of SEQ ID NO: 54 operatively coupled to the nucleotide sequence of SEQ ID NO: 61.

In any embodiment, an exemplary nucleic acid molecule of the present disclosure encodes a CC8 LukA variant sequence of SEQ ID NO: 3 and a CC8 LukB variant sequence of SEQ ID NO: 17. An exemplary nucleic acid molecule encoding this LukAB heterodimer (RARPR-34) comprises a nucleotide sequence having at least 85%, at least 90%, at least 95%, at least 97%, or at least 99% sequence similarity to the nucleotide sequence of SEQ ID NO: 54 (CC8 LukA variant) operatively coupled to a nucleotide sequence having at least 85%, at least 90%, at least 95%, at least 97%, or at least 99% sequence similarity to the nucleotide sequence of SEQ ID NO: 60 (CC8 LukB variant). An exemplary nucleic acid molecule encoding this LukAB heterodimer comprises the nucleotide sequence of SEQ ID NO: 54 operatively coupled to the nucleotide sequence of SEQ ID NO: 60.

Exemplary nucleic acid molecule sequences of the present disclosure are provided in Table 3 below.

TABLE 3
Exemplary LukA and LukB Polynucleotide Sequences

| Construct | SEQ | |
|---|---|---|
| Name | ID NO: | DNA Sequence |
| CC8 LukA^{wt} | 52 | GCT CAC AAA GAT TCT CAG GAT CAA AAT AAG AAG |
| | | GAG CAC GTC GAC AAG TCT CAG CAG AAA GAC AAG |
| | | CGT AAT GTT ACA AAC AAG GAC AAA AAC AGC ACT |
| | | GCT CCA GAC GAC ATT GGA AAA AAC GGT AAG ATT |
| | | ACT AAA CGC ACC GAA ACG GTA TAT GAC GAA AAA |
| | | ACG AAC ATT TTG CAA AAC TTG CAG TTC GAT TTC ATT |
| | | GAC GAC CCC ACT TAT GAC AAG AAT GTC CTT CTG GTG |
| | | AAG AAG CAG GGC AGC ATT CAC TCA AAC TTG AAA |
| | | TTT GAG TCT CAC AAG GAG GAG AAG AAC TCC AAT |
| | | TGG CTG AAA TAC CCA TCA GAG TAC CAC GTT GAT TTT |
| | | CAA GTG AAA CGT AAC CGC AAA ACG GAA ATT TTG |
| | | GAC CAA TTG CCG AAA AAC AAG ATC TCC ACC GCG |
| | | AAA GTA GAC TCA ACA TTC AGT TAC TCT TCC GGC |
| | | GGA AAG TTC GAC AGC ACT AAG GGG ATC GGG CGC |
| | | ACT TCT TCC AAT TCG TAC TCG AAA ACG ATT TCT TAC |
| | | AAT CAG CAG AAT TAT GAC ACT ATC GCA TCT GGT |
| | | AAA AAT AAT AAC TGG CAC GTG CAT TGG TCG GTG |
| | | ATT GCT AAT GAT TTA AAG TAT GGA GGT GAG GTA |
| | | AAA AAT CGT AAT GAC GAG CTG CTG TTT TAC CGT |
| | | AAC ACT CGC ATC GCA ACC GTT GAA AAC CCG GAA |
| | | TTG TCC TTT GCC TCG AAA TAC CGC TAC CCT GCA TTA |
| | | GTT CGT TCA GGC TTT AAT CCC GAG TTT TTG ACT TAT |
| | | CTT TCC AAT GAA AAA TCG AAC GAG AAG ACT CAG |
| | | TTC GAG GTT ACG TAC ACC CGC AAT CAG GAC ATT TTG |
| | | AAG AAC CGT CCG GGA ATT CAC TAT GCG CCT CCC |
| | | ATC TTA GAG AAG AAT AAG GAT GGA CAA CGT TTG |
| | | ATC GTT ACA TAT GAA GTT GAC TGG AAA AAT AAG |
| | | ACC GTA AAG GTT GTG GAT AAG TAT TCG GAT GAT |
| | | AAT AAG CCC TAT AAA GAA GGG |
| CC45 LukA^{wt} | 53 | GCG AAC AAA GAT TCT CAG GAC CAG ACC AAA AAG |
| | | GAG CAC GTA GAC AAG GCC CAG CAA AAA GAG AAG |
| | | CGT AAT GTG AAC GAC AAA GAT AAG AAT ACT CCG |
| | | GGG CCA GAT GAT ATC GGC AAG AAC GGT AAA GTC |
| | | ACG AAG CGT ACA GTG TCT GAG TAT GAC AAA GAA |
| | | ACA AAC ATC CTG CAG AAC TTA CAA TTC GAC TTT ATT |
| | | GAT GAT CCA ACT TAC GAT AAG AAT GTG TTG CTG GTT |
| | | AAG AAA CAA GGT TCA ATC CAT TCT AAC TTG AAG |
| | | TTC GAG TCA CAC CGT AAC GAA ACG AAC GCG TCG |
| | | TGG TTG AAA TAT CCG TCA GAG TAT CAT GTT GAT TTT |
| | | CAA GTA CAA CGT AAT CCC AAA ACG GAA ATT TTG |
| | | GAC CAA TTA CCT AAA AAT AAG ATT AGC ACC GCC |
| | | AAG GTT GAC TCA ACT TTC TCC TAC TCA TTA GGA GGA |
| | | AAG TTC GAT TCG ACA AAA GGG ATC GGG CGT ACA |
| | | TCT TCG AAT AGC TAC AGT AAG AGC ATT AGC TAT |
| | | AAC CAG CAG AAC TAT GAT ACG ATT GCT TCA GGG |
| | | AAA AAT AAC AAC CGT CAC GTA CAT TGG TCA GTG |
| | | GTT GCG AAC GAT CTT AAA TAT GGA AAC GAG ATT |
| | | AAG AAT CGT AAC GAC GAA TTT TTG TTT TAC CGC AAT |
| | | ACA CGC CTT AGT ACC GTG GAA AAT CCC GAG CTG |
| | | TCC TTC GCG TCG AAG TAT CGC TAT CCG GCC CTT GTG |
| | | CGT TCG GGT TTC AAT CCC GAG TTC TTA ACA TAT ATT |
| | | TCC AAT GAG AAA ACT AAC GAC AAG ACT CGC TTC |
| | | GAA GTC ACC TAC ACT CGC AAC CAG GAC ATT CTG |
| | | AAA AAC AAG CCT GGA ATT CAT TAC GGG CAA CCA |
| | | ATT TTA GAG CAG AAT AAG GAT GGA CAG CGC TTT |
| | | ATT GTG GTA TAT GAG GTG GAC TGG AAG AAT AAG |
| | | ACA GTA AAA GTT GTG GAA AAG TAC TCT GAC CAG |
| | | AAT AAG CCC TAT AAA GAA GGA |
| CC8 | 62 | GCT CAC AAA GAT TCT CAG GAT CAA AAT AAG AAG |
| LukAΔ10C | | GAG CAC GTC GAC AAG TCT CAG CAG AAA GAC AAG |
| | | CGT AAT GTT ACA AAC AAG GAC AAA AAC AGC ACT |
| | | GCT CCA GAC GAC ATT GGA AAA AAC GGT AAG ATT |
| | | ACT AAA CGC ACC GAA ACG GTA TAT GAC GAA AAA |
| | | ACG AAC ATT TTG CAA AAC TTG CAG TTC GAT TTC ATT |
| | | GAC GAC CCC ACT TAT GAC AAG AAT GTC CTT CTG GTG |
| | | AAG AAG CAG GGC AGC ATT CAC TCA AAC TTG AAA |
| | | TTT GAG TCT CAC AAG GAG GAG AAG AAC TCC AAT |
| | | TGG CTG AAA TAC CCA TCA GAG TAC CAC GTT GAT TTT |
| | | CAA GTG AAA CGT AAC CGC AAA ACG GAA ATT TTG |
| | | GAC CAA TTG CCG AAA AAC AAG ATC TCC ACC GCG |
| | | AAA GTA GAC TCA ACA TTC AGT TAC TCT TCC GGC |
| | | GGA AAG TTC GAC AGC ACT AAG GGG ATC GGG CGC |
| | | ACT TCT TCC AAT TCG TAC TCG AAA ACG ATT TCT TAC |
| | | AAT CAG CAG AAT TAT GAC ACT ATC GCA TCT GGT |
| | | AAA AAT AAT AAC TGG CAC GTG CAT TGG TCG GTG |
| | | ATT GCT AAT GAT TTA AAG TAT GGA GGT GAG GTA |
| | | AAA AAT CGT AAT GAC GAG CTG CTG TTT TAC CGT |
| | | AAC ACT CGC ATC GCA ACC GTT GAA AAC CCG GAA |
| | | TTG TCC TTT GCC TCG AAA TAC CGC TAC CCT GCA TTA |
| | | GTT CGT TCA GGC TTT AAT CCC GAG TTT TTG ACT TAT |
| | | CTT TCC AAT GAA AAA TCG AAC GAG AAG ACT CAG |
| | | TTC GAG GTT ACG TAC ACC CGC AAT CAG GAC ATT TTG |
| | | AAG AAC CGT CCG GGA ATT CAC TAT GCG CCT CCC |
| | | ATC TTA GAG AAG AAT AAG GAT GGA CAA CGT TTG |
| | | ATC GTT ACA TAT GAA GTT GAC TGG AAA AAT AAG |
| | | ACC GTA AAG GTT GTG GAT AAG TAT |
| CC45 | 63 | GCAAATAAAGACTCTCAAGATCAGACTAAAAAGGAACAT |
| LukAΔ10C | | GTTGATAAGGCGCAACAAAAAGAAAAGCGTAATGTCAAT |
| | | GATAAGGACAAGAATACTCCGGGACCCGACGACATTGGC |
| | | AAGAACGGAAAGGTGACAAAGCGTACCGTTAGTGAGTAC |
| | | GACAAGGAAACAAATATCCTGCAGAACTTACAGTTCGAT |
| | | TTTATTGACGATCCTACCTATGACAAGAATGTCCTGTTGG |
| | | TGAAGAAACAGGGCAGCATTCATTCCAATTTAAAATTTG |
| | | AAAGCCATCGTAACGAAACAAATGCATCTTGGCTTAAAT |
| | | ACCCTTCTGAGTACCACGTAGATTTTCAGGTACAACGCAA |
| | | CCCAAAAACCGAAATTCTGGATCAACTGCCCAAGAATAA |
| | | AATTTCTACGGCTAAAGTTGACAGTACATTTAGCTACAGT |
| | | TTAGGGGGAAAGTTTGATAGTACAAAAGGAATTGGTCGT |
| | | ACTTCCAGTAACTCCTATTCGAAATCTATTTCCTATAATC |
| | | AACAGAATTACGACACCATCGCATCCGGTAAAAACAATA |
| | | ATCGCCACGTACATTGGAGTGTTGTCGCGAATGACTTAAA |
| | | GTACGGTAACGAAATCAAGAACCGCAACGACGAATTCTT |
| | | ATTCTATCGTAACACGCGTTTAAGCACCGTCGAGAACCCC |
| | | GAGTTATCCTTTGCTAGCAAATATCGCTATCCTGCGTTAG |
| | | TACGCTCAGGGTTCAATCCTGAGTTCTTAACCTACATCTC |
| | | CAACGAGAAAACTAATGATAAGACACGCTTCGAGGTGAC |
| | | CTACACCCGTAATCAGGATATCCTTAAAAATAAACCGGG |
| | | TATTCATTACGGGCAACCCATTTTAGAACAGAATAAGGA |
| | | CGGCCAACGTTTTATCGTGGTCTATGAGGTTGATTGGAAG |
| | | AACAAGACAGTGAAAGTGGTTGAAAAGTAT |
| CC8 LukA | 54 | CATAAAGATTCGCAGGATCAAAATAAGAAGGAGCATGTT |
| W95 | | GACAAGAGCCAGCAGAAAGACAAGCGCAATGTTACAAA |
| | | CAAAGATAAGAACTCTACAGCGCCCGATGACATTGGTAA |
| | | GAACGGCAAGATAACTAAGCGGACGGAAACCGTGTATGA |
| | | CGAGAAAACTAACATTCTGCAAAATTTGCAATTTGACTTT |
| | | ATCGACGATCCAACCTATGACAAGAATGTCTTGCTTGTCA |
| | | AAATGCAAGGTTCGATTCATTCAAACCTTAAATTTGAATC |
| | | CCACAAAGAGGAGAAAAACTCTAATTGGTTAAAGTATCC |
| | | TTCAGAATATCACATAGATTTCCAGGTAAAGAGAAACCG |
| | | TAAAACGGAGATACTGGATCAACTGCCTAAAAACAAGAT |
| | | CTCGACAGCTAAGGTGGACGCTACGTTCTCGTACTCGTCT |
| | | GGTGGGAAGTTCGACTCGACCAAAGGCATTGGGCGTACA |
| | | TCATCAAATAGCTATTCAAAGACTATTAGCTATAATCAGC |
| | | AGAACTATGATACGATAGCTTCGGGTAAGAATAACAACT |
| | | GGCACGTTCATTGGTCGATCATTGCAAATGACTTGAAGTA |
| | | TGGCGGAGAGGTAAAGAATCGCAACGATGAGCTGTTATT |
| | | CTATCGCAATACGAGAATTGCGACTGTAGAGAACCCGGA |
| | | ATTGTCTTTTGCCTCCAAATATCGGTACCCGGCATTGGTA |
| | | CGCTCTGGTTTCAATCCTGAGTTTTTAACTTACCTTTCCAA |
| | | CGAAAAGAGTAATGAGAAGACCCAATTTGAGGTTACCTA |
| | | CACCCGTAACCAGGATATTTTGAAGAATCGGCCGGGCAT |
| | | CCATTATGCCCCACCAATCCTGGAGAAAAATAAAGACGG |
| | | TCAGCGGCTTATTGTGACTTACGAGGTCGATTGGAAAAAT |
| | | AAGACGGTCAAGGTAGTGGACAAATATTCTGATGACAAT |
| | | AAACCGTACAAAGCTGGC |
| CC45 LukA | 55 | GCTAATAAGGACTCCCAGGACCAGACAAAGAAGGAACA |
| W95 | | CGTCGACAAAGCCCAGCAAAAAGAAAAACGCAACGTAA |
| | | ACGATAAGGACAAGAACACCCCAGGACCCGATGATATTG |
| | | GGAAGAACGGTAAAGTCACAAAACGCACAGTGAGCGAG |
| | | TACGATAAAGAAACAAATATCCTGCAAAATCTGCAATTT |
| | | GACTTCATCGATGACCCTACCTATGATAAGAATGTGTTGT |
| | | TGGTTAAGATGCAGGGAAGTATTCATTCCAACTTGAAATT |
| | | CGAGAGCCACCGTAACGAAACGAATGCGAGTTGGTTAAA |
| | | GTACCCTTCAGAATACCACATTGATTTTCAGGTGCAGCGT |
| | | AACCCGAAAACCGAAATCTTAGACCAGCTGCCTAAAAAC |
| | | AAGATTTCTACGGCCAAGGTGGACGCAACTTTCAGTTATA |
| | | GTCTTGGAGGAAAGTTCGACAGTACCAAAGGTATCGGCC |
| | | GCACATCCTCAAACAGCTATTCGAAATCCATTTCTTACAA |
| | | CCAGCAAAATTATGACACGATCGCCTCAGGTAAGAACAA |
| | | CAATCGTCATGTGCATTGGAGCATCGTGGCTAACGATTTG |
| | | AAATATGGTAACGAAATCAAAAATCGCAATGACGAGTTC |
| | | TTGTTTTACCGCAATACTCGCCTTTCTACGGTAGAGAATC |
| | | CTGAGCTTAGCTTTGCGAGCAAGTATCGTTACCCTGCTCT |
| | | TGTACGTTCGGGTTTCAACCCAGAGTTCCTTACTTATATC |
| | | TCCAATGAGAAGACGAACGATAAAACCCGTTTTGAAGTT |
| | | ACATACACGCGTAATCAGGACATCTTAAAGAATAAACCG |
| | | GGGATTCATTATGGGCAGCCGATCTTAGAGCAAAATAAG |
| | | GATGGACAGCGTTTCATTGTAGTGTATGAGGTTGACTGGA |
| | | AGAACAAGACGGTAAAAGTAGTTGAAAAGTATTCCGACC |
| | | AAAACAAGCCTTATAAGGCGGGT |
| CC8 LukA | 56 | CACAAAGACAGCCAGGATCAAAACAAGAAAGAGCACGT |
| W97 W92 | | GGACAAGAGCCAGCAAAAGGATAAACGTAACGTTACCA |
| | | ACAAGGACAAAAACAGCACCGCGCCGGACGATATCGGC |
| | | AAGAACGGCAAAATTACCAAGCGTACCGAGACCGTGTAC |
| | | GATGAAAAAACCAACATCCTGCAGAACCTGCAATTCGAC |
| | | TTTATTGACGATCCGACCTGCGATAAAAACGTGCTGCTGG |
| | | TTAAGATGCAGGGCAGCATCCACAGCAACCTGAAATTCG |
| | | AAAGCCACAAAGAGGAAAAGAACAGCAACTGGCTGAAG |
| | | TACCCGAGCGAGTATCACATTGACTTTCAGGTGAAACGT |
| | | AACCGTAAGACCGAAATCCTGGATCAACTGCCGAAGAAC |
| | | AAAATTAGCACCGCGAAGGTTTGCGCGACCTTCAGCTAC |
| | | AGCAGCGGTTGCAAATTTGACAGCACCAAGTGCATCGGC |
| | | CGTACCAGCAGCAACAGCTATAGCAAAACCATCAGCTAC |
| | | AACCAGCAAAACTATGATACCATTGCGAGCGGCAAGAAC |
| | | AACAACTGGCACGTGCACTGGAGCATCATTGCGAACGAC |
| | | CTGAAATACGGTGGCGAGGTTAAGAACCGTAACGATGAA |
| | | CTGCTGTTCTATCGTAACACCCGTATCGCGACCGTGGAGA |
| | | ACCCGGAACTGAGCTTTGCGAGCAAATACCGTTATCCGG |
| | | CGCTGGTGCGTAGCGGTTTCAACCCGGAGTTTCTGGTTTA |
| | | CCTGAGCAACGAGAAAAGCAACGAAAAGACCCAGTTCG |
| | | AAGTTACCTACACCCGTAACCAAGACATCCTGAAGAACC |
| | | GTCCGGGTATCCACTATGCTCCGCCGATTCTGGAGAAGA |
| | | ACAAAGATGGCCAACGTCTGATTGTGACCTATGAAGTTG |
| | | ACTGGAAGAACAAAACCGTTAAAGTGGTTGATAAGTACA |
| | | GCGACGATAACAAACCGTATAAGGCGGGT |
| CC45 LukA | 57 | GCAAACAAAGACTCACAAGATCAGACAAAGAAAGAGCA |
| W97 W92 | | TGTAGACAAAGCTCAACAGAAGGAAAAGCGCAATGTGA |
| | | ACGACAAGGATAAAAATACTCCTGGTCCAGATGACATTG |
| | | GTAAGAATGGTAAAGTTACTAAGCGGACCGTCTCTGAAT |
| | | ATGATAAGGAGACAAATATTCTCCAGAATTTGCAATTCG |
| | | ATTTCATTGATGATCCGACGTGCGATAAGAACGTATTGCT |
| | | CGTTAAAATGCAGGGCTCCATCCATTCGAATCTCAAGTTC |
| | | GAATCCCATCGCAACGAGACAAACGCTTCCTGGCTCAAA |
| | | TATCCTAGCGAGTATCATATCGACTTCCAAGTTCAACGGA |
| | | ACCCTAAAACTGAAATCCTTGATCAACTCCCTAAGAACA |
| | | AAATCTCAACTGCCAAGGTCTGTGCCACATTTTCTTATTC |
| | | TCTTGGCTGCAAATTCGATTCAACAAAGTGTATTGGTCGT |
| | | ACATCAAGTAATAGCTATAGTAAAAGCATCAGTTATAAC |
| | | CAGCAAAACTATGATACAATCGCGTCAGGCAAAAACAAT |
| | | AATCGTCATGTCCATTGGTCCATTGTCGCGAACGACCTTA |
| | | AGTACGGTAACGAAATTAAGAATCGGAACGATGAGTTTT |
| | | TGTTCTATCGCAACACCCGTCTGTCTACTGTCGAAAACCC |
| | | GGAGTTGTCCTTCGCAAGTAAATATCGCTATCCTGCTTTG |
| | | GTACGTTCTGGGTTTAACCCGGAATTTCTCGTCTACATCA |
| | | GCAACGAGAAAACAAATGACAAAACGCGCTTTGAAGTCA |
| | | CGTACACACGTAATCAGGACATCTTAAAAAATAAACCAG |
| | | GGATTCACTATGGTCAGCCAATCTTGGAGCAGAATAAAG |
| | | ACGGCCAGCGTTTCATTGTCGTTTATGAAGTGGACTGGAA |
| | | AAACAAAACTGTTAAGGTGGTTGAGAAATATTCCGACCA |
| | | AAACAAACCGTATAAGGCCGGT |
| CC8 LukB^{wt} | 58 | AAAATCAATTCTGAAATTAAGCAAGTGTCCGAAAAAAAT |
| | | TTGGATGGAGACACGAAGATGTATACGCGTACTGCTACG |
| | | ACGTCAGACTCCCAGAAGAACATTACACAGAGTCTGCAA |
| | | TTTAATTTTCTGACAGAACCAAACTATGACAAGGAAACT |
| | | GTCTTTATTAAGGCTAAAGGGACTATCGGAAGCGGCTTA |
| | | CGCATTTTAGACCCCAACGGTTATTGGAATAGCACGCTGC |
| | | GCTGGCCGGGCAGTTACTCAGTATCAATCCAAAATGTCG |
| | | ATGATAACAATAACACCAATGTTACCGATTTCGCCCCCAA |
| | | GAACCAGGATGAATCGCGCGAGGTTAAATACACATACGG |
| | | CTACAAGACAGGCGGTGACTTTAGCATCAACCGTGGGGG |
| | | CTTGACAGGGAATATTACTAAGGAATCAAATTATAGTGA |
| | | GACTATCTCTTATCAACAACCGTCCTATCGTACCTTATTA |
| | | GACCAGAGTACCTCCCACAAAGGTGTAGGGTGGAAAGTT |
| | | GAAGCGCACCTGATTAATAATATGGGTCACGATCACACA |
| | | CGCCAACTGACCAACGACAGTGACAACCGCACAAAAAGT |
| | | GAAATTTTTAGTCTTACCCGTAACGGAAATCTGTGGGCCA |
| | | AAGACAATTTTACACCGAAAGATAAGATGCCGGTCACTG |
| | | TATCTGAGGGGTTCAATCCCGAGTTTTTAGCAGTAATGTC |
| | | GCATGACAAAAAGGACAAAGGGAAATCCCAGTTTGTTGT |
| | | CCACTATAAGCGTAGCATGGATGAATTCAAAATCGACTG |
| | | GAACCGTCACGGTTTCTGGGGTTACTGGTCAGGTGAGAA |
| | | CCACGTAGACAAGAAAGAGGAGAAACTGAGCGCATTATA |
| | | TGAGGTTGATTGGAAAACGCACAATGTGAAATTTGTTAA |
| | | AGTCCTGAATGACAACGAGAAAAAG |
| CC45 LukB^{wt} | 59 | GAAATTAAGTCTAAGATCACAACAGTATCGGAGAAAAAC |
| | | CTGGATGGCGATACTAAGATGTATACACGCACCGCCACT |
| | | ACTTCGGACACGGAGAAGAAGATCTCACAATCGTTACAG |
| | | TTTAATTTTCTTACAGAACCGAACTACGACAAAGAGACC |
| | | GTCTTCATTAAAGCTAAAGGTACGATTGGTTCGGGATTAA |
| | | AAATTCTGAATCCGAATGGCTATTGGAACAGTACCTTACG |
| | | TTGGCCGGGGTCATATTCTGTATCCATTCAAAACGTGGAC |
| | | GACAATAACAACAGCACCAATGTGACAGATTTCGCTCCA |
| | | AAGAATCAGGATGAGTCCCGCGAGGTGAAATATACCTAT |
| | | GGGTACAAAACAGGAGGTGACTTTAGCATTAACCGTGGT |
| | | GGCTTGACTGGTAATATCACGAAGGAAAAAAATTACTCT |
| | | GAGACTATTTCCTACCAACAGCCGTCGTATCGCACCTTGA |
| | | TCGACCAACCAACGACTAACAAAGGGGTCGCGTGGAAAG |
| | | TTGAGGCCCACAGTATTAACAATATGGGCCACGATCACA |
| | | CTCGTCAGCTTACTAACGATTCGGATGACCGCGTCAAGTC |
| | | GGAAATTTTCAGCCTGACGCGTAACGGAAATTTGTGGGC |
| | | TAAAGACAATTTCACTCCTAAGAACAAGATGCCCGTGAC |
| | | TGTTTCCGAAGGCTTTAATCCCGAATTCTTAGCGGTGATG |
| | | TCTCATGATAAAAATGATAAAGGAAAATCGCGCTTCATT |
| | | GTGCATTATAAGCGTTCTATGGACGACTTCAAATTGGATT |
| | | GGAATAAGCACGGATTCTGGGGGTACTGGTCCGGGGAAA |
| | | ATCACGTAGATCAAAAGGAAGAGAAGTTGTCCGCTTTGT |
| | | ATGAAGTGGACTGGAAGACTCACGACGTTAAGTTGATCA |
| | | AGACCTTCAATGACAAAGAGAAGAAA |
| CC8 LukB | 60 | AAGATCAATTCGGAAATTAAACAGGTAAGTGAGAAAAAT |
| Val53Leu | | TTGGATGGCGATACCAAAATGTACACCCGCACCGCTACC |
| | | ACGTCAGATTCACAAAAAAATATTACACAGTCCTTGCAG |
| | | TTCAATTTCCTGACAGAACCGAATTACGACAAGGAGACT |
| | | TTGTTCATTAAAGCCAAGGGAACCATCGGGTCCGGATTG |
| | | CGTATCTTGGACCCGAACGGATATTGGAACTCGACCTTAC |
| | | GTTGGCCGGGGTCTTACAGTGTTAGTATCCAAAACGTAG |
| | | ATGATAACAATAACACAAACGTGACAGATTTTGCACCTA |
| | | AAAACCAGGACGAAAGCCGCGAGGTAAAGTACACATAT |
| | | GGGTATAAAACAGGGGGGGACTTTTCCATCAACCGTGGT |
| | | GGTTTGACCGGGAACATCACCAAAGAGTCAAATTACAGT |
| | | GAGACCATCAGTTATCAGCAGCCGTCCTATCGTACATTAT |
| | | TGGATCAGTCGACTTCACATAAAGGGGTCGGATGGAAAG |
| | | TAGAGGCTCATTTGATCAACAACATGGGTCACGATCATA |
| | | CACGTCAGTTAACGAACGATAGCGATAATCGCACGAAGT |
| | | CAGAAATCTTTAGTCTGACTCGTAACGGTAACTTGTGGGC |
| | | CAAGGACAATTTCACGCCCAAAGATAAGATGCCTGTGAC |
| | | GGTATCGGAGGGGTTCAATCCAGAATTCCTTGCTGTAATG |
| | | TCCCATGACAAAAAAGACAAGGGCAAATCGCAATTTGTA |
| | | GTCCACTATAAGCGTTCTATGGACGAGTTCAAGATTGACT |
| | | GGAACCGCCACGGCTTCTGGGGGTACTGGAGTGGTGAGA |
| | | ATCATGTGGATAAAAAGGAGGAGAAACTTAGCGCCCTGT |
| | | ATGAGGTAGATTGGAAAACACACAATGTCAAGTTCGTGA |
| | | AAGTTCTTAATGACAACGAAAAAAAA |
| CC45 LukB | 61 | GAGATCAAGAGCAAAATTACCACCGTGAGCGAAAAGAA |
| Val53Leu | | CCTGGACGGTGATACCAAAATGTATACCCGTACCGCGAC |
| | | CACCAGCGACACCGAGAAGAAAATTAGCCAGAGCCTGCA |
| | | ATTCAACTTTCTGACCGAGCCGAACTACGATAAGGAAAC |
| | | CCTGTTCATCAAGGCGAAAGGCACCATTGGTAGCGGCCT |
| | | GAAAATCCTGAACCCGAACGGTTATTGGAACAGCACCCT |
| | | GCGTTGGCCGGGTAGCTACAGCGTGAGCATCCAGAACGT |
| | | TGACGATAACAACAACAGCACCAACGTGACCGACTTCGC |
| | | GCCGAAGAACCAAGATGAGAGCCGTGAAGTTAAATACAC |
| | | CTATGGTTACAAAACCGGTGGCGACTTTAGCATTAACCGT |
| | | GGTGGCCTGACCGGCAACATCACCAAGGAGAAAAACTAT |
| | | AGCGAAACCATTAGCTATCAGCAACCGAGCTACCGTACC |
| | | CTGATCGATCAGCCGACCACCAACAAGGGTGTGGCGTGG |
| | | AAAGTTGAGGCGCACAGCATTAACAACATGGGCCACGAC |
| | | CACACCCGTCAACTGACCAACGATAGCGACGATCGTGTG |
| | | AAGAGCGAAATCTTCAGCCTGACCCGTAACGGTAACCTG |
| | | TGGGCGAAAGACAACTTTACCCCGAAGAACAAAATGCCG |
| | | GTGACCGTTAGCGAGGGTTTCAACCCGGAATTTCTGGCG |
| | | GTGATGAGCCACGACAAGAACGATAAGGGCAAAAGCCG |
| | | TTTCATTGTTCACTACAAACGTAGCATGGACGATTTCAAG |
| | | CTGGACTGGAACAAACACGGTTTTTGGGGCTATTGGAGC |
| | | GGCGAGAACCACGTTGATCAGAAAGAGGAGAAACTGAG |
| | | CGCGCTGTACGAAGTGGACTGGAAGACCCACGATGTTAA |
| | | GCTGATCAAAACCTTTAACGATAAAGAAAAGAAA |

In any embodiment, the nucleic acid molecules encoding the variant LukA and LukB polypeptide as described herein are codon optimized for expression in mammalian cells, preferably human cells. Methods of codon-optimization are known and have been described previously (e.g. International Patent Application Publication No. WO1996/09378 to Seed, which is hereby incorporated by reference in its entirety). A sequence is considered codon optimized if at least one non-preferred codon as compared to a wild-type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables that are well known and available in the art. Preferably more than one non-preferred codon, e.g. more than 10%, 40%, 60%, 80% of non-preferred codons, preferably most (e.g. at least 90%) or all non-preferred codons, are replaced by codons that are more preferred. Preferably the most frequently used codons in an organism are used in a codon-optimized sequence. Replacement by preferred codons generally leads to higher expression.

Polynucleotide sequences of the present disclosure can be cloned using routine molecular biology techniques, or generated de novo by DNA synthesis, which can be performed using routine procedures by service companies having business in the field of DNA synthesis and/or molecular cloning (e.g. GeneArt, GenScript, Invitrogen, Eurofins).

In any embodiment, the aforementioned nucleic acid molecules are inserted into a vector, i.e., an expression vector for use in a vaccine composition as described herein. Alternatively, these nucleic acid molecules may be inserted into an expression vector that is transformed or transfected into an appropriate host cell for expression and isolation of the encoded variant LukA protein, variant LukB protein, or variant LukAB complex (as a stable heterodimer), where the variant LukAB complex comprises a variant LukA and non-variant LukB, a non-variant LukA and variant LukB, or a variant LukA and variant LukB as disclosed herein.

In accordance with this aspect of the disclosure, the nucleic acid molecules encoding the S. aureus LukA and/or LukB proteins and polypeptides thereof as described herein can be incorporated into any expression vector capable of expressing the LukA and/or LukB proteins or polypeptides encoded by the nucleic acid sequence construct. Suitable expression vectors comprise nucleic acid sequence elements that control, regulate, cause or permit expression of the LukA and/or LukB protein or polypeptide encoded by such a vector. Such elements may comprise transcriptional enhancer binding sites, RNA polymerase initiation sites, ribosome binding sites, and other sites that facilitate the expression of encoded polypeptides in a given expression system. Suitable vectors include, without limitation, DNA vectors, plasmid vectors, a linear nucleic acid, and a viral vector, e.g., adenoviral vectors.

In one embodiment, the expression vector is a circular plasmid (see, e.g., Muthumani et al., “Optimized and Enhanced DNA Plasmid Vector Based In vivo Construction of a Neutralizing anti-HIV-1 Envelope Glycoprotein Fab,” Hum. Vaccin. Immunother. 9: 2253-2262 (2013), which is hereby incorporated by reference in its entirety). Plasmids can transform a target cell by integration into the cellular genome or exist extrachromosomally (e.g., autonomous replicating plasmid with an origin of replication). Exemplary plasmid vectors include, without limitation, pCEP4, pREP4, pVAX, pcDNA3.0, provax, or any other plasmid expression vector capable of expressing the variant LukA and/or variant LukB proteins or polypeptides encoded by the recombinant nucleic acid sequence construct.

In another embodiment, the expression vector is a linear expression cassette (“LEC”). LECs are capable of being efficiently delivered to a subject via electroporation to express the LukA and/or LukB proteins or polypeptides encoded by the recombinant nucleic acid molecules described herein. The LEC may be any linear DNA devoid of a phosphate backbone. In one embodiment, the LEC does not contain any antibiotic resistance genes and/or a phosphate backbone. In another embodiment, the LEC does not contain other nucleic acid sequences unrelated to the desired gene expression.

The LEC may be derived from any plasmid capable of being linearized. The plasmid may be capable of expressing the LukA and/or LukB proteins or polypeptides encoded by the recombinant nucleic acid molecules as described herein. Exemplary plasmids include, without limitation, pNP (Puerto Rico/34), pM2 (New Caledonia/99), WLV009, pVAX, pcDNA3.0, or provax, or any other expression vector capable of expressing the variant LukA and/or variant LukB proteins or polypeptides encoded by the recombinant nucleic acid sequence construct.

In another embodiment, the expression vector is a viral vector. Suitable viral vectors that are capable of expressing the LukA and/or t LukB proteins or polypeptides include, for example, an adeno-associated virus (AAV) vector (see, e.g., Krause et al., “Delivery of Antigens by Viral Vectors for Vaccination,” Ther. Deliv. 2(1):51-70 (2011); Ura et al., “Developments in Viral Vector-Based Vaccines,” Vaccines 2: 624-641 (2014); Buning et al, “Recent Developments in Adeno-associated Virus Vector Technology,” J. Gene Med. 10:717-733 (2008), each of which is incorporated herein by reference in its entirety), a lentivirus vector (see, e.g., Ura et al., “Developments in Viral Vector-Based Vaccines,” Vaccines 2: 624-641 (2014); and Hu et al., “Immunization Delivered by Lentiviral Vectors for Cancer and Infection Diseases,” Immunol. Rev. 239: 45-61 (2011), which are hereby incorporated by reference in their entirety), a retrovirus vector (see e.g., Ura et al., “Developments in Viral Vector-Based Vaccines,” Vaccines 2: 624-641 (2014), which are hereby incorporated by reference in their entirety), a vaccinia virus, a replication deficient adenovirus vector, and a gutless adenovirus vector (see e.g., U.S. Pat. No. 5,872,005, which is incorporated herein by reference in its entirety). Methods for generating and isolating adeno-associated viruses (AAVs) suitable for use as vectors are known in the art (see, e.g., Grieger & Samulski, “Adeno-associated Virus as a Gene Therapy Vector: Vector Development, Production and Clinical Applications,” Adv. Biochem. Engin Biotechnol. 99: 119-145 (2005); Buning et al, “Recent Developments in Adeno-associated Virus Vector Technology,” J. Gene Med. 10:717-733 (2008), each of which is incorporated herein by reference in its entirety).

The nucleic acid molecules encoding the LukA and/or LukB proteins or polypeptides described herein are typically combined with sequences of a promoter, translation initiation, 3′ untranslated region, polyadenylation, and transcription termination in the expression vector constructs to achieve maximal expression. Promoter sequences suitable for driving expression of the LukA and/or LukB proteins or polypeptides thereof include, without limitation, the elongation factor 1-alpha (EF1a) promoter, a phosphoglycerate kinase-1 (PGK) promoter, a cytomegalovirus immediate early gene promoter (CMV), a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter. Other promoters suitable for driving gene expression in host cells that are known in the art are also suitable for incorporation into the expression constructs disclosed herein.

Another aspect of the present disclosure is directed to a host cell comprising a vector containing a polynucleotide encoding the LukA and/or LukB polypeptides as described herein. Expression constructs encoding the LukA and LukB proteins or polypeptides as described herein can be co-transfected, serially transfected, or separately transfected into host cells. The LukA and LukB proteins and polypeptides as described herein can optionally be produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety). Such host cells may be eukaryotic cells, bacterial cells, plant cells or archaeal cells.

In any embodiment, the LukA and/or LukB polypeptides as described herein are produced in a bacterial cell. Suitable bacterial host cells include, without limitation, Escherichia host cells, Pseudomonas host cells, Staphylococcus host cells, Streptomyces host cells, Mycobacterium host cells, and Bacillus host cells. In any embodiment, the host cell is an Escherichia coli host cell. In any embodiment, the host cell is a S. aureus host cell.

In any embodiment, the LukA and/or LukB polypeptides as described herein are produced in a eukaryotic cell. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, Va., CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics, Walkersville, Md.), CHO-K1 (ATCC CRL-61) or DG44.

The LukA and LukB polypeptides as described herein can be prepared by any of a variety of techniques using the isolated polynucleotides, vectors, and host cells described supra. In general, proteins are produced by standard cloning and cell culture techniques commonly used to prepare the recombinant expression vector, transfect the host cells, select for transformants, culture the host cells, and recover the proteins or polypeptides from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like.

The LukA and/or LukB polypeptides described herein can be post-translationally modified by processes such as glycosylation, isomerization, deglycosylation or non-naturally occurring covalent modification such as the addition of polyethylene glycol (PEG) moieties (pegylation) and lipidation. Such modifications may occur in vivo or in vitro.

In any embodiment, the LukA and LukB polynucleotides and/or polypeptides as described herein are preferably “isolated” polynucleotides and/or polypeptides. “Isolated” when used to describe the polynucleotides and/or polypeptides disclosed herein, means that the polynucleotides and/or polypeptides has been identified, separated and/or recovered from a component of its production environment. Preferably, the isolated polynucleotides and/or polypeptides is free of association with other components from its production environment. Contaminant components of its production environment, such as that resulting from recombinant transfected cells, are materials that could typically interfere with pharmaceutical use, and may include enzymes, hormones, and other proteinaceous or non-proteinaceous solutes. The polynucleotides and/or polypeptides are recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. High performance liquid chromatography (“HPLC”) can also be used for purification.

S. aureus Vaccine Compositions

Another aspect of the present disclosure is directed to Staphylococcus aureus vaccine compositions. In any embodiment, the S. aureus vaccine composition comprises any one or more of the LukA variant polypeptides as described herein, or one or more nucleic acid molecules encoding the LukA variant polypeptides described herein. In particular, the LukA variant polypeptide of the vaccine composition comprises an amino acid residue insertion, substitution, and/or deletion at any of the one or more amino acid residues as identified and described herein. In any embodiment, the LukA variant of the vaccine composition comprises a variant of SEQ ID NO: 25 or a variant of any one of SEQ ID NOs: 1, 2, or 26-38. In any embodiment, the LukA variant of the vaccine composition comprises a variant of SEQ ID NO: 1 (CC8). Exemplary CC8 LukA variants include, without limitation, the LukA variants of SEQ ID NOs: 3, 5, 7, 9, and 13. In any embodiment, the LukA variant of the vaccine composition comprises a variant of SEQ ID NO: 2 (CC45). Exemplary CC45 LukA variants include, without limitation, the LukA variants of SEQ ID NOs: 4, 6, 8, 10, 11, 12, and 14.

In any embodiment, the S. aureus vaccine composition disclosed herein comprises a CC8 LukA variant having the amino acid sequence of SEQ ID NO: 3.

In any embodiment, the S. aureus vaccine composition disclosed herein comprises a CC8 LukA variant having the amino acid sequence of SEQ ID NO: 7.

In any embodiment, the S. aureus vaccine composition disclosed herein comprises a CC45 LukA variant having the amino acid sequence of SEQ ID NO: 8.

In any embodiment, the S. aureus vaccine composition disclosed herein comprise any one or more of the variant LukB proteins or polypeptides as described herein, or one or more nucleic acid molecules encoding the LukB variant proteins or polypeptides described herein. In particular, the LukB variant polypeptide of the vaccine composition comprises an amino acid residue insertion, substitution, and/or deletion at any of the one or more amino acid residues as identified and described herein. In any embodiment, the LukB variant of the vaccine composition comprises a variant of SEQ ID NO: 39 or a variant of any one of SEQ ID NOs: 15, 16, or 40-51. In any embodiment, the LukB variant of the vaccine composition comprises a variant of SEQ ID NO: 15 (CC8). Exemplary CC8 LukB variants include, without limitation, the LukB variants of SEQ ID NOs: 17, 19, and 21. In any embodiment, the LukB variant of the vaccine composition comprises a variant of SEQ ID NO: 16 (CC45). Exemplary CC45 LukB variants include, without limitation, the LukB variants of SEQ ID NOs: 18, 20, and 21.

In any embodiment, the vaccine composition as disclosed herein comprises both LukA and LukB proteins. Accordingly, in any embodiment, the vaccine composition comprises a CC8 LukA variant of SEQ ID NO:1 in combination with a CC8 LukB non-variant sequence of SEQ ID NO: 15 or a variant sequence having at least 85%, 86%, 87%, 88%, 89%, 90%, 91%, 92%, 93%, 94%, 95%, 96%, 97%, 98%, or 99% sequence identity to SEQ ID NO: 15. In any embodiment, the CC8 LukB sequence variant sequence comprises an amino acid sequence selected from SEQ ID NOs: 17, 19 and 21. For example, in any embodiment, the vaccine composition comprises a CC8 LukA variant of SEQ ID NO: 3 in combination with a CC8 LukB sequence of SEQ ID NO: 15 or a variant thereof having 85% or more sequence identity to CC8 LukB of SEQ ID NO: 15, e.g., a CC8 LukB variant sequence selected from SEQ ID NOs: 17, 19 and 21. In any embodiment, the vaccine composition comprises a CC8 LukA variant of SEQ ID NO: 5 in combination with a CC8 LukB sequence of SEQ ID NO: 15 or a variant thereof having >85% sequence identity to CC8 LukB of SEQ ID NO: 15, e.g., a CC8 LukB variant sequence selected from SEQ ID NOs: 17, 19 and 21. In any embodiment, the vaccine composition comprises a CC8 LukA variant of SEQ ID NO: 7 in combination with a CC8 LukB sequence of SEQ ID NO: 15 or a variant thereof having >85% sequence identity to CC8 LukB of SEQ ID NO: 15, e.g., a CC8 LukB variant sequence selected from SEQ ID NOs: 17, 19 and 21. In any embodiment, the vaccine composition comprises a CC8 LukA variant of SEQ ID NO: 9 in combination with a CC8 LukB sequence of SEQ ID NO: 15 or a variant thereof having >85% sequence identity to CC8 LukB of SEQ ID NO: 15, e.g., a CC8 LukB variant sequence selected from SEQ ID NOs: 17, 19 and 21.

In any embodiment, the vaccine composition comprises a CC8 LukA variant having the amino acid sequence of SEQ ID NO: 3 and a CC8 LukB variant having the amino acid sequence of SEQ ID NO: 15.

In any embodiment, the vaccine composition comprises a CC8 LukA variant having the amino acid sequence of SEQ ID NO: 3 and a CC8 LukB variant having the amino acid sequence of SEQ ID NO: 17.

In any embodiment, the vaccine composition comprises a CC8 LukA variant of SEQ ID NO:1 in combination with a CC45 LukB sequence of SEQ ID NO: 16 or a variant sequence having at least 85%, 86%, 87%, 88%, 89%, 90%, 91%, 92%, 93%, 94%, 95%, 96%, 97%, 98%, or 99% sequence identity to SEQ ID NO: 16. In any embodiment, the CC45 LukB variant sequence comprises the amino acid sequence selected from SEQ ID NOs: 18, 20, and 22. For example, in any embodiment, the vaccine composition comprises a CC8 LukA variant of SEQ ID NO: 3 in combination with a CC45 LukB sequence of SEQ ID NO: 16 or a variant thereof having >85% sequence identity to CC45 LukB of SEQ ID NO: 16, e.g., a CC45 LukB variant sequence selected from SEQ ID NOs: 18, 20, and 22. In any embodiment, the vaccine composition comprises a CC8 LukA variant of SEQ ID NO: 5 in combination with a CC45 LukB sequence of SEQ ID NO: 16 or a variant thereof having >85% sequence identity to CC45 LukB of SEQ ID NO: 16, e.g., a CC45 LukB variant sequence selected from SEQ ID NOs: 18, 20, and 22. In any embodiment, the vaccine composition comprises a CC8 LukA variant of SEQ ID NO: 7 in combination with a CC45 LukB sequence of SEQ ID NO: 16 or a variant thereof having >85% sequence identity to CC45 LukB of SEQ ID NO: 16, e.g., a CC45 LukB variant sequence selected from SEQ ID NOs: 18, 20, and 22. In any embodiment, the vaccine composition comprises a CC8 LukA variant of SEQ ID NO: 9 in combination with a CC45 LukB sequence of SEQ ID NO: 16 or a variant thereof having >85% sequence identity to CC45 LukB of SEQ ID NO: 16, e.g., a CC45 LukB variant sequence selected from SEQ ID NOs: 18, 20, and 22.

In any embodiment, the vaccine composition comprises a CC8 LukA variant having the amino acid sequence of SEQ ID NO: 5 and a CC45 LukB variant having the amino acid sequence of SEQ ID NO: 16.

In any embodiment, the vaccine composition comprises a CC8 LukA variant having the amino acid sequence of SEQ ID NO: 5 and a CC45 LukB variant having the amino acid sequence of SEQ ID NO: 22.

In any embodiment, the vaccine composition comprises a CC8 LukA variant having the amino acid sequence of SEQ ID NO: 5 and a CC45 LukB variant having the amino acid sequence of SEQ ID NO: 18.

In any embodiment, the vaccine composition comprises a CC8 LukA variant having the amino acid sequence of SEQ ID NO: 5 and a CC45 LukB variant having the amino acid sequence of SEQ ID NO: 20.

In any embodiment, the vaccine compositions comprise the variant LukA protein comprising the amino acid sequence SEQ ID NO: 3 and the LukB protein comprising the amino acid sequence of SEQ ID NO:18

In any embodiment, the vaccine composition comprises a CC45 LukA variant of SEQ ID NO:2 in combination with a CC45 LukB sequence of SEQ ID NO: 16 or a variant sequence having at least 85%, 86%, 87%, 88%, 89%, 90%, 91%, 92%, 93%, 94%, 95%, 96%, 97%, 98%, or 99% sequence identity to SEQ ID NO: 16. In any embodiment, the CC45 LukB variant sequence comprises the amino acid sequence selected from SEQ ID NOs: 18, 20, and 22. For example, in any embodiment, the vaccine composition comprises a CC45 LukA variant of SEQ ID NO: 4 in combination with a CC45 LukB sequence of SEQ ID NO: 16 or a variant thereof having >85% sequence identity to CC45 LukB of SEQ ID NO: 16, e.g., a CC45 LukB variant sequence selected from SEQ ID NOs: 18, 20, and 22. In any embodiment, the vaccine composition comprises a CC45 LukA variant of SEQ ID NO: 6 in combination with a CC45 LukB sequence of SEQ ID NO: 16 or a variant thereof having >85% sequence identity to CC45 LukB of SEQ ID NO: 16, e.g., a CC45 LukB variant sequence selected from SEQ ID NOs: 18, 20, and 22. In any embodiment, the vaccine composition comprises a CC45 LukA variant of SEQ ID NO: 8 in combination with a CC45 LukB sequence of SEQ ID NO: 16 or a variant thereof having >85% sequence identity to CC45 LukB of SEQ ID NO: 16, e.g., a CC45 LukB variant selected from SEQ ID NOs: 18, 20, and 22. In any embodiment, the vaccine composition comprises a CC45 LukA variant of SEQ ID NO: 10 in combination with a CC45 LukB sequence of SEQ ID NO: 16 or a variant thereof having >85% sequence identity to CC45 LukB of SEQ ID NO: 16, e.g., a CC45 LukB variant selected from SEQ ID NOs: 18, 20, and 22. In any embodiment, the vaccine composition comprises a CC45 LukA variant of SEQ ID NO: 11 in combination with a CC45 LukB sequence of SEQ ID NO: 16 or a variant thereof having >85% sequence identity to CC45 LukB of SEQ ID NO: 16, e.g., a CC45 LukB variant selected from SEQ ID NOs: 18, 20, and 22. In any embodiment, the vaccine composition comprises a CC45 LukA variant of SEQ ID NO: 12 in combination with a CC45 LukB sequence of SEQ ID NO: 16 or a variant thereof having >85% sequence identity to CC45 LukB of SEQ ID NO: 16, e.g., a CC45 LukB variant selected from SEQ ID NOs: 18, 20, and 22.

In one embodiment, the vaccine composition comprises a CC45 LukA variant having an amino acid sequences of SEQ ID NO: 4 in combination with a CC45 LukB having the amino acid sequence of SEQ ID NO: 16.

In one embodiment, the vaccine composition comprises a CC45 LukA variant having an amino acid sequences of SEQ ID NO: 11 in combination with a CC45 LukB having the amino acid sequence of SEQ ID NO: 16.

In one embodiment, the vaccine composition comprises a CC45 LukA variant having an amino acid sequences of SEQ ID NO: 12 in combination with a CC45 LukB having the amino acid sequence of SEQ ID NO: 16.

In one embodiment, the vaccine composition comprises a CC45 LukA variant having an amino acid sequences of SEQ ID NO: 8 in combination with a CC45 LukB having the amino acid sequence of SEQ ID NO: 16.

In one embodiment, the vaccine composition comprises a CC45 LukA variant having an amino acid sequences of SEQ ID NO: 4 in combination with a CC45 LukB variant having the amino acid sequence of SEQ ID NO: 18.

In any embodiment, the vaccine composition comprises a CC45 LukA variant of SEQ ID NO:2 in combination with a CC8 LukB sequence of SEQ ID NO: 15 or a variant sequence having at least 85%, 86%, 87%, 88%, 89%, 90%, 91%, 92%, 93%, 94%, 95%, 96%, 97%, 98%, or 99% sequence identity to SEQ ID NO: 15. In any embodiment, the CC8 LukB variant sequence comprises the amino acid sequence selected from SEQ ID NOs: 17, 19 and 21. For example, in any embodiment, the vaccine composition comprises a CC45 LukA variant of SEQ ID NO: 4 in combination with a CC8 LukB sequence of SEQ ID NO: 15 or a variant thereof having >85% sequence identity to CC8 LukB sequence of SEQ ID NO: 15, e.g., a variant sequence selected from SEQ ID NOs: 17, 19 and 21. In any embodiment, the vaccine composition comprises a CC45 LukA variant of SEQ ID NO: 6 in combination with a CC8 LukB sequence of SEQ ID NO: 15 or a variant thereof having >85% sequence identity to CC8 LukB sequence of SEQ ID NO: 15, e.g., a variant sequence selected from SEQ ID NOs: 17, 19 and 21. In any embodiment, the vaccine composition comprises a CC45 LukA variant of SEQ ID NO: 8 in combination with a CC8 LukB sequence of SEQ ID NO: 15 or a variant thereof having >85% sequence identity to CC8 LukB sequence of SEQ ID NO: 15, e.g., a variant sequence selected from SEQ ID NOs: 17, 19 and 21. In any embodiment, the vaccine composition comprises a CC45 LukA variant of SEQ ID NO: 9 in combination with a CC8 LukB sequence of SEQ ID NO: 15 or a variant thereof having >85% sequence identity to CC8 LukB sequence of SEQ ID NO: 15, e.g., a variant sequence selected from SEQ ID NOs: 17, 19 and 21. In any embodiment, the vaccine composition comprises a CC45 LukA variant of SEQ ID NO: 10 in combination with a CC8 LukB sequence of SEQ ID NO: 15 or a variant thereof having >85% sequence identity to CC8 LukB sequence of SEQ ID NO: 15, e.g., a variant sequence selected from SEQ ID NOs: 17, 19 and 21. In any embodiment, the vaccine composition comprises a CC45 LukA variant of SEQ ID NO: 11 in combination with a CC8 LukB sequence of SEQ ID NO: 15 or a variant thereof having >85% sequence identity to CC8 LukB sequence of SEQ ID NO: 15, e.g., a variant sequence selected from SEQ ID NOs: 17, 19 and 21. In any embodiment, the vaccine composition comprises a CC45 LukA variant of SEQ ID NO: 12 in combination with a CC8 LukB sequence of SEQ ID NO: 15 or a variant thereof having >85% sequence identity to CC8 LukB sequence of SEQ ID NO: 15, e.g., a variant sequence selected from SEQ ID NOs: 17, 19 and 21.

Another aspect of the present disclosure is directed to a Staphylococcus aureus vaccine composition comprising any of the variant LukB variant polypeptides as described herein or a nucleic acid molecule encoding the LukB variant. In particular, the LukB variant polypeptide of the vaccine composition comprises one or more amino acid residue insertions, substitutions, and/or deletions described herein. In any embodiment, the LukB variant of the vaccine composition comprises a variant of SEQ ID NO: 15 (CC8). Exemplary CC8 LukB variants include, without limitation, the LukB variants of SEQ ID NOs: 17, 19, and 21. In any embodiment, the LukB variant of the vaccine composition comprises a variant of SEQ ID NO: 16 (CC45). Exemplary CC45 LukB variants include, without limitation, the LukB variants of SEQ ID NOs: 18, 20, and 22.

In any embodiment, the vaccine composition as disclosed herein comprises a LukB variant polypeptide as described herein with a LukA protein or polypeptide. Accordingly, in any embodiment, the vaccine composition comprises a CC8 LukB variant of SEQ ID NO:15, e.g., a variant of SEQ ID NOs: 17, 19, and 21 in combination with a CC8 LukA sequence of SEQ ID NO: 1 or a variant sequence having at least 85%, 86%, 87%, 88%, 89%, 90%, 91%, 92%, 93%, 94%, 95%, 96%, 97%, 98%, or 99% sequence identity to SEQ ID NO: 1. In any embodiment, the vaccine composition comprises a CC8 LukB variant of SEQ ID NO:15, e.g., a variant of SEQ ID NOs: 17, 19, and 21, in combination with a CC45 LukA sequence of SEQ ID NO: 2 or a variant sequence having at least 85%, 86%, 87%, 88%, 89%, 90%, 91%, 92%, 93%, 94%, 95%, 96%, 97%, 98%, or 99% sequence identity to SEQ ID NO: 2.

In any embodiment, the vaccine composition comprises a CC45 LukB variant of SEQ ID NO:16, e.g., a variant of SEQ ID NO: 18, 20, or 22, in combination with a CC8 LukA sequence of SEQ ID NO: 1 or a variant sequence having at least 85%, 86%, 87%, 88%, 89%, 90%, 91%, 92%, 93%, 94%, 95%, 96%, 97%, 98%, or 99% sequence identity to SEQ ID NO: 1. In any embodiment, the vaccine composition comprises a CC45 LukB variant of SEQ ID NO: 16, e.g., a variant of SEQ ID NO: 18, 20, or 22 in combination with a CC45 LukA sequence of SEQ ID NO: 2 or a variant sequence having at least 85%, 86%, 87%, 88%, 89%, 90%, 91%, 92%, 93%, 94%, 95%, 96%, 97%, 98%, or 99% sequence identity to SEQ ID NO: 2.

The vaccine compositions of the present disclosure are prepared by formulating the LukA and/or LukB polypeptides as described herein with a pharmaceutically acceptable carrier and optionally a pharmaceutically acceptable excipient. The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier can be used in formulating the pharmaceutical compositions of the invention.

As used herein, the terms “pharmaceutically acceptable carrier” and “pharmaceutically acceptable excipient” (e.g., additives such as diluents, immunostimulants, adjuvants, antioxidants, preservatives and solubilizing agents) are non-toxic to the subject administered the composition at the dosages and concentrations employed. Examples of pharmaceutically acceptable carriers include water, e.g., buffered with phosphate, citrate and another organic acid. Representative examples of pharmaceutically acceptable excipients that may be useful in the present disclosure include antioxidants such as ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants.

In any embodiment, the vaccine composition as described herein is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

The vaccine composition may further comprise one or more adjuvants. As used herein, the term “adjuvant” refers to a compound that when administered in conjunction with the LukA and/or LukB polypeptides described herein augments, enhances, and/or boosts the immune response to the polypeptides. However, when the adjuvant compound is administered alone it does not generate an immune response to the aforementioned polypeptides or polynucleotides encoding the same. Adjuvants can enhance an immune response by several mechanisms including, e.g., lymphocyte recruitment, stimulation of B and/or T cells, and stimulation of antigen presenting cells.

The vaccine composition described herein comprising the LukA and/or LukB polypeptides and/or polynucleotides encoding the same, comprises an adjuvant or is administered in combination with an adjuvant. The adjuvant for administration in combination with the vaccine composition described herein can be administered before, concomitantly with, or after administration of the vaccine composition.

Suitable adjuvants are known in the art and include, without limitation, flagellin, Freund's complete adjuvant, Freund's incomplete adjuvant, aluminum hydroxide, lysolecithin, pluronic polyols, polyanions, peptides, oil emulsion, dinitrophenol, iscomatrix, and liposome polycation DNA particles. Additional examples of adjuvants include, for example, β-glucan as described in U.S. Pat. No. 6,355,625, which is hereby incorporated by reference in its entirety, or a granulocyte colony stimulating factor (GCSF).

Additional exemplary adjuvants include, but are not limited to, aluminum salts (alum) (such as aluminum hydroxide, aluminum phosphate, aluminum sulfate, and aluminum oxide, including nanoparticles comprising alum or nanoalum formulations), calcium phosphate (e.g., Masson J D et al, Expert Rev Vaccines 16: 289-299 (2017), which is hereby incorporated by reference in its entirety), monophosphoryl lipid A (MPL) or 3-de-O-acylated monophosphoryl lipid A (3D-MPL) (see e.g., United Kingdom Patent GB2220211, EP0971739, EP 1194166, U.S. Pat. No. 6,491,919, which are hereby incorporated by reference in their entirety), AS01, AS02, AS03 and AS04 (see e.g. EP 1126876, U.S. Pat. No. 7,357,936 for AS04, EP0671948, EP0761231, U.S. Pat. No. 5,750,110 for AS02, which are hereby incorporated by reference in their entirety), imidazopyridine compounds (see WO2007/109812, which is hereby incorporated by reference in its entirety), imidazoquinoxaline compounds (see WO2007/109813, which is hereby incorporated by reference in its entirety), delta-inulin (e.g. Petrovsky N and PD Cooper, Vaccine 33: 5920-5926 (2015), which is hereby incorporated by reference in its entirety), STING-activating synthetic cyclic-di-nucleotides (e.g. US20150056224, which is hereby incorporated by reference in its entirety), combinations of lecithin and carbomer homopolymers (e.g. U.S. Pat. No. 6,676,958, which is hereby incorporated by reference in its entirety), and saponins, such as Quil A and QS21 (see e.g. Zhu D and W Tuo, 2016, Nat Prod Chem Res 3: e113 (doi:10.4172/2329-6836.1000e113), which is hereby incorporated by reference in its entirety), optionally in combination with QS7 (see Kensil et al., in Vaccine Design: The Subunit and Adjuvant Approach (eds. Powell & Newman, Plenum Press, N Y, 1995); U.S. Pat. No. 5,057,540, which are hereby incorporated by reference in their entirety). In any embodiment, the adjuvant is Freund's adjuvant (complete or incomplete). In any embodiment, the adjuvant comprises Quil-A, such as for instance commercially obtainable from Brenntag (now Croda) or Invivogen. QuilA contains the water-extractable fraction of saponins from the Quillaja saponaria Molina tree. These saponins belong to the group of triterpenoid saponins, that have a common triterpenoid backbone structure. Saponins are known to induce a strong adjuvant response to T-dependent as well as T-independent antigens, as well as strong cytotoxic CD8+ lymphocyte responses and potentiating the response to mucosal antigens. Accordingly, in any embodiment, the adjuvant comprises saponins. In any embodiment, the adjuvant comprises QS-21.

In any embodiment, the saponin is combined with cholesterol and phospholipids, to form immunostimulatory complexes (ISCOMs), that can activate both antibody-mediated and cell-mediated immune responses to a broad range of antigens from different origins. In certain embodiments, the adjuvant is AS01, for example AS01B. AS01 is an adjuvant system containing MPL (3-O-desacyl-4′-monophosphoryl lipid A), QS21 (Quillaja saponaria Molina, fraction 21), and liposomes. In certain embodiments, the AS01 is commercially available or can be made as described in WO 96/33739, which is hereby incorporated by reference in its entirety. Certain adjuvants comprise emulsions, which are mixtures of two immiscible fluids, e.g. oil and water, one of which is suspended as small drops inside the other and are stabilized by surface-active agents. Oil-in-water emulsions have water forming the continuous phase, surrounding small droplets of oil, while water-in-oil emulsions have oil forming the continuous phase. Certain oil-in-water emulsions comprise squalene (a metabolizable oil). Certain adjuvants comprise block copolymers, which are copolymers formed when two monomers cluster together and form blocks of repeating units. An example of a water in oil emulsion comprising a block copolymer, squalene and a microparticulate stabilizer is TiterMax®, which can be commercially obtained from Sigma-Aldrich.

Optionally, emulsions can be combined with or comprise further immunostimulating components, such as a TLR4 agonist. Suitable, but non-limiting examples of adjuvant combinations for use in the compositions disclosed herein include, oil in water emulsions (such as squalene or peanut oil), MF59 (see e.g. EP0399843, U.S. Pat. Nos. 6,299,884, 6,451,325, which are hereby incorporated by reference in their entirety), and AS03, optionally in combination with immune stimulants, such as monophosphoryl lipid A and/or QS21 such as in AS02 (see Stoute et al., N. Engl. J. Med. 336: 86-91 (1997)s, which is hereby incorporated by reference in its entirety). Further examples of adjuvants are liposomes containing immune stimulants such as MPL and QS21, such as in AS01E and AS01B (see e.g., US 2011/0206758, which is hereby incorporated by reference in its entirety). Other examples of adjuvants are CpG and imidazoquinolines (such as imiquimod and R848) (see e.g., Reed G. et al., Nature Med, 19: 1597-1608 (2013), which is hereby incorporated by reference in its entirety). In any embodiment according to the invention, the adjuvant is a Th1 adjuvant.

In any embodiment, the adjuvant of the vaccine composition disclosed herein contains a toll-like receptor 4 (TLR4) agonist alone or in combination with another adjuvant. TLR4 agonists are well known in the art, see e.g. Ireton GC and SG Reed, Expert Rev Vaccines 12: 793-807 (2013), which is hereby incorporated by reference in its entirety. In any embodiment, the adjuvant is a TLR4 agonist comprising lipid A, or an analog or derivative thereof.

In any embodiment, the adjuvant of the vaccine composition contains lipid A or a lipid A analog or derivative. As used herein, the term “lipid A” refers to the hydrophobic lipid moiety of an LPS molecule that comprises glucosamine and is linked to keto-deoxyoctulosonate in the inner core of the LPS molecule through a ketosidic bond, which anchors the LPS molecule in the outer leaflet of the outer membrane of Gram-negative bacteria. Lipid A, as used herein includes naturally occurring lipid A, mixtures, analogs, derivatives and precursors thereof. The term includes monosaccharides, e.g., the precursor of lipid A referred to as lipid X; disaccharide lipid A; hepta-acyl lipid A; hexa-acyl lipid A; penta-acyl lipid A; tetra-acyl lipid A, e.g., tetra-acyl precursor of lipid A, referred to as lipid IVA; dephosphorylated lipid A; monophosphoryl lipid A; diphosphoryl lipid A, such as lipid A from Escherichia coli and Rhodobacter sphaeroides. Several immune activating lipid A structures contain 6 acyl chains. Four primary acyl chains attached directly to the glucosamine sugars are 3-hydroxy acyl chains usually between 10 and 16 carbons in length. Two additional acyl chains are often attached to the 3-hydroxy groups of the primary acyl chains. E. coli lipid A, as an example, typically has four C14 3-hydroxy acyl chains attached to the sugars and one C12 and one C14 attached to the 3-hydroxy groups of the primary acyl chains at the 2′ and 3′ position, respectively.

As used herein, the term “lipid A analog or derivative” refers to a molecule that resembles the structure and immunological activity of lipid A, but that does not necessarily naturally occur in nature. Lipid A analogs or derivatives can be modified to be shortened or condensed, and/or to have their glucosamine residues substituted with another amine sugar residue, e.g. galactosamine residues, to contain a 2-deoxy-2-aminogluconate in place of the glucosamine-1-phosphate at the reducing end, to bear a galacturonic acid moiety instead of a phosphate at position 4′. Lipid A analogs or derivatives can be prepared from lipid A isolated from a bacterium, e.g., by chemical derivation, or chemically synthesized, e.g. by first determining the structure of the preferred lipid A and synthesizing analogs or derivatives thereof. Lipid A analogs or derivatives are also useful as TLR4 agonist adjuvants (see, e.g. Gregg K A et al, MBio 8, eDD492-17, doi: 10.1128/mBio.00492-17 (2017), which is hereby incorporated by reference in its entirety).

MPL and 3D-MPL are lipid A analogs or derivatives that have been modified to attenuate lipid A toxicity. Lipid A, MPL, and 3D-MPL have a sugar backbone onto which long fatty acid chains are attached, wherein the backbone contains two 6-carbon sugars in glycosidic linkage, and a phosphoryl moiety at the 4 position. Typically, five to eight long chain fatty acids (usually 12-14 carbon atoms) are attached to the sugar backbone. Due to derivation of natural sources, MPL or 3D-MPL can be present as a composite or mixture of a number of fatty acid substitution patterns, e.g. hepta-acyl, hexa-acyl, penta-acyl, etc., with varying fatty acid lengths. This is also true for some of the other lipid A analogs or derivatives described herein, however synthetic lipid A variants can also be defined and homogeneous. MPL and its manufacture are described in U.S. Pat. No. 4,436,727, which is hereby incorporated by reference in its entirety. 3D-MPL is described in U.S. Pat. No. 4,912,094B1 (which is hereby incorporated by reference in its entirety), and differs from MPL by selective removal of the 3-hydroxymyristic acyl residue that is ester linked to the reducing-end glucosamine at position 3. Examples of lipid A (analogs, derivatives) suitable for inclusion in the vaccine compositions described herein include MPL, 3D-MPL, RC529 (see e.g., EP1385541, which is hereby incorporated by reference in its entirety), PET-lipid A, GLA (glycopyranosyl lipid adjuvant, a synthetic disaccharide glycolipid; see e.g. US20100310602 and U.S. Pat. No. 8,722,064, which are hereby incorporated by reference in their entirety), SLA (see e.g. Carter D et al, Clin. Transl. Immunology 5: e108 (doi:10.1038/cti.2016.63) (2016), which is hereby incorporated by reference in its entirety and which describes a structure-function approach to optimize TLR4 ligands for human vaccines), PHAD (phosphorylated hexaacyl disaccharide; the structure of which is the same as that of GLA), 3D-PHAD, 3D-(6-acyl)-PHAD (3D(6A)-PHAD), E6020 (CAS Number 287180-63-6), ONO4007, OM-174, and the like. In any embodiment, the adjuvant of the vaccine composition is a TLR4 agonist adjuvant comprising a lipid A analog or derivative chosen from 3D-MPL, GLA, or SLA. In certain embodiments the lipid A analog or derivative is formulated in liposomes.

The adjuvant, preferably including a TLR4 agonist, may be formulated in various ways, e.g. in an emulsion, such as a water-in-oil (w/o) emulsion or an oil-in-water (o/w) emulsion (examples are MF59, AS03), stable (nano-)emulsions (SE), lipid suspensions, liposomes, (polymeric) nanoparticles, virosomes, alum adsorbed, aqueous formulations (AF), and the like, representing various delivery systems for immunomodulatory molecules in the adjuvant and/or for the immunogens (see e.g. Reed et al, Nature Med, 19: 1597-1608 (2013) and Alving C R et al, Curr Opin Immunol 24: 310-315 (2012), which are hereby incorporated by reference in their entirety).

In any embodiment, the immunostimulatory TLR4 agonist may optionally be combined with other immunomodulatory components, such as squalene oil-in-water emulsion (SE) (e.g., MF59; AS03); saponins (e.g. QuilA, QS7, QS21, Matrix M, Iscoms, Iscomatrix, etc); aluminum salts; activators for other TLRs (e.g. imidazoquinolines, flagellin, dsRNA analogs, TLR9 agonists, such as CpG, etc); and the like (see e.g. Reed G. et al., Nature Med, 19: 1597-1608 (2013), which is hereby incorporated by reference in its entirety).

In any embodiment, the adjuvant of the vaccine composition disclosed herein is a combination of a TLR4 agonist, e.g., GLA, in combination with SE (i.e., GLA-SE). In any embodiment, the adjuvant of the vaccine composition disclosed herein in a combination of a TLR4 agonist e.g., GLA, in combination with a saponin (e.g., GLS-QS21). In any embodiment, the aforementioned adjuvants can be formulated as liposomes. An exemplary adjuvant thus also includes GLA-LSQ, which comprises a synthetic TLR4 agonist (e.g., MPL [GLA]) and a saponin (e.g., QS21), formulated as liposomes.

Additional exemplary adjuvants for use in the vaccine compositions described herein comprise a lipid A analog or derivative and include, for example, SLA-SE (synthetic MPL [SLA], squalene oil/water emulsion), SLA-Nanoalum (synthetic MPL [SLA], aluminum salt), GLA-Nanoalum (synthetic MPL [GLA], aluminum salt), SLA-AF (synthetic MPL [SLA], aqueous suspension), GLA-AF (synthetic MPL [GLA], aqueous suspension,), SLA-alum (synthetic MPL [SLA], aluminum salt), GLA-alum (synthetic MPL [GLA], aluminum salt), AS01 (MPL, QS21, liposomes), AS02 (MPL, QS21, oil/water emulsion), AS25 (MPL, oil/water emulsion), AS04 (MPL, aluminum salt), and AS15 (MPL, QS21, CpG, liposomes). See, e.g., WO2008/153541; WO2010/141861; WO2013/119856; WO2019/051149; WO 2013/119856; WO 2006/116423; U.S. Pat. Nos. 4,987,237; 4,436,727; 4,877,611; 4,866,034; 4,912,094; 4,987,237; 5,191,072; 5,593,969; 6,759,241; 9,017,698; 9,149,521; 9,149,522; 9,415,097; 9,415,101; 9,504,743; Reed G. et al., Nature Med, 19: 1597-1608 (2013), Johnson et al., J Med Chem, 42:4640-4649 (1999), and Ulrich and Myers, 1995, Vaccine Design: The Subunit and Adjuvant Approach; Powell and Newman, Eds.; Plenum: New York, 495-524, which are hereby incorporated by reference in their entirety.

In any embodiment, the LukA and/or LukB proteins or polypeptides thereof of the vaccine composition may be conjugated to an immunogenic carrier molecule. Suitable immunogenic carrier molecules include, without limitation, bovine serum albumin, chicken egg ovalbumin, keyhole limpet hemocyanin, tetanus toxoid, diphtheria toxoid, thyroglobulin, a pneumococcal capsular polysaccharide, CRM 197, and a meningococcal outer membrane protein.

The vaccine composition may further include one or more additional S. aureus antigens selected from the group consisting of a serotype 336 polysaccharide antigen, clumping factor A, clumping factor B, a fibrinogen binding protein, a collagen binding protein, an elastin binding protein, a MHC analogous protein, a polysaccharide intracellular adhesion, beta hemolysin, delta hemolysin, Panton-Valentine leukocidin, leukocidin M, exfoliative toxin A, exfoliative toxin B, V8 protease, hyaluronate lyase, lipase, staphylokinase, an enterotoxin, an enterotoxin superantigen SEA, an enterotoxin superantigen SAB, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, Sbi, Type 5 antigen, Type 8 antigen, and lipoteichoic acid. Other S. aureus antigen suitable for inclusion in the vaccine compositions described herein include, without limitation, CP5, CP8, Eap, Ebh, Emp, EsaB, EsaC, EsxA, EsxB, EsxAB(fusion), IsdA, IsdB, IsdC, MntC, rTSST-1, rTSST-1v, TSST-1, SasF, vWbp, vWh vitronectin binding protein, Aaa, Aap, Ant, autolysin glucosaminidase, autolysin amidase, Can, collagen binding protein, CsalA, EFB, Elastin binding protein, EPB, FbpA, fibrinogen binding protein, Fibronectin binding protein, FhuD, FhuD2, FnbA, FnbB, GehD, HarA, HBP, Immunodominant ABC transporter, IsaA/PisA, laminin receptor, Lipase GehD, MAP, Mg2+transporter, MHC II analog, MRPII, NPase, RNA III activating protein (RAP), SasA, SasB, SasC, SasD, SasK, SBI, SEA exotoxins, SEB exotoxins, mSEB, SitC, Ni ABC transporter, SitC/MntC/saliva binding protein, SsaA, SSP-1, SSP-2, Sta006, and Sta011.

In any embodiment, the vaccine composition is formulated as an injectable which can be injected, for example, via an injection device (e.g., a syringe or an infusion pump). The injection can be delivered intramuscularly, intraperitoneally, intravitreally, or intravenously, for example.

The vaccine composition of the present disclosure may be formulated for parenteral administration. Solutions, suspensions, or emulsions of the composition can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical vaccine compositions suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

In any embodiment, the vaccine composition as described herein is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The vaccine composition can also be in the form of sachets, dragees, powders, granules, lozenges, or powders for reconstitution, for example.

The dosage forms of the vaccine composition may be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the vaccine composition can be delivered intranasally, intrabuccally, or sublingually.

The pH in an aqueous formulation of the vaccine composition can be between pH 3 and pH 10. In one embodiment, the pH of the vaccine composition is from about 7.0 to about 9.5. In another embodiment, the pH of the vaccine composition is from about 3.0 to about 7.0.

Use of the S. aureus Vaccine Compositions

Another aspect of the present disclosure relates to use of the vaccine compositions described herein to prevent or inhibit the onset of S. aureus infection in a subject. In one embodiment, the disclosure is directed to a method of generating an immune response against S. aureus in a subject, that involves administering the vaccine composition as described herein to a subject under conditions effective to generate said immune response against S. aureus in said subject. Another embodiment is directed to a method of treating or preventing a S. aureus infection in a subject in need thereof, that involves administering an effective amount of the vaccine composition as disclosed herein. Another embodiment is directed to a method for decolonization or preventing colonization or recolonization of S. aureus in a subject in need thereof that involves administering an effective amount of the vaccine composition as disclosed herein. In accordance with this aspect, the methods described herein are suitable for preventing short term and persistent colonization or recolonization of S. aureus in a subject in need thereof.

These methods involve administering any one of the vaccine compositions described herein to a subject in need thereof, e.g., a subject at risk of S. aureus exposure or infection. In one embodiment, the vaccine composition comprises a LukA variant polypeptide (i.e., a variant of SEQ ID NOs: 1 or 2) and a wild-type LukB protein or polypeptide as described supra. In another embodiment, the vaccine composition comprises a wild-type LukA protein or polypeptide and a LukB variant polypeptide (i.e., a variant of SEQ ID NOs: 15 or 16) as described supra. In another embodiment, the vaccine composition comprises a LukA variant polypeptide and a LukB variant polypeptide as described supra. A suitable subject for treatment in accordance with this aspect of the present disclosure is a subject at risk of developing a S. aureus infection.

In accordance with this aspect of the present disclosure, a prophylactically effective amount of the vaccine composition is administered to the subject to generate an immune response against S. aureus infection. A prophylactically effective amount is the amount necessary to generate or elicit a humoral (i.e., antibody mediated) and cellular (T-cells) immune responses. The elicited humoral response is sufficient to prevent or at least reduce the extent of S. aureus infection that would otherwise develop in the absence of such response. Preferably, administration of a prophylactically effective amount of the vaccine composition described herein induces a neutralizing immune response against S. aureus in the subject. To effectuate an effective immune response in a subject, the composition may further contain one or more additional S. aureus antigens or an adjuvant as described supra. In an alternative embodiment, the adjuvant is administered separately from the composition to the subject, either before, after, or concurrent with administration of the composition of the present disclosure.

For purposes of this aspect of the disclosure, the target “subject” encompasses any animal, preferably a mammal, more preferably a human. In the context of administering a vaccine composition for purposes of preventing, inhibiting, or reducing the severity of a S. aureus infection and S. aureus colonization in a subject, the target subject encompasses any subject that is at risk of being infected by S. aureus. Particularly susceptible subjects include immunocompromised infants, juveniles, adults, and elderly adults. However, any infant, juvenile, adult, or elderly adult at risk for S. aureus infection can be treated in accordance with the methods and vaccine composition described herein. Particularly suitable subjects include those at risk of infection with methicillin-resistant S. aureus (MRSA) or methicillin sensitive S. aureus (MSSA). Other suitable subjects include those subjects which may have or are at risk for developing a condition resulting from a S. aureus infection, i.e., a S. aureus associated condition, such as, for example, skin wounds and infections, tissue abscesses, folliculitis, osteomyelitis, pneumonia, scalded skin syndrome, septicemia, septic arthritis, myocarditis, endocarditis, and toxic shock syndrome.

In any embodiment, the subject is at least or at most 1, 2, 3, 4, 5, 6, 7, 8, 9, 10, 11, 12, 13, 14, 15, 16, 17, 18, 19, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 85, or 90 years old (or any range derivable therein). In certain embodiments, the subject or patient described herein, such as the human subject, is a pediatric subject. A pediatric subject is one that is defined as less than 18 years old. In any embodiment, the pediatric subject t is 2 years old or less. In any embodiment, the pediatric subject is less than 1-year-old. In any embodiment, the pediatric subject is less than 6 months old. In any embodiment, the pediatric subject is 2 months old or less. In any embodiment, the human patient is 65 years old or older. In any embodiment, the human patient is a health care worker. In any embodiment, the patient is one that will receive a surgical procedure.

Numerous other factors may also be accounted for when administering the vaccine composition under conditions effective to induce a robust immune response. These factors include, for example and without limitation, the concentration of the active agents in the composition, the mode and frequency of administration, and the subject details, such as age, weight and overall health and immune condition. General guidance can be found, for example, in the publications of the International Conference on Harmonization and in REMINGTON'S PHARMACEUTICAL SCIENCES (Mack Publishing Company 1990), which is hereby incorporated by reference in its entirety. A clinician may administer a vaccine composition as described herein until a dosage is reached that provides the desired or required prophylactic effect, e.g., the desired antibody titers. The progress of the prophylactic response can be easily monitored by conventional assays.

In one embodiment of the present disclosure, the vaccine composition as described herein is administered prophylactically to prevent, delay, or inhibit the development of S. aureus infection in a subject at risk of being infected with S. aureus or at risk of developing an associated condition. In any embodiment of the present disclosure, prophylactic administration of the vaccine composition is effective to fully prevent S. aureus infection in an individual. In other embodiments, prophylactic administration is effective to prevent the full extent of infection that would otherwise develop in the absence of such administration, i.e., substantially prevent or inhibit S. aureus infection in an individual.

In the context of using prophylactic compositions to prevent S. aureus infection, the dosage of the composition is one that is adequate to generate an antibody titer capable of neutralizing S. aureus LukAB mediated cytotoxicity and is capable of achieving a reduction in a number of symptoms, a decrease in the severity of at least one symptom, or a delay in the further progression of at least one symptom, or even a total alleviation of the infection.

Prophylactically effective amounts of the vaccine compositions described herein will depend on whether an adjuvant is co-administered, with higher dosages being required in the absence of adjuvant. The amount of variant LukA and/or LukB for administration can vary from 1 μg-500 μg per patient. In any embodiment, 5, 10, 20, 25, 50 or 100 μg is used for each human injection. Occasionally, a higher dose of 1-50 mg per injection is used. Typically, about 10, 20, 30, 40, or 50 mg is used for each human injection. The timing of injections can vary significantly from once a year to once a decade. Generally, an effective dosage can be monitored by obtaining a fluid sample from the subject, generally a blood serum sample, and determining the titer of antibody developed against LukA, LukB or LukAB, using methods well known in the art and readily adaptable to the specific antigen to be measured. Ideally, a sample is taken prior to initial dosing and subsequent samples are taken and titered after each immunization. Generally, a dose or dosing schedule which provides a detectable titer at least four times greater than control or “background” levels at a serum dilution of 1:100 is desirable, where background is defined relative to a control serum or relative to a plate background in ELISA assays.

The vaccine compositions of the present disclosure can be administered by parenteral, topical, intravenous, oral, intraperitoneal, intranasal or intramuscular means for prophylactic treatment.

The vaccine compositions of the present disclosure may be formulated for parenteral administration. Solutions, suspensions, or emulsions of the composition can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical vaccine formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

### EMBODIMENTS

The disclosure provides the following non-limiting embodiments.

Embodiment 1 is a variant Staphylococcus aureus Leukocidin A (LukA) polypeptide of SEQ ID NO:25, said LukA variant polypeptide comprising: an amino acid substitution at one or more amino acid residues corresponding to amino acid residues Lys83, Ser141, Val113, and Val193 of SEQ ID NO: 25.

Embodiment 2 is the LukA variant polypeptide of embodiment 1, wherein said LukA variant polypeptide further comprises an amino acid substitution at the amino acid residue corresponding to Glu323 of SEQ ID NO: 25.

Embodiment 3 is the LukA variant polypeptide of embodiment 2, wherein the amino acid substitution at the amino acid residue corresponding to Glu323 comprises a glutamic acid to alanine (Glu323Ala) substitution.

Embodiment 4 is the LukA variant polypeptide of any one of embodiments 1-3, wherein the amino acid substitution at the amino acid residue corresponding to Lys83 comprises a lysine to methionine (Lys83Met) substitution.

Embodiment 5 is the LukA variant polypeptide of any one of embodiments 1-4, wherein the amino acid substitution at the amino acid residue corresponding to Ser141 comprises a serine to alanine (Ser141Ala) substitution.

Embodiment 6 is the LukA variant polypeptide of any one of embodiments 1-5, wherein the amino acid substitution at the amino acid residue corresponding to Val113 comprises a valine to isoleucine (Val113Ile) substitution.

Embodiment 7 is the LukA variant polypeptide of any one of embodiments 1-6, wherein the amino acid substitution at the amino acid residue corresponding to Val193 comprises a valine to isoleucine (Val193Ile) substitution.

Embodiment 8 is the LukA variant polypeptide of any one of embodiments 2-7, wherein said LukA variant polypeptide comprises amino acid substitutions at each amino acid residue corresponding to amino acid residues Lys83, Ser141, Val113, Val193, and Glu323 of SEQ ID NO: 25.

Embodiment 9 is the LukA variant polypeptide of embodiment 8, wherein the amino acid substitutions comprise Lys83Met, Ser141Ala, Val113Ile, Val193Ile, and Glu323Ala.

Embodiment 10 is the LukA variant polypeptide of embodiment 1, wherein said variant is a CC8 LukA variant of SEQ ID NO: 1 comprising amino acid substitutions corresponding to Lys80Met, Ser138Ala, Val110Ile, Val190Ile, and Glu320Ala in SEQ ID NO: 1.

Embodiment 11 is the LukA variant polypeptide of embodiment 10, wherein said LukA variant polypeptide comprises the amino acid sequence of SEQ ID NO: 3.

Embodiment 12 is the LukA variant polypeptide of embodiment 1, wherein said variant is a CC45 LukA variant of SEQ ID NO: 2 comprising amino acid substitutions corresponding to Lys81Met, Ser139Ala, Val111Ile, Val191Ile, and Glu321Ala in SEQ ID NO: 2.

Embodiment 13 is the LukA variant polypeptide of embodiment 12, wherein said LukA variant polypeptide comprises the amino acid sequence of SEQ ID NO: 4.

Embodiment 14 is the LukA variant polypeptide of any one of embodiments 1-13, wherein said LukA variant polypeptide further comprises an amino acid substitution at one or more amino acid residues corresponding to amino acid residues Tyr74, Asp140, Gly149, and Gly156 of SEQ ID NO: 25.

Embodiment 15 is the LukA variant polypeptide of embodiment 14, wherein the amino acid substitution at the amino acid residue corresponding to Tyr74 comprises a tyrosine to cysteine (Tyr74Cys) substitution.

Embodiment 16 is the LukA variant polypeptide of embodiment 14 or embodiment 15, wherein the amino acid substitution at the amino acid residue corresponding to Asp140 comprises an asparagine to cysteine (Asp140Cys) substitution.

Embodiment 17 is the LukA variant polypeptide of any one of embodiments 14-16, wherein the amino acid substitution at the amino acid residue corresponding to Gly149 comprises a glycine to cysteine (Gly149Cys) substitution.

Embodiment 18 is the LukA variant polypeptide of any one of embodiments 14-17, wherein the amino acid substitution at the amino acid residue corresponding to Gly156 comprises a glycine to cysteine (Gly156Cys) substitution.

Embodiment 19 is the LukA variant polypeptide of any one of embodiments 14-18, wherein said LukA variant polypeptide comprises amino acid substitutions at each amino acid residue corresponding to amino acid residues Tyr74, Asp140, Gly149, and Gly156 of SEQ ID NO: 25

Embodiment 20 is the LukA variant polypeptide of embodiment 14, wherein said variant is a CC8 LukA variant of SEQ ID NO: 1 comprising amino acid substitutions corresponding to Lys80Met, Ser138Ala, Val10Ile, Val190Ile, Glu320Ala, Tyr71Cys, Asp137Cys, Gly146Cys, and Gly153Cys of SEQ ID NO: 1.

Embodiment 21 is the LukA variant polypeptide of embodiment 20, wherein said LukA variant polypeptide comprises the amino acid sequence of SEQ ID NO: 5.

Embodiment 22 is the LukA variant polypeptide of embodiment 14, wherein said variant is a CC45 LukA variant of SEQ ID NO: 2 comprising amino acid substitutions corresponding to Lys81Met, Ser139Ala, Val111Ile, Val191Ile, Glu321Ala, Tyr72Cys, Asp138Cys, Gly147Cys, and Gly154Cys of SEQ ID NO: 2.

Embodiment 23 is the LukA variant polypeptide of embodiment 22, wherein said LukA variant polypeptide comprises the amino acid sequence of SEQ ID NO: 6.

Embodiment 24 is the LukA variant polypeptide of any one of embodiments 1-23, wherein said LukA variant polypeptide further comprises an amino acid substitution at the amino acid residue corresponding to amino acid residue Thr249 of SEQ ID NO: 25.

Embodiment 25 is the LukA variant polypeptide of embodiment 24, wherein the amino acid substitution at the amino acid residue corresponding to Thr249 comprises a threonine to valine (Thr249Val) substitution.

Embodiment 26 is the LukA variant polypeptide of embodiment 25, wherein said variant LukA protein comprises the amino acid sequence of SEQ ID NO: 7 or SEQ ID NO: 8

Embodiment 27 is the LukA variant polypeptide of any one of embodiments 1-26 further comprising an amino-terminus signal sequence.

Embodiment 28 is the LukA variant polypeptide of embodiment 27, wherein the amino-terminus signal sequence comprises the amino acid sequence of SEQ ID NO: 23.

Embodiment 29 is the LukA variant polypeptide of any one of embodiments 1-28 further comprising an amino-terminus purification sequence.

Embodiment 30 is a nucleic acid molecule encoding the LukA variant polypeptide of any one of embodiments 1-29.

Embodiment 31 is an expression vector comprising the nucleic acid molecule of embodiment 30.

Embodiment 32 is a host cell comprising the expression vector of embodiment 31.

Embodiment 33 is a variant Staphylococcus aureus Leukocidin B (LukB) protein or polypeptide of SEQ ID NO: 39, said LukB variant polypeptide comprising an amino acid substitution at the amino acid residue corresponding to amino acid residue Val53 of SEQ ID NO: 39.

Embodiment 34 is the LukB variant polypeptide of embodiment 33, wherein the amino acid substitution at the amino acid residue corresponding to Val53 comprises a valine to leucine (Val53Leu) substitution.

Embodiment 35 is the LukB variant polypeptide of embodiment 33, wherein said variant is a CC8 LukB variant of SEQ ID NO: 15 comprising the amino acid substitution corresponding to Val53Leu of SEQ ID NO: 15.

Embodiment 36 is the LukB variant polypeptide of embodiment 35, wherein said LukB variant polypeptide comprises the amino acid sequence of SEQ ID NO: 17.

Embodiment 37 is the LukB variant polypeptide of embodiment 33, wherein said variant is a CC45 LukB variant of SEQ ID NO: 16 comprising the amino acid substitution corresponding to Val53Leu of SEQ ID NO: 16.

Embodiment 38 is the LukB variant polypeptide of embodiment 37, wherein said LukB variant polypeptide comprises the amino acid sequence of SEQ ID NO: 18.

Embodiment 39 is the LukB variant polypeptide of embodiment 33 or embodiment 34, wherein said variant further comprises an amino acid substitution at one or more amino acid residues corresponding to amino acid residues Glu45, Glu109, Thr121, and Arg154 of SEQ ID NO: 39.

Embodiment 40 is the LukB variant polypeptide of embodiment 39, wherein the amino acid substitution at the amino acid residue corresponding to Glu45 comprises a glutamic acid to cysteine (Glu45Cys) substitution.

Embodiment 41 is the LukB variant polypeptide of embodiment 39 or embodiment 40, wherein the amino acid substitution at the amino acid residue corresponding to Glu109 comprises a glutamic acid to cysteine (Glu109Cys) substitution.

Embodiment 42 is the LukB variant polypeptide of any one of embodiments 39-41, wherein the amino acid substitution at the amino acid residue corresponding to Thr121 comprises a threonine to cysteine (Thr121Cys) substitution.

Embodiment 43 is the LukB variant polypeptide of any one of embodiments 39-42, wherein the amino acid substitution at the amino acid residue corresponding to Arg154 comprises an arginine to cysteine (Arg154Cys) substitution.

Embodiment 44 is the LukB variant polypeptide of any one of embodiments 39-43, wherein said LukB variant polypeptide comprises amino acid substitutions at each amino acid residue corresponding to amino acid residues Glu45, Glu109, Thr121, and Arg154 of SEQ ID NO: 39.

Embodiment 45 is the LukB variant polypeptide of embodiment 39, wherein said variant is a CC8 LukB variant of SEQ ID NO: 15 comprising the amino acid substitutions corresponding to Val53Leu, Glu45Cys, Glu109Cys, Thr121Cys, and Arg154Cys of SEQ ID NO: 15.

Embodiment 46 is the LukB variant polypeptide of embodiment 45, wherein said LukB variant polypeptide comprises the amino acid sequence of SEQ ID NO: 19.

Embodiment 47 is the LukB variant polypeptide of embodiment 39, wherein said variant is a CC45 LukB variant of SEQ ID NO: 16 comprising the amino acid substitutions corresponding to Val53Leu, Glu45Cys, Glu110Cys, Thr123Cys, and Arg155Cys of SEQ ID NO: 16.

Embodiment 48 is the LukB variant polypeptide of embodiment 47, wherein said LukB variant polypeptide comprises the amino acid sequence of SEQ ID NO: 20.

Embodiment 49 is a variant Staphylococcus aureus Leukocidin B (LukB) protein or polypeptide of SEQ ID NO:39, said LukB variant polypeptide comprising: an amino acid substitution at one or more amino acid residues corresponding to amino acid residues Glu45, Glu109, Thr121, and Arg154 of SEQ ID NO: 39.

Embodiment 50 is the LukB variant polypeptide of embodiment 49, wherein the amino acid substitution at the amino acid residue corresponding to Glu45 comprises a glutamic acid to cysteine (Glu45Cys) substitution and the amino acid substitution at the amino acid residue corresponding to Thr121 comprises a threonine to cysteine (Thr121Cys) substitution.

Embodiment 51 is the LukB variant polypeptide of embodiment 49 or embodiment 50, wherein the amino acid substitution at the amino acid residue corresponding to Glu109 comprises a glutamic acid to cysteine (Glu109Cys) substitution and the amino acid substitution at the amino acid residue corresponding to Arg154 comprises an arginine to cysteine (Arg154Cys) substitution.

Embodiment 52 is the LukB variant polypeptide of any one of embodiments 49-51, wherein said LukB variant polypeptide comprises amino acid substitutions at each amino acid residue corresponding to amino acid Glu45, Glu109, Thr121, and Arg154 of SEQ ID NO: 39.

Embodiment 53 is the LukB variant polypeptide of embodiment 52, wherein said variant is a CC8 LukB variant of SEQ ID NO: 15 comprising the amino acid substitutions corresponding to Glu45Cys, Glu109Cys, Thr121Cys, and Arg154Cys of SEQ ID NO: 15.

Embodiment 54 is the LukB variant polypeptide of embodiment 53, wherein said LukB variant polypeptide comprises the amino acid sequence of SEQ ID NO: 21.

Embodiment 55 is the LukB variant polypeptide of embodiment 52, wherein said variant is a CC45 LukB variant of SEQ ID NO: 16 comprising the amino acid substitutions corresponding to Glu45Cys, Glu110Cys, Thr123Cys, and Arg155Cys of SEQ ID NO: 16.

Embodiment 56 is the LukB variant polypeptide of embodiment 55, wherein said LukB variant polypeptide comprises the amino acid sequence of SEQ ID NO: 22.

Embodiment 57 is the LukB variant polypeptide of any one of embodiments 33-56 further comprising: an amino-terminus signal sequence.

Embodiment 58 is the LukB variant polypeptide of embodiment 57, wherein the amino-terminus signal sequence comprises the amino acid sequence of SEQ ID NO: 23

Embodiment 59 is the LukB variant polypeptide of any one of embodiments 33-58 further comprising: an amino-terminus purification tag.

Embodiment 60 is a nucleic acid molecule encoding the LukB variant polypeptide of any one of embodiments 33-59.

Embodiment 61 is an expression vector comprising the nucleic acid molecule of embodiment 60.

Embodiment 62 is an expression vector comprising the nucleic acid molecule of embodiment 30 operably coupled to the nucleic acid molecule of embodiment 60.

Embodiment 63 is the expression vector of embodiment 62 comprising a nucleotide sequence having at least 85%, at least 90%, at least 95%, at least 97%, or at least 99% sequence similarity to the nucleotide sequence of SEQ ID NO: 55 (CC45 LukA variant) operatively coupled to a nucleotide sequence having at least 85%, at least 90%, at least 95%, at least 97%, or at least 99% sequence similarity to the nucleotide sequence of SEQ ID NO: 59 (CC45 LukB).

Embodiment 64 is an expression vector of embodiment 62 comprising a nucleotide sequence having at least 85%, at least 90%, at least 95%, at least 97%, or at least 99% sequence similarity to the nucleotide sequence of SEQ ID NO: 55 (CC45 LukA variant) operatively coupled to a nucleotide sequence having at least 85%, at least 90%, at least 95%, at least 97%, or at least 99% sequence similarity to the nucleotide sequence of SEQ ID NO: 61 (CC45 LukB variant).

Embodiment 65 is an expression vector of embodiment 62 comprising a nucleotide sequence having at least 85%, at least 90%, at least 95%, at least 97%, or at least 99% sequence similarity to the nucleotide sequence of SEQ ID NO: 54 (CC8 LukA variant) operatively coupled to a nucleotide sequence having at least 85%, at least 90%, at least 95%, at least 97%, or at least 99% sequence similarity to the nucleotide sequence of SEQ ID NO: 58 (CC8 LukB).

Embodiment 66 is an expression vector of embodiment 62 comprising a nucleotide sequence having at least 85%, at least 90%, at least 95%, at least 97%, or at least 99% sequence similarity to the nucleotide sequence of SEQ ID NO: 54 (CC8 LukA variant) operatively coupled to a nucleotide sequence having at least 85%, at least 90%, at least 95%, at least 97%, or at least 99% sequence similarity to the nucleotide sequence of SEQ ID NO: 61 (CC45 LukB variant).

Embodiment 67 is an expression vector of embodiment 62 comprising a nucleotide sequence having at least 85%, at least 90%, at least 95%, at least 97%, or at least 99% sequence similarity to the nucleotide sequence of SEQ ID NO: 54 (CC8 LukA variant) operatively coupled to a nucleotide sequence having at least 85%, at least 90%, at least 95%, at least 97%, or at least 99% sequence similarity to the nucleotide sequence of SEQ ID NO: 60 (CC8 LukB variant).

Embodiment 68 is a host cell comprising the expression vector of any one of embodiments 61-67.

Embodiment 69 is a Staphylococcus aureus vaccine composition comprising the expression vector of any one of embodiments 61-67

Embodiment 70 is a Staphylococcus aureus vaccine composition comprising one or more variant LukA variant polypeptides of any one of embodiments 1-29.

Embodiment 71 is the vaccine composition of embodiment 70, wherein the LukA variant polypeptide is a variant of SEQ ID NO: 1.

Embodiment 72 is the vaccine composition of embodiment 70 or embodiment 71 further comprising: a leukocidin B (LukB) protein or polypeptide, said LukB protein or polypeptide having at least 85% sequence similarity to the amino acid sequence of SEQ ID NO: 15.

Embodiment 73 is the vaccine composition of embodiment 70 or embodiment 71 further comprising a leukocidin B (LukB) protein or polypeptide, said LukB protein or polypeptide having at least 85% sequence similarity to the amino acid sequence of SEQ ID NO: 16.

Embodiment 74 is the vaccine composition of embodiment 73, wherein the LukA variant polypeptide comprises an amino acid substitution at one or more amino acid residues corresponding to amino acid residues Lys80, Ser138, Val110, Val190, and Glu320Ala of SEQ ID NO: 1.

Embodiment 75 is the vaccine composition of embodiment 74, wherein the LukA variant polypeptide comprises an amino acid sequence having at least 90% sequence identity to the amino acid sequence of SEQ ID NO: 3 and the LukB polypeptide comprises an amino acid sequence having at least 90% sequence identity to the amino acid sequence of SEQ ID NO:18.

Embodiment 76 is the vaccine composition of embodiment 74, wherein the LukA variant polypeptide comprises the amino acid sequence SEQ ID NO: 3 and the LukB polypeptide comprises the amino acid sequence of SEQ ID NO:18.

Embodiment 77 is the vaccine composition of embodiment 74, wherein the LukA variant polypeptide comprises the amino acid sequence of SEQ ID NO: 3 and the LukB protein or polypeptide comprises the amino acid sequence of SEQ ID NO: 22.

Embodiment 78 is the vaccine composition of embodiment 74, wherein the LukA variant polypeptide comprises the amino acid sequence of SEQ ID NO: 3 and the LukB protein or polypeptide comprises the amino acid sequence of SEQ ID NO: 20.

Embodiment 79 is the vaccine composition of embodiment 70, wherein the LukA variant polypeptide is a variant of SEQ ID NO: 2.

Embodiment 80 is the vaccine composition of embodiment 79 further comprising a leukocidin B (LukB) protein or polypeptide, said LukB protein or polypeptide having at least 85% sequence similarity to the amino acid sequence of SEQ ID NO: 16.

Embodiment 81 is the vaccine composition of embodiment 79 further comprising a leukocidin B (LukB) protein or polypeptide, said LukB protein or polypeptide having at least 85% sequence similarity to the amino acid sequence of SEQ ID NO: 15.

Embodiment 82 is the vaccine composition of any one of embodiments 79-81, wherein the LukA variant polypeptide comprises an amino acid substitution at one or more amino acid residues corresponding to amino acid residues Lys81, Ser139, Val111, Val191, and Glu321Ala of SEQ ID NO: 2.

Embodiment 83 is the vaccine composition of embodiment 82, wherein the LukA variant polypeptide comprises an amino acid sequence having at least 90% sequence identity to the amino acid sequence of SEQ ID NO: 4 and the LukB protein or polypeptide comprises an amino acid sequence having at least 90% sequence identity to the amino acid sequence of SEQ ID NO:16

Embodiment 84 is the vaccine composition of embodiment 82, wherein the LukA variant polypeptide comprises the amino acid sequence SEQ ID NO: 4 and the LukB protein or polypeptide comprises the amino acid sequence of SEQ ID NO:16.

Embodiment 85 is a Staphylococcus aureus vaccine composition comprising one or more variant LukB proteins or polypeptides of any one of embodiments 33-56.

Embodiment 86 is the vaccine composition of embodiment 85 further comprising a leukocidin A (LukA) protein or polypeptide, said LukA protein or polypeptide having at least 85% sequence similarity to the amino acid sequence of SEQ ID NO: 1 (CC8).

Embodiment 87 is the vaccine composition of embodiment 85 further comprising a leukocidin A (LukA) protein or polypeptide, said LukA protein or polypeptide having at least 85% sequence similarity to the amino acid sequence of SEQ ID NO: 2 (CC45).

Embodiment 88 is a Staphylococcus aureus vaccine composition comprising the LukA variant polypeptide of any one of embodiments 1-32, and the LukB variant polypeptide of any one of embodiments 33-56.

Embodiment 89 is the vaccine composition of embodiment 74, wherein the LukA variant polypeptide comprises an amino acid sequence having at least 90% sequence identity to the amino acid sequence of SEQ ID NO: 3 and the LukB protein or polypeptide comprises an amino acid sequence having at least 90% sequence identity to the amino acid sequence of SEQ ID NO:15.

Embodiment 90 is the vaccine composition of embodiment 74, wherein the LukA variant polypeptide comprises the amino acid sequence of SEQ ID NO: 3 and the LukB protein or polypeptide comprises the amino acid sequence of SEQ ID NO: 15.

Embodiment 91 is the vaccine composition of embodiment 74, wherein the LukA variant polypeptide comprises an amino acid sequence having at least 90% sequence identity to the amino acid sequence of SEQ ID NO: 3 and the LukB protein or polypeptide comprises an amino acid sequence having at least 90% sequence identity to the amino acid sequence of SEQ ID NO:17.

Embodiment 92 is the vaccine composition of embodiment 74, wherein the LukA variant polypeptide comprises the amino acid sequence of SEQ ID NO: 3 and the LukB protein or polypeptide comprises the amino acid sequence of SEQ ID NO: 17.

Embodiment 93 is the vaccine composition of embodiment 74, wherein the LukA variant polypeptide comprises an amino acid sequence having at least 90% sequence identity to the amino acid sequence of SEQ ID NO: 4 and the LukB protein or polypeptide comprises an amino acid sequence having at least 90% sequence identity to the amino acid sequence of SEQ ID NO:18.

Embodiment 94 is the vaccine composition of embodiment 74, wherein the LukA variant polypeptide comprises the amino acid sequence of SEQ ID NO: 4 and the LukB protein or polypeptide comprises the amino acid sequence of SEQ ID NO: 18

Embodiment 95 is the vaccine composition of any one of embodiments 64-69-94 further comprising an adjuvant.

Embodiment 96 is the vaccine composition of any one of embodiments 69-94 further comprising one or more additional S. aureus antigens.

Embodiment 97 is a method of generating an immune response against S. aureus in a subject, said method comprising: administering the vaccine composition of any one of embodiments 69-94 to the subject under conditions effective to generate said immune response against S. aureus in said subject.

Embodiment 98 is the vaccine composition of any one of embodiments 66-94 for use in a method of generating an immune response against S. aureus in a subject.

### Examples

The following examples are provided to illustrate embodiments of the present disclosure but are by no means intended to limit its scope.

### Example 1—Exemplary LukA Variant Polypeptides, LukB Variant Polypeptides, and Stable LukAB Complexes

For expression of LukAB heterodimeric proteins, E. coli BL21(DE3) cells were co-transformed with a lukA construct cloned into pCDFDuet-1 and a lukB construct cloned into pETDuet-1. Transformants were cultured in 50 pg/mL ampicillin and 50 pg/mL spectinomycin to select for pETDuet-1 and pCDFDuet-1, respectively, in Luria-Bertani broth at 37° C., with shaking at 190 rpm, overnight. For expression, fresh Terrific Broth media was inoculated with 1:50 dilution of the overnight culture at 37° C., with shaking at 190 rpm until cultures reached an OD₆₀₀=2. Expression was then induced through the addition of isopropyl β-d-1-thiogalactopyranoside to a final concentration of 1 mM, and induction continued at 37° C. for an additional 5 hours. The expression of LukAB heterodimers including pairs of cysteine substitutions in LukA and/or LukB were expressed in the cytoplasm of E. coli Origami 2(DE3) cells to support disulfide bond formation. The expression of LukA monomers in the periplasm of E. coli BL21(DE3) was performed through the transformation of lukA constructs in pD861-CH, with induction in Terrific Broth (supplemented with 30 pg/mL kanamycin) using a final concentration of 4 mM rhamnose at 37° C. for 4 hours. After induction of both cytoplasmic and periplasmic expression constructs, the cells were harvested through centrifugation at 4000 rpm at 4° C. for 15 min and then resuspended in lysis buffer (94% Bugbuster [EMD Millipore]+6% 5 M NaCl+0.4% 4 M imidazole+protease inhibitor cocktail [ProteaseArrest, G-Biosciences]). Following lysis at room temperature for 20 minutes, the lysates were incubated on ice for 45 minutes and then centrifuged at 16100×g, 4° C. for 35 minutes. Proteins were purified through the 6×His-tag at the N-terminus of LukA using an AKTA Pure 25M FPLC and HisTrap columns and eluted using an imidazole gradient (50-500 mM imidazole in 50 mM sodium phosphate buffer, pH 7.4, 200 mM NaCl). Fractions containing purified protein, as determined by SDS-PAGE, were pooled, and dialyzed in 50 mM sodium phosphate buffer, pH 7.4, 200 mM NaCl, 10% glycerol at 4° C. overnight. Purified proteins were quantified through the bicinchoninic acid (BCA) protein assay (Pierce).

TABLE 4
Exemplary LukA, LukB, and LukAB Heterodimer Complexes Utilized in the Studies Described Herein

| Toxoid/ | | | | | | |
|---|---|---|---|---|---|---|
| Toxin | | | SEQ | | LukB | SEQ ID |
| Name | LukA | LukA Substitutions | ID NO: | LukB | Substitutions | NO: |
| RARPR-013 | CC45 | E321A, Lys81Leu, Ser139Ala, | 11 | CC45 | none | 16 |
| | W94 | Val111Ile, Val191Ile | | | | |
| RARPR-015 | CC45 | E321A, Lys81Met, Ser139Ala, | 4 | CC45 | none | 16 |
| | W95 | Val111Ile, Val191Ile | | | | |
| RARPR-017 | CC45 | E321A, Lys81Leu, Ser139Ala, | 12 | CC45 | none | 16 |
| | W96 | Val111Ile, Val191Ile, Thr247Val | | | | |
| RARPR-019 | CC45 | E321A, Lys81Met, Ser139Ala, | 8 | CC45 | none | 16 |
| | W97 | Val111Ile, Val191Ile, Thr247Val | | | | |
| RARPR-30 | CC45 | E321A, Lys81Met, Ser139Ala, | 4 | CC45 | Val53Leu | 18 |
| | W95 | Val111Ile, Val191Ile | | | | |
| RARPR-31 | CC8 | E320A, Lys80Met, Ser138Ala, | 3 | none | | |
| | W95 | Val110Ile, Val190Ile | | | | |
| RARPR-32 | CC8 | E320A, Lys80Met, Ser138Ala, | 3 | CC8 | none | 15 |
| | W95 | Val110Ile, Val190Ile | | | | |
| RARPR-33 | CC8 | E320A, Lys80Met, Ser138Ala, | 3 | CC45 | Val53Leu | 18 |
| | W95 | Val110Ile, Val190Ile | | | | |
| RARPR-34 | CC8 | E320A, Lys80Met, Ser138Ala, | 3 | CC8 | Val53Leu | 17 |
| | W95 | Val110Ile, Val190Ile | | | | |
| LukA | CC8 | E320A, Lys80Met, Ser138Ala, | 9 | none | | |
| monomer | W97 | Val110Ile, Val190Ile, Thr246Val, | | | | |
| | W72 | Tyr71Cys, Asp137Cys, Gly146Cys, | | | | |
| | | Gly153Cys | | | | |
| LukA | CC45 | E321A, Lys81Met, Ser139Ala, Val111Ile, | 10 | none | | |
| monomer | W97 | Val191Ile, Thr247Val, Tyr72Cys, | | | | |
| | W72 | Asp138Cys, Gly147Cys, Gly154Cys | | | | |
| | CC8 | E320A, Lys80Met, Ser138Ala, | 5 | CC45 | none | 16 |
| | W95 | Val110Ile, Val190Ile, Tyr71Cys, | | | | |
| | W72 | Asp137Cys, Gly146Cys, Gly153Cys | | | | |
| | CC8 | E320A, Lys80Met, Ser138Ala, | 5 | CC45 | Glu45Cys, | 22 |
| | W95 | Val110Ile, Val190Ile, Tyr71Cys, | | | Thr122Cys, | |
| | W72 | Asp137Cys, Gly146Cys, Gly153Cys | | | Glu110Cys, | |
| | | | | | Arg155Cys | |
| | CC8 | E320A, Lys80Met, Ser138Ala, | 5 | CC45 | Val53Leu | 18 |
| | W95 | Val110Ile, Val190Ile, Tyr71Cys, | | | | |
| | W72 | Asp137Cys, Gly146Cys, Gly153Cys | | | | |
| | CC8 | E320A, Lys80Met, Ser138Ala, | 5 | CC45 | Val53Leu, | 20 |
| | W95 | Val110Ile, Val190Ile, Tyr71Cys, | | | Glu45Cys, | |
| | W72 | Asp137Cys, Gly146Cys, Gly153Cys | | | Thr122Cys, | |
| | | | | | Glu110Cys, | |
| | | | | | Arg155Cys | |
| CC8delta10 | CC8 | Deletion of C-terminal residues 312-321 | 64 | CC8 | none | 15 |
| CC45delta10 | CC45 | Deletion C-terminal residues 313-322 | 65 | CC45 | none | 16 |
| CC8 toxin | CC8 | none | 1 | CC8 | none | 15 |
| CC45 toxin | CC45 | none | 2 | CC45 | none | 16 |

### Example 2—Cytotoxicity of Wild-type, LukA, and LukAB Toxoids

The cytotoxicity of LukAB toxoid proteins (as defined in Table 4) was assessed in comparison with wild-type LukAB toxin using either the promonocytic cell line THP-1, or freshly isolated primary human polymorphonuclear leukocytes (hPMNs).

THP-1 cells were differentiated in the presence of phorbol 12-myristate 13-acetate prior to testing cytotoxicity. For THP-1 cytotoxicity assays, a total of 1×10⁵ cells in 50 μL RPMI were added to each well of a 96-well plate. LukAB toxins and toxoid proteins were adjusted to a standard concentration of protein, serially diluted in ice-cold RPMI medium, and 50 μL volumes of each were added to appropriate wells. In addition to RPMI-only negative controls, Triton X-100 was added to a final concentration of 0.1% as a positive control. Plates were incubated for 2 hours at 37° C., 5% CO₂, prior to assessing release of the cytoplasmic enzyme lactate dehydrogenase, which served as a marker of membrane integrity, using the CytoTox-ONE assay (Promega).

The cytotoxicity of LukA and LukAB toxoids against differentiated THP-1 cells is provided in Table 5 below. Differentiated THP-1 cells were sensitive to the wild-type toxins, as both the CC8 and CC45 LukAB wild-type toxins killed 30% or more of the cell population at toxin concentrations as low as 0.313 pg/mL. Deletion of the final 10 amino acid residues in the C-terminus of LukA (delta10) reduced the cytotoxicity of the CC8delta10 toxin to less than 5% cell death at 40 μg/mL but did not reduce the cytotoxicity of the CC45delta10 toxin toward differentiated THP-1 cells. Neither of the LukA monomers displayed cytotoxicity toward differentiated THP-1 cells. This result was expected, as LukA should not form an active pore complex in the absence of LukB. Each of the LukAB dimer toxoids, including RARPR-33, RARPR-34, and RARPR-15, displayed markedly reduced cytotoxicity toward differentiated THP-1 cells, with cell death at 1% or less for each of the toxoids tested at the highest tested concentration, 40 ug/mL.

TABLE 5
Cytotoxicity of LukA or LukAB proteins against differentiated THP-1 cells, a human
monocytic cell line, using a standardized amount of toxin. Data are presented as the percent of dead cells.

| | LukAB Concentration (μg/mL) | | | | | | | | | | | |
|---|---|---|---|---|---|---|---|---|---|---|---|---|
| Toxoid/Toxin | 40 | 20 | 10 | 5 | 2.5 | 1.25 | 0.625 | 0.313 | 0.156 | 0.078 | 0.04 | 0.02 |
| RARPR-15 | −12 | −12 | −12 | −14 | −14 | −15 | −10 | −14 | −12 | −11 | −9 | −7 |
| RARPR-30 | 0 | −1 | 0 | 0 | 0 | 0 | 0 | 0 | 0 | 0 | 0 | 0 |
| RARPR-31 | 0 | 0 | −1 | 0 | 0 | 0 | −1 | −1 | 0 | 0 | −1 | −1 |
| RARPR-32 | 0 | −1 | −1 | −1 | 0 | 0 | 0 | 0 | 0 | 0 | 0 | 0 |
| RARPR-33 | 1 | 1 | 1 | 0 | 1 | 1 | 1 | 0 | 1 | 1 | 1 | 1 |
| RARPR-34 | 1 | 0 | 1 | 1 | 1 | 1 | 0 | 1 | 1 | 0 | 0 | 1 |
| CC8 LukA W97 | 1 | 1 | 0 | 0 | 1 | 0 | 0 | 0 | 0 | 0 | 1 | 1 |
| (monomer) | | | | | | | | | | | | |
| CC45 LukA W97 | 1 | 1 | 0 | 0 | 0 | 0 | 0 | 0 | 0 | 1 | 1 | 2 |
| (monomer) | | | | | | | | | | | | |
| CC8delta10 | 4 | 3 | 1 | 1 | 0 | 0 | 0 | 0 | 0 | −1 | 0 | 0 |
| CC45delta10 | 53 | 39 | 32 | 31 | 33 | 41 | 54 | 55 | 53 | 35 | 13 | 5 |
| CC8 toxin | 41 | 44 | 43 | 39 | 39 | 38 | 37 | 30 | 15 | 6 | 2 | 1 |
| CC45 toxin | 42 | 34 | 31 | 34 | 40 | 46 | 46 | 36 | 18 | 4 | 1 | 0 |

For hPMNs, prior to intoxication, all toxins were normalized to 2.5 μg/mL (per subunit) and then 20 μL of toxin was pipetted into the top wells of a 96-well plate and serially diluted 2-fold in 10 μL of 1×PBS. PMNs were isolated and normalized to 200,000 cells per 90 μL RPMI (10 mM HEPES+0.1% HSA). 90 μL of PMNs were then pipetted into each well and the toxin-PMN mixture was incubated in a 37° C.+5% CO₂ incubator for 1 hour. To assess toxicity, 10 μL of CellTiter 96 Aqueous One Solution (CellTiter; Promega) was added to the 96-well plate, and the mixture was incubated at 37° C. in 5% CO2 for 1.5 hours. PMN viability was assessed with a PerkinElmer EnVision 2103 Multilabel Reader at an absorbance of 492 nm.

The cytotoxicity of LukA monomers and LukAB dimer toxoids against human primary PMN cells is provided in Table 6 below. The wild-type CC8 and CC45 toxins displayed greater than 90% killing of primary human PMNs at toxin concentrations of 0.313 μg/mL and 1.25 μg/mL, respectively. In comparison, each of the LukAB toxoids and the LukA monomers were considerably reduced in cytotoxicity toward these cells. Deletion of the 10 C-terminal residues in CC8 LukA essentially eliminated cytotoxicity toward differentiated THP-1 cells, whereas this toxin retained cytotoxicity against hPMNs, with greater than 20% killing observed at concentrations equal to or higher than 5 μg/mL. The CC8 and CC45 LukA monomers displayed little cytotoxicity toward hPMNs, as expected for toxoids lacking the LukB component critical for the formation of the active pore complex. Each of the LukAB dimer toxoids displayed notably reduced cytotoxicity toward hPMN cells in comparison with the CC8 and CC45 wild-type LukAB toxins. The RARPR-33 LukAB toxoid, as well as related toxoids RARPR-32 and -34, displayed less cytotoxicity than CC8delta10, with RARPR-33 killing only 15% of the cell population at the highest tested concentration, 20 ug/mL.

TABLE 6
Cytotoxicity of LukA or LukAB proteins against human primary polymorphonuclear
leukocytes using a standardized amount of toxin. Data are presented as the percent of dead cells

| | LukAB Concentration (μg/mL) | | | | | | | | | | |
|---|---|---|---|---|---|---|---|---|---|---|---|
| Toxoid/Toxin | 20 | 10 | 5 | 2.5 | 1.25 | 0.625 | 0.313 | 0.156 | 0.078 | 0.04 | 0.02 |
| RARPR-15 | 42 | 36 | 31 | 20 | 14 | 11 | 11 | 11 | 6 | 10 | 11 |
| RARPR-30 | 27 | 17 | 14 | 10 | 5 | 0 | 0 | 0 | 0 | 0 | 0 |
| RARPR-31 | 12 | 2 | 1 | 0 | 0 | 3 | 0 | 0 | 0 | 0 | 0 |
| RARPR-32 | 17 | 6 | 4 | 4 | 2 | 1 | 0 | 0 | 0 | 0 | 0 |
| RARPR-33 | 15 | 8 | 6 | 3 | 1 | 0 | 0 | 0 | 0 | 0 | 0 |
| RARPR-34 | 16 | 9 | 5 | 3 | 0 | 0 | 0 | 0 | 0 | 0 | 0 |
| CC8 LukA W97 | 5 | 3 | 2 | 1 | 2 | 1 | 1 | 2 | 0 | 2 | 1 |
| (monomer) | | | | | | | | | | | |
| CC45 LukA W97 | 7 | 6 | 2 | 3 | 4 | 1 | 0 | 1 | 2 | 0 | 0 |
| (monomer) | | | | | | | | | | | |
| CC8delta10 | 29 | 26 | 22 | 18 | 11 | 11 | 4 | 4 | 3 | 4 | 3 |
| CC8 toxin | 93 | 96 | 97 | 96 | 97 | 93 | 90 | 87 | 75 | 52 | 29 |
| CC45 toxin | 97 | 97 | 97 | 97 | 96 | 85 | 72 | 57 | 49 | 30 | 19 |

### Example 3—Cytotoxicity of RARPR-33 LukAB Toxoids and Other Variants of WT LukAB Toxins

An additional experiment was performed to evaluate the cytotoxicity and immunogenicity of RARPR-33 and different variants of WT LukAB toxin and LukA monomers. Mice were used for the immunogenicity studies.

The cytotoxicity of LukAB toxins, toxoids, and monomers was assessed on human PMNs. Prior to intoxication, all toxins were normalized to 100 μg/mL (per subunit) and then 20 μL of toxin was pipetted into the top wells of a 96-well plate and serially diluted 2-fold in 10 μL of 1×PBS. PMNs were isolated from different donors and normalized to 200,000 cells per 90 μL RPMI (10 mM HEPES+0.1% HSA). 90 μL of PMNs were pipetted into each well and the toxin-PMN mixture was incubated in a 37° C.+5% CO2 incubator for 1 hour. To assess toxicity, 10 μL of CellTiter 96 Aqueous One Solution (CellTiter; Promega) was added to the 96-well plate, and the mixture was incubated at 37° C. in 5% CO2 for 1.5 hours. PMN viability was assessed with a PerkinElmer EnVision 2103 Multilabel Reader at an absorbance of 492 nm. The percentage of dead cells was calculated by subtracting out background (healthy cells+PBS) and normalizing to Triton X100-treated cells which are set at 100% dead.

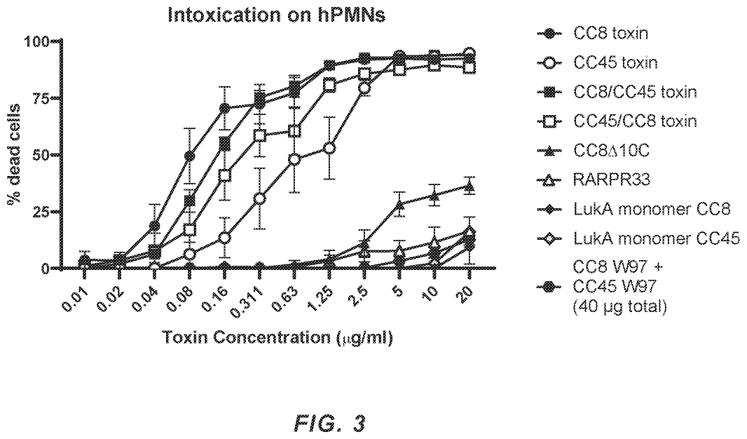

The cytotoxicity of LukA monomers and LukAB toxins against human primary PMN cells is provided in FIG. 3. The wild-type LukAB CC8 and CC45 toxins displayed greater than 90% killing of primary human PMNs at toxin concentrations of 2.5 μg/mL and 5 μg/mL, respectively. Maximum killing was also observed for the LukAB hybrid toxins CC8/CC45 and CC45/CC8 at 2.5 μg/mL. In comparison, the LukAB toxoids and the LukA monomers were considerably reduced in cytotoxicity toward these cells. Deletion of the 10 C-terminal residues in CC8 LukA retained cytotoxicity against hPMNs, with greater than 20% killing observed at concentrations equal to or higher than 5 μg/mL. The CC8 and CC45 LukA monomers and the combination of these monomers displayed little cytotoxicity toward hPMNs. The RARPR-33 LukAB toxoid displayed less cytotoxicity than CC8A10C, with RARPR-33 killing only 15% of the cell population at the highest tested concentration, 20 μg/mL.

### Example 4—Immunogenicity in Mice

To determine immunogenicity of the different LukAB variants, Envigo Hsd:ND4 (4 week old) mice (n=5/antigen) were subcutaneously administered 20 μg of LukAB in 50 μl of 10% glycerol 1×TBS mixed with 50 μl of the adjuvant, TiterMax® Gold. A cohort of 5 mice also received a mock immunization consisting of an equal volume of 10% glycerol 1×TBS and TiterMax® Gold. Following two boosts of the same antigen-adjuvant cocktail, with two weeks apart, mice were bled via cardiac puncture and serum was obtained.

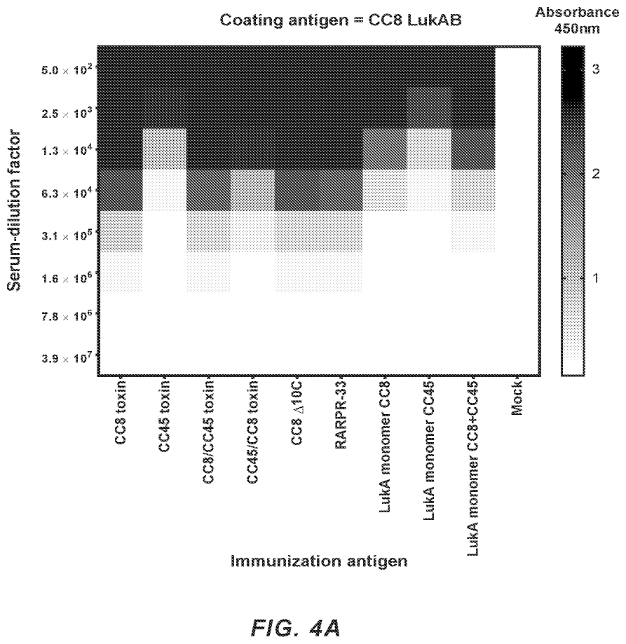

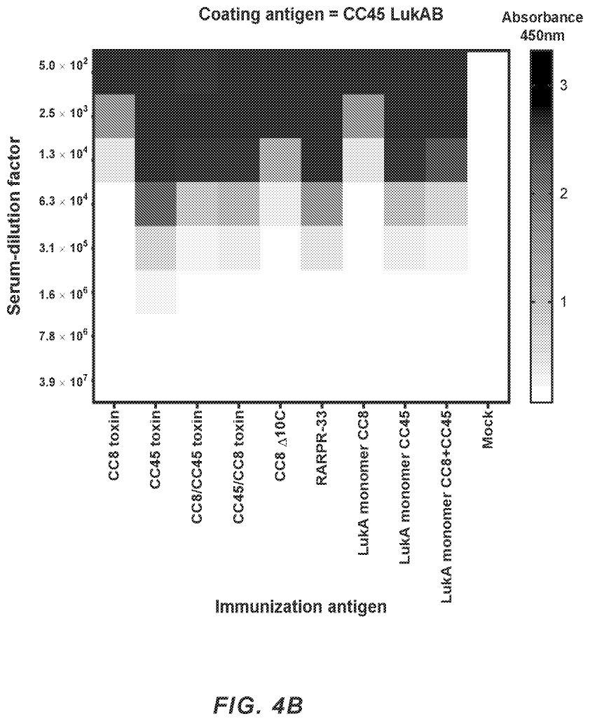

To determine anti-LukAB antibody titers, ELISAs were performed. WT LukAB CC8 or CC45 was diluted to 2 μg/ml in 1×PBS and coated in 100 μl in 96 well Immulon 21113 plates (Thermo Fisher, cat no. 3455) and incubated at 4° C. overnight. Plates were then washed 3× with wash buffer (1×PBS+0.05% Tween) and then blocked with 200 μl of blocking buffer (2.5% milk in 1×PBS) for 1 hr. Five-fold serial dilutions starting at 1:500 of serum into blocking buffer were generated and allowed to incubate on the rocker for 1 hr. The plates were then washed again 3× and mouse IgG-HRP (Biorad) antibody diluted 1:5,000 in blocking buffer was added and allowed to incubate for 1 hr at room temperature. Unbound secondary antibody was washed out in three successive washes with wash buffer. TMB (100 μl) that was brought up to room temperature was added to each well and incubated covered for 25 mins. After the reaction was completed, an equal amount of 2N Sulfuric acid was then added to each reaction well to stop the reaction. The plates were then read on an Envision plate reader for 450 nm absorbance. The heatmaps depicted in FIGS. 4A and 4B show average absorbance values from duplicate measurements, with black representing high absorbance and antibody binding to the coating antigen and white representing low absorbance and no antibody binding.

RARPR-33 elicited robust anti-CC8 and anti-CC45 LukAB IgG antibody titers. (FIG. 4A and FIG. 4B). RARPR-33 immunization elicited comparable anti-CC8 IgG responses as immunization with the CC8 WT toxin, CC8/CC45 hybrid toxin and the CC8A10C toxoid. The anti-CC8 LukAB IgG titers induced by the individual CC8 LukA monomer were not as high as those induced by CC8 LukAB toxin or the CC8/CC45 hybrid toxin, CC45/CC8 hybrid toxin, and RARPR 33 hybrid antigens (FIG. 4A).

The anti-CC45 LukAB titers in RARPR-33 immunized mice were higher than those elicited by the CC8/CC45 WT hybrid antigen and were on par with those elicited by the CC45 WT antigen. Combining the CC8 and CC45 LukA monomers elicited antibody titers to both CC8 and CC45 LukAB (FIG. 4B). However, these anti-CC8 and anti-CC45 LukAB titers elicited by the combined CC8 and CC45 LukA monomers were not as high as those elicited by RARPR 33. The individual CC45 LukA monomer elicits very high anti-CC45 LukAB titers—similar to the levels elicited by the CC45/CC8 hybrid and only slightly lower than those elicited by RARPR-33 or CC45 WT toxin. These results show that upon RARPR-33 immunization antibody responses towards both LukAB CC8 and CC45 are induced that are high in magnitude.

### Experiment 5—Antibody Mediated Neutralization of Toxin Cytotoxicity

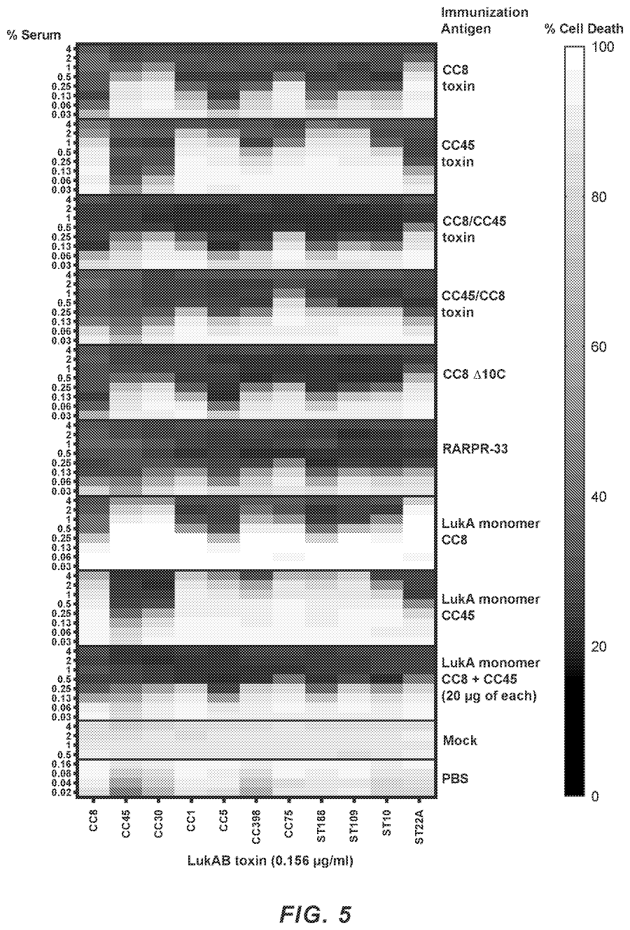

Antibody mediated neutralization of toxin cytotoxicity was assessed with serum obtained from mice immunized as described above in Example 4. Heat-inactivated pooled sera was normalized to 40% serum in PBS and then 20 μL of serum was pipetted into the top wells of a 96-well plate and serially diluted 2-fold in 10 μL of 1×PBS. An LD₉₀ of each of the LukAB toxin clonal complex sequence variants were added to the plate (10 μL/well) for 15 min at room temperature. Freshly isolated human primary polymorphonuclear leukocytes (hPMNs) normalized to 200,000 cells per 80 μL RPMI (10 mM HEPES+0.1% HSA) were then added to the serum-toxin mixture and incubated for 1 hr at 37° C.+5% CO2. To assess toxicity, 10 μL of CellTiter 96 Aqueous One Solution (CellTiter; Promega) was added to the 96-well plate, and the mixture was incubated at 37° C. in 5% CO2 for 1.5 hours. PMN viability was assessed with a PerkinElmer EnVision 2103 Multilabel Reader at an absorbance of 492 nm. The antibody neutralization data is presented FIG. 5.

Sera from mice immunized with RARPR-33 exhibited the most potent, broadly LukAB-neutralizing capacity of all the antigens (FIG. 5). The sera from RARPR 33-immunized mice strongly neutralized the cytotoxic effect of all 11 LukAB variants tested at as low as 0.25% serum, and for most LukAB variants also provided protection at as low as 0.063-0.125% serum (FIG. 5). Immunization with the individual CC8 and CC45 LukA monomers resulted in sera with LukAB-neutralizing capacity that is highly biased to the antigen backbone (FIG. 5). The combination of CC8 LukA monomer with CC45 LukA administered at 20 μg of each monomer (40 μg of total protein) for each immunization yielded sera with both broad and potent LukAB-neutralizing capacity, neutralizing all 11 LukAB variants tested at as low as 0.5% serum (FIG. 5), however, this is lower than to what was observed with sera from RARPR-33 immunized mice.

Combined, the data presented in Examples 3-5 show that the attenuating and stabilizing mutations incorporated into the CC8/CC45 LukAB backbone of RARPR-33 improves the broad immunogenic effects of the CC8/CC45 WT LukAB hybrid (FIGS. 4 and 5) while also rendering RARPR-33 highly attenuated compared to the CC8/CC45 WT LukAB toxin (FIG. 3).

### Example 6—Antisera Toxin Neutralization

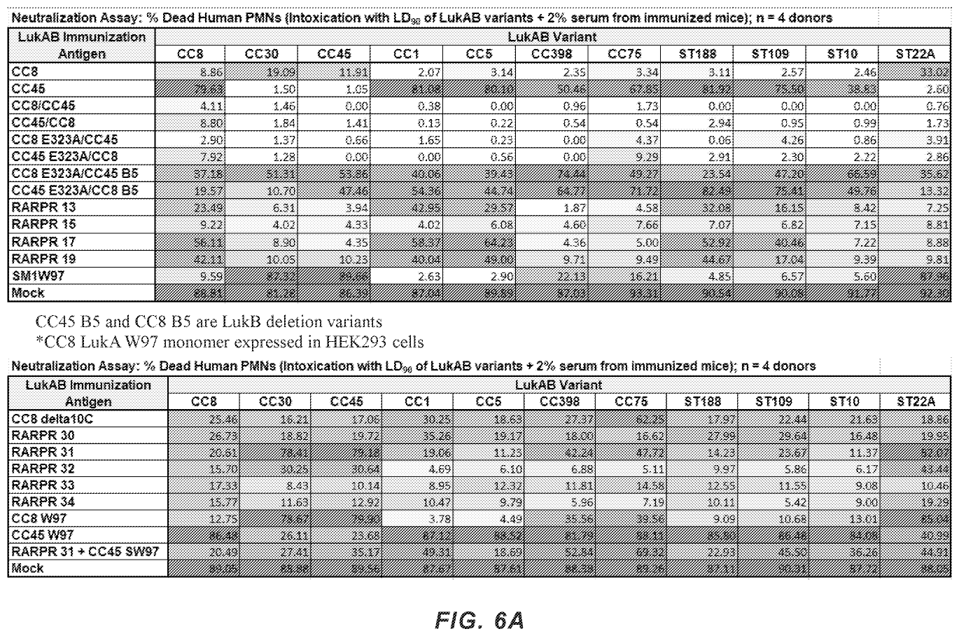

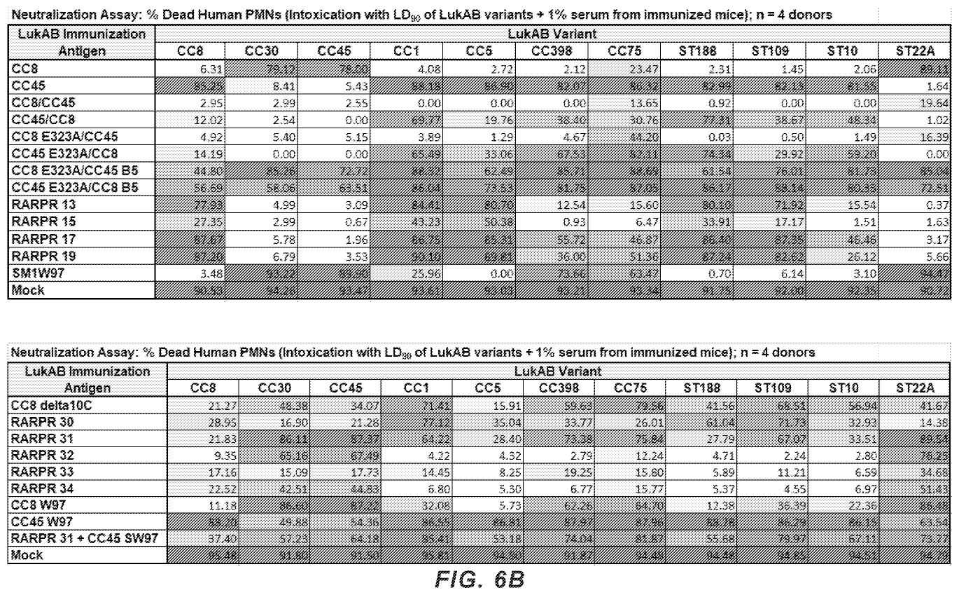

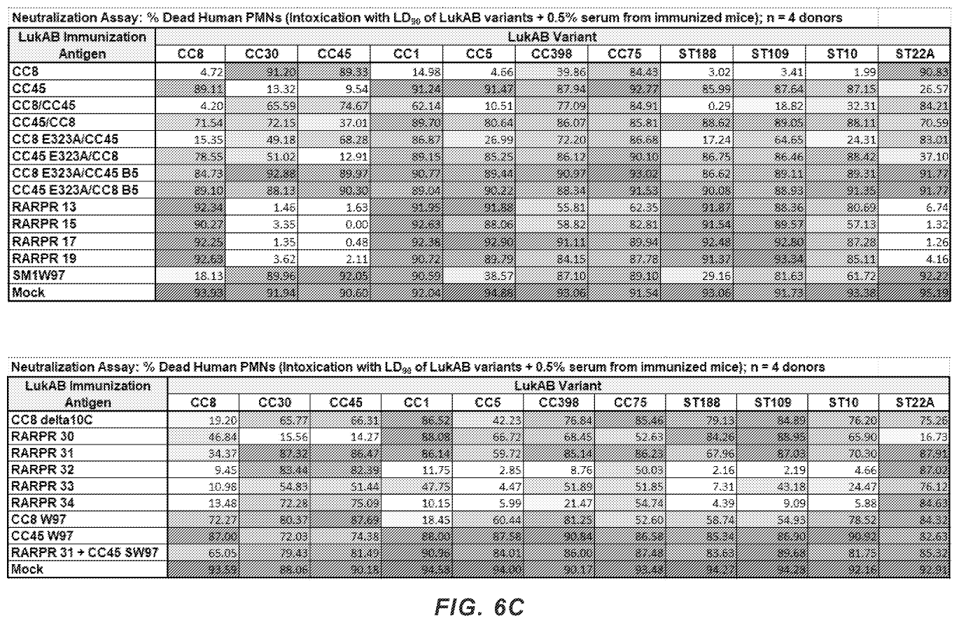

Antibody mediated neutralization of toxin cytotoxicity was assessed with serum obtained from mice immunized with wild-type LukAB, wild-type LukAB hybrids (i.e., CC8 LukA/CC45 LukB and CC45 LukA/CC8 LukB), LukA monomers, or LukAB toxoids. Heat-inactivated pooled sera were normalized to 40% serum in PBS and then 20 μL of serum was pipetted into the top wells of a 96-well plate and serially diluted 2-fold in 10 μL of 1×PBS. An LD₉₀ of each of the LukAB toxin clonal complex sequence variants were then added to the wells of the plate (10 μL/well) containing either 2%, 1% or 0.5% serum for 15 min at room temperature. Freshly isolated human primary polymorphonuclear leukocytes (hPMNs) from different donors were normalized to 200,000 cells per 80 μL RPMI (10 mM HEPES+0.1% HSA) were then added to the serum-toxin mixture and incubated for 1 hr at 37° C.+5% CO₂. To assess toxicity, 10 μL of CellTiter 96 Aqueous One Solution (CellTiter; Promega) was added to the 96-well plate, and the mixture was incubated at 37° C. in 5% CO₂ for 1.5 hours. PMN viability was assessed with a PerkinElmer EnVision 2103 Multilabel Reader at an absorbance of 492 nm. The antibody neutralization data is presented in the Tables of FIG. 6A (2% antibody serum), FIG. 6B (1% antibody serum, and FIG. 6C (0.5% antibody serum).

Immunization with wild-type CC8 and CC45 LukAB elicited antibodies that neutralized the naturally occurring sequence variants of LukAB toxins in a pattern that reflected the sequence composition of the immunized antigen. Antibodies elicited by CC8 LukAB toxin potently neutralized toxins derived from CC8, CC1, CC5, and other S. aureus lineages, but they did not provide complete neutralization of toxins derived from CC30, CC45, or ST22A S. aureus. Likewise, immunization with CC45 LukAB toxin elicited antibodies that potently neutralized toxins derived from CC30, CC45, or ST22A S. aureus lineages, but not toxins derived from other lineages.

Immunization of mice with a non-natural hybrid LukAB, either CC8 LukA combined with CC45 LukB or CC45 LukA combined with CC8 LukB, elicited antibodies that displayed broader neutralization of LukAB sequence variants in comparison with the naturally occurring dimer combinations. Of the non-natural hybrid dimers, CC8 LukA and CC45 LukB displayed a slightly better neutralization profile than the opposite combination, a pattern that was retained in proteins carrying the Glu to Ala substitution in the penultimate residue of LukA (E323A). As observed for antibodies elicited against the wild-type toxins, the LukA monomers elicited antibodies that displayed a neutralization pattern indicative of their sequence compositions. A combination of CC8 LukA and CC45 LukA monomers (RARPR-31+CC45 LukA W97) elicited antibodies that displayed a broad neutralizing pattern, but the potency of neutralization was reduced in comparison with the dimer antigens, as is evident by the reduced level of neutralization at 1% or 0.5% sera.

Of the dimer toxoids, RARPR-15, RARPR-33, and RARPR-34 displayed a broadly neutralizing antibody response against all tested LukAB sequence variants. The non-natural wild-type dimer combinations also displayed a broad neutralization profile, although the potency of the neutralizing response was inferior to that observed for several toxoids. Both the hybrid wild-type and the toxoid antigens displayed a broadly neutralizing profile when tested at 2% (FIG. 6A) and 1% (FIG. 6B) sera, but the improved potency of the response to the toxoids was evident when tested at 0.5% sera (FIG. 6C). At this lowest tested concentration, RARPR-15, RARPR-32 RARPR-33, and RARPR-34 each displayed a broad neutralizing response. RARPR-33, in particular, elicited sera that retained a broadly neutralizing response, whereas the hybrid wild-type antigens and the E323A toxoids failed to elicit a broadly protective response at 0.5% sera, and the neutralization pattern elicited by the CC45 toxoid RARPR-15 at the lowest tested concentration reflected its sequence composition, as high levels of neutralization were only observed for CC30, CC45, and ST22A LukAB toxins. The hybrid dimer toxoid RARPR-33 elicited a potent and broadly neutralizing immune response.

### Example 7—Cytotoxicity of RARPR-33 at High Concentrations

### Methods:

Cytotoxicity Assay:

To evaluate the cytotoxicity of each respective LukAB protein complex, freshly isolated primary human polymorphonuclear leukocytes (PMNs) were intoxicated with S. aureus toxins. PMNs were isolated from different donors and normalized to 200,000 cells per 50 μl RPMI (10 mM HEPES+0.1% HSA). 50 μl of toxin in PBS was added to the cells and the toxin-PMN mixture was incubated in a 37° C.+5% CO2 incubator for 1 hr. To assess toxicity, 10 μl of CellTiter 96 Aqueous One Solution (CellTiter; Promega) was added to the 96-well plate, and the mixture was incubated at 37° C. in 5% CO2 for 1.5 hrs. PMN viability was assessed with a PerkinElmer EnVision 2103 Multilabel Reader at an absorbance of 492 nm. % Dead cells are calculated by subtracting out background (healthy cells+PBS) and normalizing to TritonX-treated cells which are set at 100% dead.

LDH Assay:

To evaluate whether each respective LukAB protein complex can cause cell lysis, freshly isolated primary human polymorphonuclear leukocytes (PMNs) from different donors were intoxicated with S. aureus LukAB toxins and LDH release was measured. WT toxins were serially diluted 2-fold in PBS and tested at concentrations ranging from 5-0.0024 μg/ml. LukAB toxoids were diluted in PBS and tested at 2.5, 2, 1, 1.5, and 0.5 mg/ml. PMNs were isolated and normalized to 200,000 cells per 50 μl RPMI (10 mM HEPES+0.1% HSA). 50 μl of PMNs were then pipetted into each well and 50 μl of diluted toxin was added per well. The toxin-PMN mixture was incubated in a 37° C.+5% CO2 incubator for 2 hr. To assess LDH release, the plates were centrifuged at 1500 rpm for 5 min, then 25 μl of supernatant was removed from each well and transferred to 96-well black clear-bottom plates. 25 μl of CytoTox-ONE homogeneous membrane integrity reagent (Promega) was added to the black clear-bottom 96-well plate, and the mixture was incubated for 10 min at room temperature in the dark. Cell lysis was assessed with a PerkinElmer EnVision 2103 Multilabel Reader by recording fluorescence with an excitation wavelength of 560 nm and an emission wavelength of 590 nm. % Dead cells were calculated by subtracting out background (healthy cells+PBS) and normalizing to TritonX-treated cells which were set at 100% dead.

### Results:

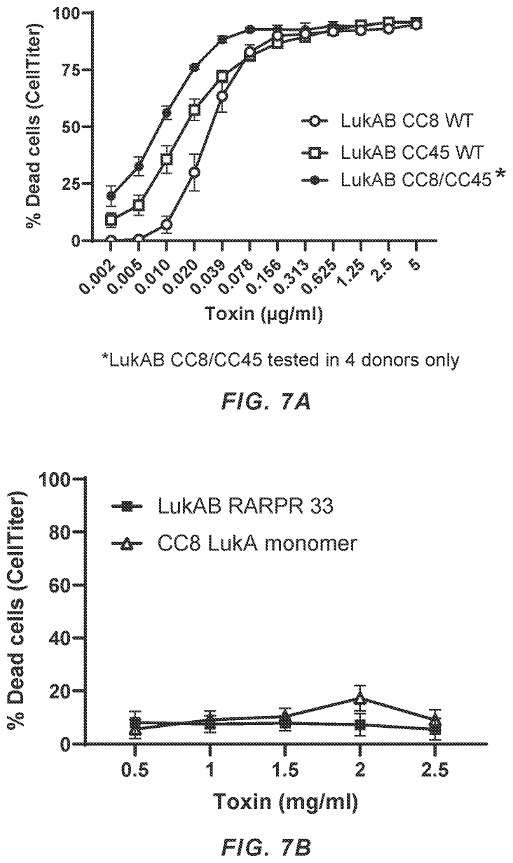

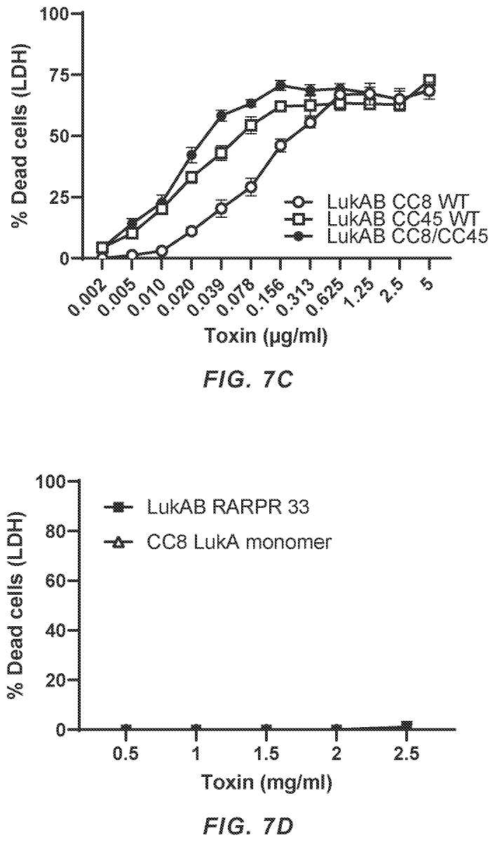

In previous examples, cytotoxicity of the LukAB toxoid RARPR-33 on hPMNs was determined up to a concentration of 20 μg/ml. Next, cytotoxicity of human PMN was monitored in presence of higher concentrations (up to 2.5 mg/ml) of RARPR-33. Maximum cytotoxicity of human PMNs (4-6 donors) based on CellTiter measurements was observed for the WT LukAB CC8, CC45 and CC8/CC45 toxins upon 1-hour intoxication with ˜0.156 μg/ml toxin (FIG. 7A). For RARPR-33 and the CC8 LukA monomer, the percentage of dead cells measured with CellTiter was ˜10% at a concentration of 0.5 mg/ml (FIG. 7B). Incubating PMNs with concentrations up to 2.5 mg/ml of RARPR-33 or the CC8 LukA monomer did not further increase the percentage of dead cells determined by CellTiter measurements.

The LD₁₅ value indicates the concentration of an antigen which induces 15% cell death. The LD₁₅ was determined using linear regression. For CC8 WT LukAB the LD₁₅ was 0.013 μg/ml, for CC45 WT LukAB the LD₁₅ was 0.004 μg/ml, and for CC8/CC45 LukAB hybrid the LD₁₅ was 0.002 μg/ml. The LD₁₅ for LukAB RARPR-33 was at 2.5 mg/ml. The LD₁₅ values were compared by dividing the LD₁₅ concentrations of RARPR-33 by the LD₁₅ concentration of the WT antigens. Based on these observations LukAB RARPR-33 toxicity is >192,308 fold less than LukAB CC8 WT, >625,000 fold less than LukAB CC45 WT, and >1,250,000 fold less than the LukAB CC8/CC45 hybrid.

In addition, a LDH assay was performed to assess plasma membrane damage after two hours of incubation with the different WT toxins, CC8 LukA monomer or RARPR-33. Cytotoxicity of human PMN was induced after 2 hours of exposure to WT toxins, CC8 WT, CC45 WT, or the CC8/CC45 toxin hybrid (FIG. 7C). Maximum cell death, determined by LDH, was observed at a concentration of 0.625 μg/ml toxin. In contrast, no plasma membrane damage of human PMNs was observed following two hours of exposure to RARPR-33 or the CC8 LukA monomer at concentrations up to 2.5 mg/ml (FIG. 7D). These data show that RARPR-33 is detoxified and unable to induce cell death of human PMNs at concentrations up to 2.5 mg/ml. The mutations incorporated into the CC8/CC45 LukAB backbone of RARPR-33, highly attenuated the cytotoxicity compared to the CC8/CC45 WT LukAB toxin.

### Example 8: Comparison of RARPR-33 vs D39A/R23E Toxoid

A LukAB toxoid based on a CC8 backbone was generated in which LukA has a D39A mutation and LukB has a R23E point mutation. This “D39A/R23E toxoid” was described in Kailasan, S. et al, “Rational Design of Toxoid Vaccine Candidates for Staphylococcus aureus Leukocidin AB (LukAB),” Toxins 11(6): (2019), which is hereby incorporated by reference in its entirety. This toxoid was generated on a LukAB CC8 backbone and was described to be >36,000-fold attenuated in toxicity as compared to WT CC8 LukAB toxin. The cytotoxicity was determined using the HL-60 cell line differentiated to be PMN-like. In the present experiment a comparison was made between the D39A/R23E toxoid and RARPR-33. The cytotoxicity on human polymorphonuclear leukocytes (PMNs) was determined and the ability to induce broadly toxin neutralizing antibodies upon immunization was assessed.

### Methods:

Cytotoxicity Assays:

To evaluate the cytotoxicity of each respective LukAB protein complex, freshly isolated primary human polymorphonuclear leukocytes (PMNs) from different donors were intoxicated with S. aureus toxins. PMNs were isolated and normalized to 200,000 cells per 50 μl RPMI (10 mM HEPES+0.1% HSA). To the cells, 50 μl of toxin in PBS was added and the toxin-PMN mixture was incubated in a 37° C.+5% CO₂ incubator for 2 hrs. To assess toxicity, 10 μl of CellTiter 96 Aqueous One Solution (CellTiter; Promega) was added to the 96-well plate, and the mixture was incubated at 37° C. in 5% CO₂ for 1.5 hrs. PMN viability was assessed with a PerkinElmer EnVision 2103 Multilabel Reader at an absorbance of 492 nm. The percentage of dead cells are calculated by subtracting out background (healthy cells+PBS) and normalizing to TritonX-treated cells which are set at 100% dead.

LDH Assay:

To evaluate whether each respective LukAB protein complex causes cell lysis, freshly isolated primary human polymorphonuclear leukocytes (PMNs) from different donors were intoxicated with S. aureus LukAB toxins and LDH release was measured. WT toxins were serially diluted 2-fold in PBS and tested at concentrations ranging between 0.5 μg/ml-0.00024 μg/ml. LukAB toxoids were diluted in PBS to a concentration ranging between 1 mg/ml-0.03125 mg/ml and tested. PMNs were isolated and normalized to 200,000 cells per 50 μl RPMI (10 mM HEPES+0.1% HSA). PMNs (50 μl) were then pipetted into each well and 50 μl of diluted toxin was added per well. The toxin-PMN mixtures were incubated in a 37° C.+5% CO₂ incubator for 2 hr. To assess LDH release, the plates were centrifuged at 1500 rpm for 5 min, then 25 μl of supernatant was removed from each well and transferred to 96-well black clear-bottom plates. 25 μl of CytoTox-ONE homogeneous membrane integrity reagent (Promega) was added to the black clear-bottom 96-well plate, and the mixture was incubated for 10 min at room temperature in the dark. Cell lysis was assessed with a PerkinElmer EnVision 2103 Multilabel Reader by recording fluorescence with an excitation wavelength of 560 nm and an emission wavelength of 590 nm. Percentage of dead cells was calculated by subtracting out background (healthy cells+PBS) and normalizing to TritonX-treated cells which were set at 100% death.

Mouse Immunizations.

Envigo Hsd:ND4 (4 week old) mice (n=5/antigen) were subcutaneously administered 20 μg of LukAB in 50 μl of 10% glycerol 1×TBS mixed with 50 μl of the adjuvant, TiterMax® Gold. Following two boosts of the same antigen/adjuvant cocktail, mice were bled via cardiac puncture and serum was obtained for toxin neutralization studies.

Toxin Neutralization Assay.

Sera from immunized mice was pooled from each group and heat inactivated in a water bath at 55° C. for 30 min. The pooled, heat-inactivated sera were then diluted to 40% with PBS. Further dilutions of the sera were then achieved by serially diluting the 40% stocks 2-fold in 10 μl of PBS in a 96 well plate. Toxin (10 l) was added into the serum wells at a final concentration of 0.156 μg/ml toxin (LD90). 80 μl of hPMNs at a concentration of 200,000 cells in RPMI+0.1% HSA+10 mM HEPES were added into each well. Plates were then incubated in a 37° C.+5% CO2 incubator for 1 hr. Following the incubation, Cell Titer was added to the intoxications and incubated for 1.5 hrs. Following the incubation, plates were then read on the plate reader at 492 nm absorbance. Percentages of dead cells were calculated by subtracting out background (healthy cells+PBS) and normalizing to TritonX-treated cells which are set at 100% death.

### Results:

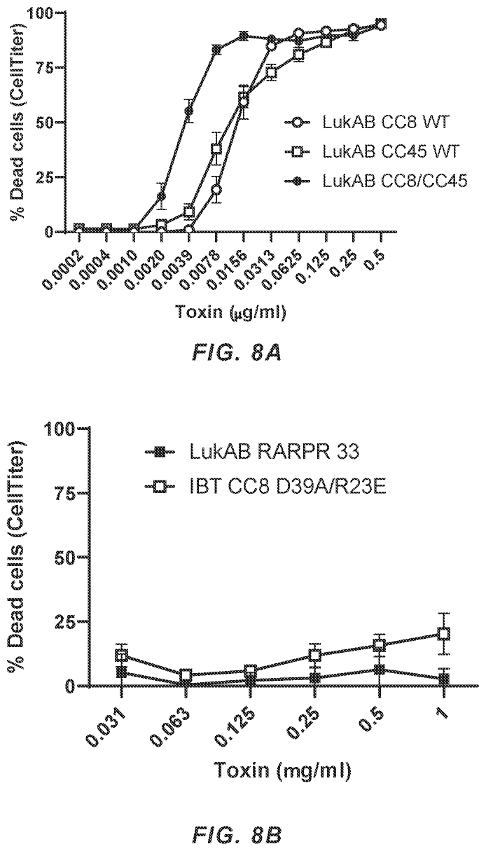

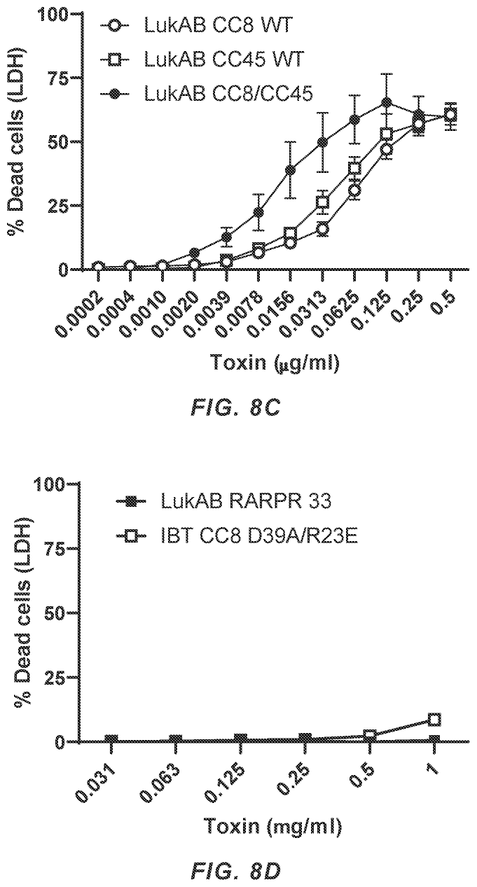

The cytotoxicity of the D39A/R23E toxoid has been reported to be tested up to ˜12 μg/ml. Here the cytotoxicity of RARPR-33 and the D39A/R23E toxoid were determined on human PMNs up to a concentration of 1 mg/ml. In addition, WT LukAB CC8, CC45 and CC8/CC45 were tested for comparison. Maximum cytotoxicity of human PMNs based on CellTiter measurements was observed upon 1-hour intoxication with ˜0.02 μg/ml WT LukAB CC8/CC45, ˜0.03 μg/ml LukAB CC8 and 0.125 μg/ml LukAB CC45 (FIG. 8A). The average of 5 donors is shown. For the D39A/R23E toxoid, around 22% of cytotoxicity was observed upon incubation with 1 mg/ml. For RARPR-33, the percentage of dead cells measured with CellTiter was around 3% at a concentration of 1 mg/ml (FIG. 8B).

In addition, a LDH assay was performed to assess plasma membrane damage after two hours of incubation with the different WT toxins, the D39A/R23E toxoid and RARPR-33. Cytotoxicity of human PMN was induced after 2 hours upon exposure to WT toxins, CC8 WT, CC45 WT and the combination of CC8/CC45 toxin hybrids (FIG. 8C). Maximum cell death, determined by LDH, was observed at a concentration of 0.25 μg/ml toxin. Upon two hours exposure of human PMN with concentrations up to 1 mg/ml of the D39A/R23E toxoid, around 8% of cell death was observed. When incubating human PMNs with a similar concentration of RARPR-33, no plasma membrane damage was observed, indicating no cell death (FIG. 8D). These results indicate that RARPR-33 is attenuated to below the limit of detection in the assay and is more attenuated than the D39A/R23E toxoid.

Sera from mice immunized with RARPR-33 or the D39A/R23E toxoid was tested in a toxin neutralization assay to assess the ability of the sera to prevent toxin induced cell death of human PMNs. Neutralization towards sixteen different LukAB toxins was tested on PMNs isolated from 4 donors.

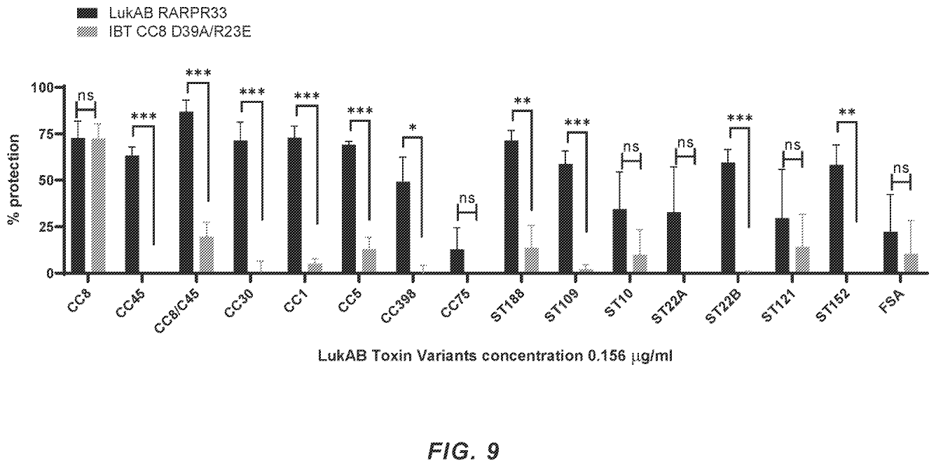

In the presence of 0.125% sera from RARPR 33-immunized mice, the cytotoxic effect of all 16 LukAB variants tested was neutralized (FIG. 9). At a similar serum concentration, sera from D39A/R23E toxoid immunized mice only protected equally as sera from RARPR-33 immunized mice against the cytotoxic effect of LukAB CC8. Against all other toxins, no protection or a much lower protection was observed for sera from D39A/R23E toxoid immunized mice. These results show that RARPR-33 immunization induced a much broader toxin neutralizing response than immunization with the D39A/R23E toxoid.

### Example 9: Thermal Stabilization of LukAB Toxoids

Stability of the LukAB toxoids in comparison to the wild-type protein was assessed through thermal unfolding experiments using intrinsic tryptophan or tyrosine fluorescence to estimate the melting temperature (Tm), corresponding to the midpoint of the transition of the protein from the folded to unfolded state. Thermal stability was assessed using the NanoTemper's PromethiusNT.Plex instrument (NanoTemper Inc., Germany). Thermal unfolding measurements were made on protein samples of 0.3 to 1 mg/mL (20 uL, buffer: 50 mM sodium phosphate buffer, 200 mM NaCl, pH 7.4, 10% glycerol) in duplicate runs for each sample. Prometheus NanoDSF user interface (Melting Scan tab) was used to set up the experimental parameters for the run. The thermal scans for a typical sample span from 20° C. to 95° C. at a rate of 1.0° C./min. A standard mAb (CNTO5825 or NIST) in the same buffer used for the samples was included as a control, and the runs were performed in duplicate. Thermal melting profiles were analyzed with the vendor software PR.ThermControl to determine the temperature at which 50% of the protein unfolds (Tm).

Tables 7A and 7B show the thermal stability of LukA and LukAB toxoid proteins as assessed by nanoDSF. The temperature of the start of protein unfolding (Tonset) and the midpoint of the transition (Tm1) of protein unfolding are presented, along with the difference in Tm between comparable constructs with and without stabilizing substitutions (ΔTm)

TABLE 7A
Single substitutions in the CC45 genetic background and combination
substitutions in the hybrid CC8/CC45 genetic background.

| Toxin | | | | | |
|---|---|---|---|---|---|
| Name | LukA | LukB | Tonset | Tm1 | ΔTmª |
| CC45 | CC45 LukA^{E321A} | CC45 LukB^{wt} | 40.3 | 47.3 | — |
| toxin | | | | | |
| CC8/ | CC8 LukA^{E320A} | CC45 LukB^{wt} | 37.7 | 43.9 | — |
| CC45 | | | | | |
| toxin | | | | | |
| | CC45 LukA^{E321A, Lys81Met} | CC45 LukB^{wt} | 40.5 | 47.4 | 0.1 |
| | CC45 LukA^{E321A, Ser139Ala} | CC45 LukB^{wt} | 40.5 | 47.4 | 0.1 |
| | CC45 LukA^{321A, Val111Ile} | CC45 LukB^{wt} | 40.0 | 47.7 | 0.4 |
| | CC45 LukA^{E321A, Val190Ile} | CC45 LukB^{wt} | 40.5 | 47.5 | 0.2 |
| | CC45 LukA^{E321A, Thr247Val} | CC45 LukB^{wt} | 41.3 | 47.3 | 0 |
| | CC45 LukA^{321A} | CC45 | 40.7 | 47.8 | 0.5 |
| | | LukB^{Val53Leu} | | | |
| RARPR- | CC45 LukA ^{E321A,} | CC45 LukB^{wt} | 41.2 | 48.9 | 1.6 |
| 15 | ^{Lys81Met, Ser139Ala,} | | | | |
| | ^{Val110Ile, Val191Ile} | | | | |
| RARPR- | CC8 LukA ^{E320A, Lys80Met,} | CC45 | 40.0 | 47.9 | 4.0 |
| 33 | ^{Ser138Ala, Val110Ile, Val190Ile} | LukB^{Val53Leu} | | | |
| LukA | CC8 LukA ^{E320A, Lys80Met,} | No LukB | 45.2 | 61.8 | — |
| monomer | ^{Ser138Ala, Val110Ile, Val190Ile,} | | | | |
| | ^{Thr246Val, Tyr71Cys, Asp137Cys,} | | | | |
| | ^{Gly146Cys, Gly153Cys} | | | | |
| LukA | CC45 LukA ^{E321A,} | No LukB | 51.9 | 58.1 | — |
| monomer | ^{Lys81Met, Ser139Ala,} | | | | |
| | ^{Val111Ile, Val191Ile,} | | | | |
| | ^{Thr247Val, Tyr72Cys, Asp138Cys,} | | | | |
| | ^{Gly147Cys, Gly154Cys} | | | | |

ªΔTm represents the difference between the Tm values for the CC45 or the CC8/CC45 toxins without stabilizing substitutions and disulfide bonds in comparison with the corresponding LukAB proteins carrying one or more substitutions.
LukA monomers included an N-terminal PelB signal sequence to direct expression to the periplasm of E. coli to support disulfide bond formation.
LukAB dimers carrying pairs of cysteine substitutions to support disulfide bond formation were expressed in the cytoplasm of E. coli Origami 2(DE3) cells.

TABLE 7B
Single substitutions in the CC8 genetic background and combination
substitutions in the hybrid CC8/CC45 genetic background.

| Toxin | | | | | |
|---|---|---|---|---|---|
| Name | LukA | LukB | Tonset | Tm1 | ΔTmª |
| CC45 | CC45 LukA^{E321A} | CC45 LukB^{wt} | 37.8 | 45.3 | — |
| toxin | | | | | |
| CC8/ | CC8 LukA^{E320A} | CC45 LukB^{wt} | 35.3 | 42.2 | — |
| CC45 | | | | | |
| toxin | | | | | |
| | CC8 LukA^{E320A, Lys80Met} | CC45 LukB^{wt} | 38.8 | 44.9 | 2.7 |
| | CC8 LukA^{E320A, Ser138Ala} | CC45 LukB^{wt} | 38.9 | 44.7 | 2.5 |
| | CC8 LukA^{E320A, Val110Ile} | CC45 LukB^{wt} | 38.0 | 44.0 | 1.8 |
| | CC8 LukA^{E320A, Val190Ile} | CC45 LukB^{wt} | 37.2 | 43.5 | 1.3 |
| | CC8 LukA^{E320A, Thr246Ile} | CC45 LukB^{wt} | 36.4 | 43.1 | 0.9 |
| | CC8 LukA ^{E320A, Lys80Met,} | CC45 LukB^{wt} | 40.5 | 46.8 | 4.6 |
| | ^{Ser138Ala, Val110Ile, Val190Ile,} | | | | |
| | ^{Thr246Val} | | | | |
| RARPR- | CC8 LukA ^{E320A, Lys80Met,} | CC45 | 38.8 | 46.3 | 4.1 |
| 33 | ^{Ser138Ala, Val110Ile, Val190Ile} | LukB^{Val53Leu} | | | |

Results:

Thermal stability analysis (Table 7A), revealed that the CC45 LukA^{E321A}/CC45 LukB protein displayed a Tm value 3° C. higher than the Tm for the CC8 LukA^{E321A}/CC45 LukB hybrid protein. Individual substitutions in CC45 LukA, in combination with CC45 LukB^{wt}, resulted in modest increases in Tm of 0 to 0.4° C. The Val53Leu substitution in LukB resulted in a 0.5° C. increase in Tm. As the hybrid LukAB toxoids included the CC8 LukA background, individual amino acid substitutions were tested in CC8 LukA, combined with wild-type CC45 LukB (Table 7B). As seen with CC45 LukA, the individual substitutions in CC8 LukA also increased Tm values above the wild-type LukAB. Combinations of substitutions in LukA (RARPR-15) produced a Tm value 1.6° C. higher than the CC45 LukA^{E321A}/CC45 LukB protein, and a combination of CC8 LukA substitutions with LukB^{Val53Leu} (RARPR-33) resulted in a Tm value that was 4° C. higher than the CC8 LukA^{E321A}/CC45 LukB hybrid. The increased thermal stability of RARPR-33 was observed in both datasets (Tables 7A and 7B). Although nanoDSF may produce some variability for proteins that unfold at less than 50° C., the ΔTm, determined using controls run within each set, was consistent across datasets at 4.0 and 4.1° C., respectively. The LukA monomers included both combinations of substitutions and pairs of cysteine substitutions and displayed elevated Tm values of ≥58° C., indicating the further contribution of disulfide bonds to increased thermal stability.

### Discussion of Examples 1-9

The stable LukAB variant heterodimer toxoids described herein possess several characteristics that render them highly suitable as S. aureus vaccine antigen candidate.

Firstly, the LukA monomers and LukAB dimer toxoids described herein, including RARPR-30, RARPR-31, RARPR-32, RARPR-33, RARPR-34, and RARPR-15, displayed markedly reduced cytotoxicity toward differentiated human THP-1 and human PMNs as compared to wildtype toxins and other known toxoids (i.e., CC8delta10 and CC45delta10 toxoids). Even at concentrations of up to 2.5 mg/ml, RARPR-33 remained non-cytotoxic, demonstrating its full attenuation.

Secondly, the combination of substitutions introduced in the LukA and LukB variant proteins significantly enhanced the thermal stability of the heterodimer RARPR complexes relative to corresponding toxoids containing only a single substitution. In particular, the combinations of substitutions in LukA (RARPR-15) produced a Tm value 1.6° C. higher than the CC45 LukA^{E321A}/CC45 LukB protein, and a combination of CC8 LukA substitutions with LukB^{Val53Leu} (RARPR-33) resulted in a Tm value that was 4° C. higher than the CC8 LukA^{E321A}/CC45 LukB hybrid.

In addition to the attenuated cytotoxicity and enhanced thermal stability, the LukAB RARPR toxoids described herein, particularly RARPR-15, RARPR-33, and RARPR-34 induced comparable or broader toxin neutralizing response and higher titers of neutralizing antibodies than wildtype CC45 and CC8 toxins, wildtype hybrid toxins, and toxoids, including the E323A toxoids and D39A/R23E toxoid.

In summary, the attenuated cytotoxicity, improved thermal stability, robust immunogenicity, and broadly neutralizing antibody profile renders the LukAB RARPR toxoids described herein ideal vaccine antigen candidates.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

1. A variant Staphylococcus aureus Leukocidin A (LukA) polypeptide of SEQ ID NO:25, said LukA variant polypeptide comprising:
an amino acid substitution at one or more amino acid residues corresponding to amino acid residues Lys83, Ser141, Val113, and Val193 of SEQ ID NO: 25.

2. The LukA variant polypeptide of claim 1, wherein said LukA variant polypeptide further comprises:
an amino acid substitution at the amino acid residue corresponding to Glu323 of SEQ ID NO: 25.

3. The LukA variant polypeptide of claim 2, wherein the amino acid substitution at the amino acid residue corresponding to Glu323 comprises a glutamic acid to alanine (Glu323Ala) substitution, the amino acid substitution at the amino acid residue corresponding to Lys83 comprises a lysine to methionine (Lys83Met) substitution, the amino acid substitution at the amino acid residue corresponding to Ser141 comprises a serine to alanine (Ser141Ala) substitution, the amino acid substitution at the amino acid residue corresponding to Val113 comprises a valine to isoleucine (Val113Ile) substitution, and/or the amino acid substitution at the amino acid residue corresponding to Val193 comprises a valine to isoleucine (Val193Ile) substitution.

4.-7. (canceled)

8. The LukA variant polypeptide of claim 2, wherein said LukA variant polypeptide comprises amino acid substitutions at each amino acid residue corresponding to amino acid residues Lys83, Ser141, Val113, Val193, and Glu323 of SEQ ID NO: 25.

9. The LukA variant polypeptide of claim 8, wherein the amino acid substitutions comprise Lys83Met, Ser141Ala, Val113Ile, Val193Ile, and Glu323Ala.

10. The LukA variant polypeptide of claim 1, wherein said variant is selected from:
(ii) a CC8 LukA variant of SEQ ID NO: 1 comprising amino acid substitutions corresponding to Lys80Met, Ser138Ala, Val110Ile, Val190Ile, and Glu320Ala in SEQ ID NO: 1, or wherein said LukA variant polypeptide comprises the amino acid sequence of SEQ ID NO: 3; and
(ii) a CC45 LukA variant of SEQ ID NO: 2 comprising amino acid substitutions corresponding to Lys81Met, Ser139Ala, Val111Ile, Val191Ile, and Glu321Ala in SEQ ID NO: 2, or wherein said LukA variant polypeptide comprises the amino acid sequence of SEQ ID NO: 4.

11.-13. (canceled)

14. The LukA variant polypeptide of claim 1, wherein said LukA variant polypeptide further comprises:
an amino acid substitution at one or more amino acid residues corresponding to amino acid residues Tyr74, Asp140, Gly149, and Gly156 of SEQ ID NO: 25.

15. The LukA variant polypeptide of claim 14, wherein the amino acid substitution at the amino acid residue corresponding to Tyr74 comprises a tyrosine to cysteine (Tyr74Cys) substitution, the amino acid substitution at the amino acid residue corresponding to Asp140 comprises an asparagine to cysteine (Asp140Cys) substitution, the amino acid substitution at the amino acid residue corresponding to Gly149 comprises a glycine to cysteine (Gly149Cys) substitution, and/or the amino acid substitution at the amino acid residue corresponding to Gly156 comprises a glycine to cysteine (Gly156Cys) substitution.

16.-18. (canceled)

19. The LukA variant polypeptide of claim 14, wherein said LukA variant polypeptide comprises amino acid substitutions at each amino acid residue corresponding to amino acid residues Tyr74, Asp140, Gly149, and Gly156 of SEQ ID NO: 25.

20. The LukA variant polypeptide of claim 14, wherein said variant is selected from:
(ii) a CC8 LukA variant of SEQ ID NO: 1 comprising amino acid substitutions corresponding to Lys80Met, Ser138Ala, Val110Ile, Val190Ile, Glu320Ala, Tyr71Cys, Asp137Cys, Gly146Cys, and Gly153Cys of SEQ ID NO: 1, or wherein said LukA variant polypeptide comprises the amino acid sequence of SEQ ID NO: 5; and
(ii) a CC45 LukA variant of SEQ ID NO: 2 comprising amino acid substitutions corresponding to Lys81Met, Ser139Ala, Val111Ile, Val191Ile, Glu321Ala, Tyr72Cys, Asp138Cys, Gly147Cys, and Gly154Cys of SEQ ID NO: 2, or wherein said LukA variant polypeptide comprises the amino acid sequence of SEQ ID NO: 6.

21.-23. (canceled)

24. The LukA variant polypeptide of claim 1, wherein said LukA variant polypeptide further comprises:
an amino acid substitution at the amino acid residue corresponding to amino acid residue Thr249 of SEQ ID NO: 25.

25. The LukA variant polypeptide of claim 24, wherein
the amino acid substitution at the amino acid residue corresponding to Thr249 comprises a threonine to valine (Thr249Val) substitution, or wherein said variant LukA protein comprises the amino acid sequence of SEQ ID NO: 7 or SEQ ID NO: 8.

26. (canceled)

27. The LukA variant polypeptide of claim 1 further comprising: an amino-terminus signal sequence or an amino-terminus purification sequence.

28. (canceled)

29. (canceled)

30. A nucleic acid molecule encoding the LukA variant polypeptide of claim 1.

31. An expression vector comprising the nucleic acid molecule of claim 30.

32. A host cell comprising the expression vector of claim 31.

33. A variant Staphylococcus aureus Leukocidin B (LukB) protein or polypeptide of SEQ ID NO: 39, said LukB variant polypeptide comprising:
an amino acid substitution at the amino acid residue corresponding to amino acid residue Val53 of SEQ ID NO: 39.

34. The LukB variant polypeptide of claim 33, wherein the amino acid substitution at the amino acid residue corresponding to Val53 comprises a valine to leucine (Val53Leu) substitution.

35. The LukB variant polypeptide of claim 33, wherein said variant is selected from:
(i) a CC8 LukB variant of SEQ ID NO: 15 comprising the amino acid substitution corresponding to Val53Leu of SEQ ID NO: 15, or wherein said LukB variant polypeptide comprises the amino acid sequence of SEQ ID NO: 17; and
(ii) a CC45 LukB variant of SEQ ID NO: 16 comprising the amino acid substitution corresponding to Val53Leu of SEQ ID NO: 16, or wherein said LukB variant polypeptide comprises the amino acid sequence of SEQ ID NO: 18.

36.-38. (canceled)

39. The LukB variant polypeptide of claim 33, wherein said variant further comprises an amino acid substitution at one or more amino acid residues corresponding to amino acid residues Glu45, Glu109, Thr121, and Arg154 of SEQ ID NO: 39.

40. The LukB variant polypeptide of claim 39, wherein the amino acid substitution at the amino acid residue corresponding to Glu45 comprises a glutamic acid to cysteine (Glu45Cys) substitution, the amino acid substitution at the amino acid residue corresponding to Glu109 comprises a glutamic acid to cysteine (Glu109Cys) substitution, the amino acid substitution at the amino acid residue corresponding to Thr121 comprises a threonine to cysteine (Thr121Cys) substitution, and/or the amino acid substitution at the amino acid residue corresponding to Arg154 comprises an arginine to cysteine (Arg154Cys) substitution.

41.-43. (canceled)

44. The LukB variant polypeptide of claim 39, wherein said LukB variant polypeptide comprises amino acid substitutions at each amino acid residue corresponding to amino acid residues Glu45, Glu109, Thr121, and Arg154 of SEQ ID NO: 39.

45. The LukB variant polypeptide of claim 33, wherein said variant is selected from:
(i) a CC8 LukB variant of SEQ ID NO: 15 comprising the amino acid substitutions corresponding to Val53Leu, Glu45Cys, Glu109Cys, Thr121Cys, and Arg154Cys of SEQ ID NO: 15 or wherein said LukB variant polypeptide comprises the amino acid sequence of SEQ ID NO: 19; and
(ii) a CC45 LukB variant of SEQ ID NO: 16 comprising the amino acid substitutions corresponding to Val53Leu, Glu45Cys, Glu110Cys, Thr123Cys, and Arg155Cys of SEQ ID NO: 16, or wherein said LukB variant polypeptide comprises the amino acid sequence of SEQ ID NO: 20.

46.-48. (canceled)

49. A variant Staphylococcus aureus Leukocidin B (LukB) protein or polypeptide of SEQ ID NO:39, said LukB variant polypeptide comprising:
an amino acid substitution at one or more amino acid residues corresponding to amino acid residues Glu45, Glu109, Thr121, and Arg154 of SEQ ID NO: 39.

50. The LukB variant polypeptide of claim 49, wherein the amino acid substitution at the amino acid residue corresponding to Glu45 comprises a glutamic acid to cysteine (Glu45Cys) substitution, the amino acid substitution at the amino acid residue corresponding to Thr121 comprises a threonine to cysteine (Thr121Cys) substitution, the amino acid substitution at the amino acid residue corresponding to Glu109 comprises a glutamic acid to cysteine (Glu109Cys) substitution, and/or the amino acid substitution at the amino acid residue corresponding to Arg154 comprises an arginine to cysteine (Arg154Cys) substitution.

51. (canceled)

52. The LukB variant polypeptide of claim 49, wherein said LukB variant polypeptide comprises amino acid substitutions at each amino acid residue corresponding to amino acid Glu45, Glu109, Thr121, and Arg154 of SEQ ID NO: 39.

53. The LukB variant polypeptide of claim 52, wherein said variant is selected from:
(i) a CC8 LukB variant of SEQ ID NO: 15 comprising the amino acid substitutions corresponding to Glu45Cys, Glu109Cys, Thr121Cys, and Arg154Cys of SEQ ID NO: 15, or wherein said LukB variant polypeptide comprises the amino acid sequence of SEQ ID NO: 21; and
(ii) a CC45 LukB variant of SEQ ID NO: 16 comprising the amino acid substitutions corresponding to Glu45Cys, Glu110Cys, Thr123Cys, and Arg155Cys of SEQ ID NO: 16, or wherein said LukB variant polypeptide comprises the amino acid sequence of SEQ ID NO: 22.

54.-56. (canceled)

57. The LukB variant polypeptide of claim 33 further comprising: an amino-terminus signal sequence or an amino-terminus purification tag.

58. (canceled)

59. (canceled)

60. A nucleic acid molecule encoding the LukB variant polypeptide of claim 33.

61. An expression vector comprising the nucleic acid molecule of claim 60.

62. An expression vector comprising:
the nucleic acid molecule of claim 30 operably coupled to a nucleic acid molecule encoding a LukB variant polypeptide.

63. A host cell comprising the expression vector of claim 61.

64. A Staphylococcus aureus vaccine composition comprising:
one or more variant LukA proteins or polypeptides of claim 1.

65. The vaccine composition of claim 64, wherein the LukA variant polypeptide is a variant of SEQ ID NO: 1 or SEQ ID NO: 2.

66. The vaccine composition of claim 64 further comprising:
a leukocidin B (LukB) protein or polypeptide, said LukB protein or polypeptide having at least 85% sequence similarity to the amino acid sequence of SEQ ID NO: 15; or
a leukocidin B (LukB) protein or polypeptide, said LukB protein or polypeptide having at least 85% sequence similarity to the amino acid sequence of SEQ ID NO: 16.

67. (canceled)

68. The vaccine composition of claim 66, wherein the LukA variant polypeptide comprises an amino acid substitution at one or more amino acid residues corresponding to amino acid residues Lys80Met, Ser138Ala, Val110Ile, Val190Ile, and Glu320Ala of SEQ ID NO: 1.

69. The vaccine composition of claim 68, wherein the LukA variant polypeptide comprises the amino acid sequence SEQ ID NO: 3 and the LukB polypeptide comprises the amino acid sequence of SEQ ID NO:18, SEQ ID NO: 15, or SEQ ID NO: 17.

70.-74. (canceled)

75. The vaccine composition of claim 65, wherein the LukA variant polypeptide comprises an amino acid substitution at one or more amino acid residues corresponding to amino acid residues Lys81Met, Ser139Ala, Val111Ile, Val191Ile and Glu321Ala of SEQ ID NO: 2.

76. The vaccine composition of claim 75, wherein the LukA variant polypeptide comprises the amino acid sequence SEQ ID NO: 4 and the LukB protein or polypeptide comprises the amino acid sequence of SEQ ID NO:16.

77. A Staphylococcus aureus vaccine composition comprising:
one or more variant LukB proteins or polypeptides of claim 33.

78. The vaccine composition of claim 77 further comprising:
a leukocidin A (LukA) protein or polypeptide, said LukA protein or polypeptide having at least 85% sequence similarity to the amino acid sequence of SEQ ID NO: 1 (CC8): or
a leukocidin A (LukA) protein or polypeptide, said LukA protein or polypeptide having at least 85% sequence similarity to the amino acid sequence of SEQ ID NO: 2 (CC45).

79. (canceled)

80. A Staphylococcus aureus vaccine composition comprising:
a LukA variant polypeptide of claim 1, and
a LukB variant polypeptide of SEQ ID NO: 39, said LukB variant polypeptide comprising:
(i) an amino acid substitution at the amino acid residue corresponding to amino acid residue Val53 of SEQ ID NO: 39; or
(ii) an amino acid substitution at one or more amino acid residues corresponding to amino acid residues Glu45, Glu109, Thr121, and Arg154 of SEQ ID NO: 39.

81.-82. (canceled)

83. A method of generating an immune response against S. aureus in a subject, said method comprising:
administering the vaccine composition of claim 68 to the subject under conditions effective to generate said immune response against S. aureus in said subject.

84. (canceled)

85. A nucleic acid molecule encoding the LukB variant polypeptide of claim 49.

86. A method of generating an immune response against S. aureus in a subject, said method comprising:
administering the vaccine composition of claim 66 to the subject under conditions effective to generate said immune response against S. aureus in said subject.

87. A method of generating an immune response against S. aureus in a subject, said method comprising:
administering the vaccine composition of claim 80 to the subject under conditions effective to generate said immune response against S. aureus in said subject.


SEQUENCE LISTING (65 sequences)

SEQ ID NO: 1 - protein, 321 aa, S. aureus
HKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 2 - protein, 322 aa, S. aureus
ANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKYSDQNKPYKEG

SEQ ID NO: 3 - protein, 321 aa, Artificial
  S. aureus LukA Variant
HKDSQDQNKKEHVDKSQQKDKRNVTNKDKNSTAPDDIGKNGKITKRTETVYDEKTNILQNLQFDFIDDPTYDKNVLLVKMQGSIHSNLKFESHKEEKNSNWLKYPSEYHIDFQVKRNRKTEILDQLPKNKISTAKVDATFSYSSGGKFDSTKGIGRTSSNSYSKTISYNQQNYDTIASGKNNNWHVHWSIIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKAG

SEQ ID NO: 4 - protein, 322 aa, Artificial
  S. aureus LukA Variant
ANKDSQDQTKKEHVDKAQQKEKRNVNDKDKNTPGPDDIGKNGKVTKRTVSEYDKETNILQNLQFDFIDDPTYDKNVLLVKMQGSIHSNLKFESHRNETNASWLKYPSEYHIDFQVQRNPKTEILDQLPKNKISTAKVDATFSYSLGGKFDSTKGIGRTSSNSYSKSISYNQQNYDTIASGKNNNRHVHWSIVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKYSDQNKPYKAG

SEQ ID NO: 5 - protein, 321 aa, Artificial
  S. aureus LukA Variant
HKDSQDQNKKEHVDKSQQKDKRNVTNKDKNSTAPDDIGKNGKITKRTETVYDEKTNILQNLQFDFIDDPTCDKNVLLVKMQGSIHSNLKFESHKEEKNSNWLKYPSEYHIDFQVKRNRKTEILDQLPKNKISTAKVCATFSYSSGCKFDSTKCIGRTSSNSYSKTISYNQQNYDTIASGKNNNWHVHWSIIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKAG

SEQ ID NO: 6 - protein, 322 aa, Artificial
  S. aureus LukA Variant
ANKDSQDQTKKEHVDKAQQKEKRNVNDKDKNTPGPDDIGKNGKVTKRTVSEYDKETNILQNLQFDFIDDPTCDKNVLLVKMQGSIHSNLKFESHRNETNASWLKYPSEYHIDFQVQRNPKTEILDQLPKNKISTAKVCATFSYSLGCKFDSTKCIGRTSSNSYSKSISYNQQNYDTIASGKNNNRHVHWSIVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKYSDQNKPYKAG

SEQ ID NO: 7 - protein, 321 aa, Artificial
  S. aureus LukA Variant
HKDSQDQNKKEHVDKSQQKDKRNVTNKDKNSTAPDDIGKNGKITKRTETVYDEKTNILQNLQFDFIDDPTYDKNVLLVKMQGSIHSNLKFESHKEEKNSNWLKYPSEYHIDFQVKRNRKTEILDQLPKNKISTAKVNATFSYSSGGKFDSTKGIGRTSSNSYSKTISYNQQNYDTIASGKNNNWHVHWSIIANDLKYGGEVKNRNDELLFYRNTRIATVENPELSFASKYRYPALVRSGFNPEFLVYLSNEKSNEKTQFEVTYTRNQDILKNRPGIHYAPPILEKNKDGQRLIVTYEVDWKNKTVKVVDKYSDDNKPYKAG

SEQ ID NO: 8 - protein, 322 aa, Artificial
  S. aureus LukA Variant
ANKDSQDQTKKEHVDKAQQKEKRNVNDKDKNTPGPDDIGKNGKVTKRTVSEYDKETNILQNLQFDFIDDPTYDKNVLLVKMQGSIHSNLKFESHRNETNASWLKYPSEYHIDFQVQRNPKTEILDQLPKNKISTAKVDATFSYSLGGKFDSTKGIGRTSSNSYSKSISYNQQNYDTIASGKNNNRHVHWSIVANDLKYGNEIKNRNDEFLFYRNTRLSTVENPELSFASKYRYPALVRSGFNPEFLVYISNEKTNDKTRFEVTYTRNQDILKNKPGIHYGQPILEQNKDGQRFIVVYEVDWKNKTVKVVEKYSDQNKPYKAG

SEQ ID NO: 9 - protein, 321 aa, Artificial
  S. aureus LukA Variant
HKDSQDQNKKEHVDKSQQKDKRNVTNKDKNSTAPDDIGKNGKITKRTETVYDEKTNILQNLQFDFIDDPTCDKNVLLVKMQGSIHSNLKFESHKEEKNSNWLKYPSEYHIDFQVKRNRKTEILDQLPKNKISTAKVCATFSYSSGCKFDSTKCIGRTSSNSYSKTISYNQQNYDTIASGKNNNWHVHWSIIANDLKYGGEVKNRNDELLFYRNTRIATVENPELSFASKYRYPALVRSGFNPEFLVYLSNEKSNEKTQFEVTYTRNQDILKNRPGIHYAPPILEKNKDGQRLIVTYEVDWKNKTVKVVDKYSDDNKPYKAG

SEQ ID NO: 10 - protein, 322 aa, Artificial
  S. aureus LukA Variant
ANKDSQDQTKKEHVDKAQQKEKRNVNDKDKNTPGPDDIGKNGKVTKRTVSEYDKETNILQNLQFDFIDDPTCDKNVLLVKMQGSIHSNLKFESHRNETNASWLKYPSEYHIDFQVQRNPKTEILDQLPKNKISTAKVCATFSYSLGCKFDSTKCIGRTSSNSYSKSISYNQQNYDTIASGKNNNRHVHWSIVANDLKYGNEIKNRNDEFLFYRNTRLSTVENPELSFASKYRYPALVRSGFNPEFLVYISNEKTNDKTRFEVTYTRNQDILKNKPGIHYGQPILEQNKDGQRFIVVYEVDWKNKTVKVVEKYSDQNKPYKAG

SEQ ID NO: 11 - protein, 322 aa, Artificial
  S. aureus LukA Variant
ANKDSQDQTKKEHVDKAQQKEKRNVNDKDKNTPGPDDIGKNGKVTKRTVSEYDKETNILQNLQFDFIDDPTYDKNVLLVKLQGSIHSNLKFESHRNETNASWLKYPSEYHIDFQVQRNPKTEILDQLPKNKISTAKVDATFSYSLGGKFDSTKGIGRTSSNSYSKSISYNQQNYDTIASGKNNNRHVHWSIVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKYSDQNKPYKAG

SEQ ID NO: 12 - protein, 322 aa, Artificial
  S. aureus LukA Variant
ANKDSQDQTKKEHVDKAQQKEKRNVNDKDKNTPGPDDIGKNGKVTKRTVSEYDKETNILQNLQFDFIDDPTYDKNVLLVKLQGSIHSNLKFESHRNETNASWLKYPSEYHIDFQVQRNPKTEILDQLPKNKISTAKVDATFSYSLGGKFDSTKGIGRTSSNSYSKSISYNQQNYDTIASGKNNNRHVHWSIVANDLKYGNEIKNRNDEFLFYRNTRLSTVENPELSFASKYRYPALVRSGFNPEFLVYISNEKTNDKTRFEVTYTRNQDILKNKPGIHYGQPILEQNKDGQRFIVVYEVDWKNKTVKVVEKYSDQNKPYKAG

SEQ ID NO: 13 - protein, 321 aa, Artificial
  S. aureus LukA Variant
HKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKAG

SEQ ID NO: 14 - protein, 322 aa, Artificial
  S. aureus LukA Variant
ANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKYSDQNKPYKAG

SEQ ID NO: 15 - protein, 309 aa, S. aureus
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 16 - protein, 310 aa, S. aureus
EIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTFNDKEKK

SEQ ID NO: 17 - protein, 309 aa, Artificial
  S. Aureus LukB variant
KINSEIKQVSEKNLDGDTKMYTRTATTSDSQKNITQSLQFNFLTEPNYDKETLFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 18 - protein, 310 aa, Artificial
  S. Aureus LukB variant
EIKSKITTVSEKNLDGDTKMYTRTATTSDTEKKISQSLQFNFLTEPNYDKETLFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTFNDKEKK

SEQ ID NO: 19 - protein, 309 aa, Artificial
  S. Aureus LukB variant
KINSEIKQVSEKNLDGDTKMYTRTATTSDSQKNITQSLQFNFLTCPNYDKETLFIKAKGTIGSGLRILDPNGYWNSTLRWPGSYSVSIQNVDDNNNTNVTDFAPKNQDCSREVKYTYGYKCGGDFSINRGGLTGNITKESNYSETISYQQPSYCTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 20 - protein, 310 aa, Artificial
  S. Aureus LukB variant
EIKSKITTVSEKNLDGDTKMYTRTATTSDTEKKISQSLQFNFLTCPNYDKETLFIKAKGTIGSGLKILNPNGYWNSTLRWPGSYSVSIQNVDDNNNSTNVTDFAPKNQDCSREVKYTYGYKCGGDFSINRGGLTGNITKEKNYSETISYQQPSYCTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTFNDKEKK

SEQ ID NO: 21 - protein, 309 aa, Artificial
  S. Aureus LukB variant
KINSEIKQVSEKNLDGDTKMYTRTATTSDSQKNITQSLQFNFLTCPNYDKETVFIKAKGTIGSGLRILDPNGYWNSTLRWPGSYSVSIQNVDDNNNTNVTDFAPKNQDCSREVKYTYGYKCGGDFSINRGGLTGNITKESNYSETISYQQPSYCTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 22 - protein, 310 aa, Artificial
  S. Aureus LukB variant
EIKSKITTVSEKNLDGDTKMYTRTATTSDTEKKISQSLQFNFLTCPNYDKETVFIKAKGTIGSGLKILNPNGYWNSTLRWPGSYSVSIQNVDDNNNSTNVTDFAPKNQDCSREVKYTYGYKCGGDFSINRGGLTGNITKEKNYSETISYQQPSYCTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTFNDKEKK

SEQ ID NO: 23 - protein, 22 aa, Artificial
  PelB signal sequence
MKYLLPTAAAGLLLLAAQPAMA

SEQ ID NO: 24 - protein, 11 aa, Artificial
  His-Tag
NSAHHHHHHGS

SEQ ID NO: 25 - protein, 324 aa, Staphylococcus aureus
  misc_feature (36)..(36): Xaa can be any naturally occurring amino acid
NSAHKDSQDQNKKEHVDKSQQKEKRNVTNKDKNSTXPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 26 - protein, 324 aa, Staphylococcus aureus
NSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 27 - protein, 324 aa, Staphylococcus aureus
NSANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKSNEKTRFEVTYTRNQDILKNKPGIHYGQPILEQNKDGQRFIVVYEVDWKNKTVKVVEKYSDQNKPYKEG

SEQ ID NO: 28 - protein, 323 aa, Staphylococcus aureus
NSAHKDSQDQNKKEHVDKSQQKEKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYSDNKSFREG

SEQ ID NO: 29 - protein, 323 aa, Staphylococcus aureus
NSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNPKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYTDNKSFREG

SEQ ID NO: 30 - protein, 323 aa, Staphylococcus aureus
NSAHKDSQDQNKKEHVDKSQQKEKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNPKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYSDNKSFREG

SEQ ID NO: 31 - protein, 324 aa, Staphylococcus aureus
NSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 32 - protein, 324 aa, Staphylococcus aureus
NSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQKYDTIAIGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 33 - protein, 324 aa, Staphylococcus aureus; identical to SEQ ID NO: 26

SEQ ID NO: 34 - protein, 324 aa, Staphylococcus aureus
NSANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKYSDQNKPYKEG

SEQ ID NO: 35 - protein, 324 aa, Staphylococcus aureus
NSANKDSQDQTKKEHVDKAQQKEKRNVNDKDKNTPGPDDIGKNGKVTKRTETVYDEKTNILQNLQFDFIDDPTYDKNVLLVKKQGSIHSNLKFESHKEENNSSWLKYPSEYHVDFQVKSNRKTEILDQLPKNKISTAKVDSTFSYNSGGKFDSVKGVGRTSSNSYSKTISYNQQNYDTIASGKNNNWHVHWSVVANDLKYGGEVKNRNDDFLFYRNTRLSTVENPELSFASKYRYPALVRSGFNPEFLTYLSNEKSNEKTQFEVTYTRNQDVLKNKPGIHYAPPILEKNKDGQRLIVTYEVDWKNKTVKVIDKYSDENKPYKEG

SEQ ID NO: 36 - protein, 324 aa, Staphylococcus aureus; identical to SEQ ID NO: 27

SEQ ID NO: 37 - protein, 324 aa, Staphylococcus aureus
NSANKDSQDQTKKEHVDKAQQKEKRNVNDKDKNTPGPDDIGKNGKVTKRTETVYDEKTNILQNLQFDFIDDPTYDKNILLVKKQGSIHSNLKFESHKEENNSSWLKYPSEYHVDFQVKRNRKTEILDQLPKNKISTAKVDSTFSYNSGGKFDSVKGVGRTSSNSYSKTISYNQQNYDTIASGKNNNWHVHWSVVANDLKYGGEVKNRNDEFLFYRTTRLSTVENPELSFASKYRYPALVRSGFNPEFLTYLSNEKSNEKTQFEVTYTRNQDILKNKPGIHYAPPILEKNKDGQRLIVTYEVDWKNKTVKVIDKYSDDNKPYKEG

SEQ ID NO: 38 - protein, 324 aa, Staphylococcus aureus
NSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPSILEKNKDGQRLIVTYEVDWKNKTVKVVDKYSDDNKPYKEG

SEQ ID NO: 39 - protein, 309 aa, Staphylococcus aureus
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 40 - protein, 310 aa, Staphylococcus aureus; identical to SEQ ID NO: 16

SEQ ID NO: 41 - protein, 261 aa, Staphylococcus aureus
DKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 42 - protein, 309 aa, Staphylococcus aureus
KINSEIKQVSEKNLDGETKMYTRTATTSDSQKNITQSLQFNFLTEKNYDKETVFIKAKGTIGSGLRILEPNGYWNSTLRWPGSYSVSIQNVDDNNNTNVTDFAPKNQDESREVKYTYGYKTGGDFSINQGGLTGNITKESNYSETISYQQPSYRTLIDQPTTNKGVAWKVEAHLINNMGHDHTRQLTNDSDDRVKSEIFSLTRNGNLWAKDNFTPKNKMPVTVSEGFNPEFLAVMSHDKKDEGKSKFVVHYKRSMDEFKIDWNKHGFWGYWSGENHVDKKEEKLSALYEVDWKTHNVKFIKVLNDKEKK

SEQ ID NO: 43 - protein, 310 aa, Staphylococcus aureus
EIKSKITTVSEKNLDGDTKMYTRTATTSDTEKKISQSLQFNFLTEPNYDKETVFIKAKGTIGSGLKILNPNGYWNSTLTWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 44 - protein, 309 aa, Staphylococcus aureus
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDEKEEKLSALYEVDWKTHNVKFVKVLNDNEKK

SEQ ID NO: 45 - protein, 310 aa, Staphylococcus aureus
EIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 46 - protein, 309 aa, Staphylococcus aureus
KINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHDVKFVKVLNDNEKK

SEQ ID NO: 47 - protein, 309 aa, Staphylococcus aureus
KINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHNVKFVKVLNDNEKK

SEQ ID NO: 48 - protein, 309 aa, Staphylococcus aureus
KINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLPGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHDVKFVKVLNDNEKK

SEQ ID NO: 49 - protein, 309 aa, Staphylococcus aureus
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHNVKFVKVLNDNEKK

SEQ ID NO: 50 - protein, 309 aa, Staphylococcus aureus; identical to SEQ ID NO: 15

SEQ ID NO: 51 - protein, 309 aa, Staphylococcus aureus; identical to SEQ ID NO: 15

SEQ ID NO: 52 - DNA, 966 bp, Staphylococcus aureus
gctcacaaagattctcaggatcaaaataagaaggagcacgtcgacaagtctcagcagaaagacaagcgtaatgttacaaacaaggacaaaaacagcactgctccagacgacattggaaaaaacggtaagattactaaacgcaccgaaacggtatatgacgaaaaaacgaacattttgcaaaacttgcagttcgatttcattgacgaccccacttatgacaagaatgtccttctggtgaagaagcagggcagcattcactcaaacttgaaatttgagtctcacaaggaggagaagaactccaattggctgaaatacccatcagagtaccacgttgattttcaagtgaaacgtaaccgcaaaacggaaattttggaccaattgccgaaaaacaagatctccaccgcgaaagtagactcaacattcagttactcttccggcggaaagttcgacagcactaaggggatcgggcgcacttcttccaattcgtactcgaaaacgatttcttacaatcagcagaattatgacactatcgcatctggtaaaaataataactggcacgtgcattggtcggtgattgctaatgatttaaagtatggaggtgaggtaaaaaatcgtaatgacgagctgctgttttaccgtaacactcgcatcgcaaccgttgaaaacccggaattgtcctttgcctcgaaataccgctaccctgcattagttcgttcaggctttaatcccgagtttttgacttatctttccaatgaaaaatcgaacgagaagactcagttcgaggttacgtacacccgcaatcaggacattttgaagaaccgtccgggaattcactatgcgcctcccatcttagagaagaataaggatggacaacgtttgatcgttacatatgaagttgactggaaaaataagaccgtaaaggttgtggataagtattcggatgataataagccctataaagaaggg

SEQ ID NO: 53 - DNA, 966 bp, Staphylococcus aureus
gcgaacaaagattctcaggaccagaccaaaaaggagcacgtagacaaggcccagcaaaaagagaagcgtaatgtgaacgacaaagataagaatactccggggccagatgatatcggcaagaacggtaaagtcacgaagcgtacagtgtctgagtatgacaaagaaacaaacatcctgcagaacttacaattcgactttattgatgatccaacttacgataagaatgtgttgctggttaagaaacaaggttcaatccattctaacttgaagttcgagtcacaccgtaacgaaacgaacgcgtcgtggttgaaatatccgtcagagtatcatgttgattttcaagtacaacgtaatcccaaaacggaaattttggaccaattacctaaaaataagattagcaccgccaaggttgactcaactttctcctactcattaggaggaaagttcgattcgacaaaagggatcgggcgtacatcttcgaatagctacagtaagagcattagctataaccagcagaactatgatacgattgcttcagggaaaaataacaaccgtcacgtacattggtcagtggttgcgaacgatcttaaatatggaaacgagattaagaatcgtaacgacgaatttttgttttaccgcaatacacgccttagtaccgtggaaaatcccgagctgtccttcgcgtcgaagtatcgctatccggcccttgtgcgttcgggtttcaatcccgagttcttaacatatatttccaatgagaaaactaacgacaagactcgcttcgaagtcacctacactcgcaaccaggacattctgaaaaacaagcctggaattcattacgggcaaccaattttagagcagaataaggatggacagcgctttattgtggtatatgaggtggactggaagaataagacagtaaaagttgtggaaaagtactctgaccagaataagccctataaagaagga

SEQ ID NO: 54 - DNA, 963 bp, Artificial
  CC8 LukA variant
cataaagattcgcaggatcaaaataagaaggagcatgttgacaagagccagcagaaagacaagcgcaatgttacaaacaaagataagaactctacagcgcccgatgacattggtaagaacggcaagataactaagcggacggaaaccgtgtatgacgagaaaactaacattctgcaaaatttgcaatttgactttatcgacgatccaacctatgacaagaatgtcttgcttgtcaaaatgcaaggttcgattcattcaaaccttaaatttgaatcccacaaagaggagaaaaactctaattggttaaagtatccttcagaatatcacatagatttccaggtaaagagaaaccgtaaaacggagatactggatcaactgcctaaaaacaagatctcgacagctaaggtggacgctacgttctcgtactcgtctggtgggaagttcgactcgaccaaaggcattgggcgtacatcatcaaatagctattcaaagactattagctataatcagcagaactatgatacgatagcttcgggtaagaataacaactggcacgttcattggtcgatcattgcaaatgacttgaagtatggcggagaggtaaagaatcgcaacgatgagctgttattctatcgcaatacgagaattgcgactgtagagaacccggaattgtcttttgcctccaaatatcggtacccggcattggtacgctctggtttcaatcctgagtttttaacttacctttccaacgaaaagagtaatgagaagacccaatttgaggttacctacacccgtaaccaggatattttgaagaatcggccgggcatccattatgccccaccaatcctggagaaaaataaagacggtcagcggcttattgtgacttacgaggtcgattggaaaaataagacggtcaaggtagtggacaaatattctgatgacaataaaccgtacaaagctggc

SEQ ID NO: 55 - DNA, 966 bp, Artificial
  CC45 LukA variant
gctaataaggactcccaggaccagacaaagaaggaacacgtcgacaaagcccagcaaaaagaaaaacgcaacgtaaacgataaggacaagaacaccccaggacccgatgatattgggaagaacggtaaagtcacaaaacgcacagtgagcgagtacgataaagaaacaaatatcctgcaaaatctgcaatttgacttcatcgatgaccctacctatgataagaatgtgttgttggttaagatgcagggaagtattcattccaacttgaaattcgagagccaccgtaacgaaacgaatgcgagttggttaaagtacccttcagaataccacattgattttcaggtgcagcgtaacccgaaaaccgaaatcttagaccagctgcctaaaaacaagatttctacggccaaggtggacgcaactttcagttatagtcttggaggaaagttcgacagtaccaaaggtatcggccgcacatcctcaaacagctattcgaaatccatttcttacaaccagcaaaattatgacacgatcgcctcaggtaagaacaacaatcgtcatgtgcattggagcatcgtggctaacgatttgaaatatggtaacgaaatcaaaaatcgcaatgacgagttcttgttttaccgcaatactcgcctttctacggtagagaatcctgagcttagctttgcgagcaagtatcgttaccctgctcttgtacgttcgggtttcaacccagagttccttacttatatctccaatgagaagacgaacgataaaacccgttttgaagttacatacacgcgtaatcaggacatcttaaagaataaaccggggattcattatgggcagccgatcttagagcaaaataaggatggacagcgtttcattgtagtgtatgaggttgactggaagaacaagacggtaaaagtagttgaaaagtattccgaccaaaacaagccttataaggcgggt

SEQ ID NO: 56 - DNA, 963 bp, Artificial
  CC8 LukA variant
cacaaagacagccaggatcaaaacaagaaagagcacgtggacaagagccagcaaaaggataaacgtaacgttaccaacaaggacaaaaacagcaccgcgccggacgatatcggcaagaacggcaaaattaccaagcgtaccgagaccgtgtacgatgaaaaaaccaacatcctgcagaacctgcaattcgactttattgacgatccgacctgcgataaaaacgtgctgctggttaagatgcagggcagcatccacagcaacctgaaattcgaaagccacaaagaggaaaagaacagcaactggctgaagtacccgagcgagtatcacattgactttcaggtgaaacgtaaccgtaagaccgaaatcctggatcaactgccgaagaacaaaattagcaccgcgaaggtttgcgcgaccttcagctacagcagcggttgcaaatttgacagcaccaagtgcatcggccgtaccagcagcaacagctatagcaaaaccatcagctacaaccagcaaaactatgataccattgcgagcggcaagaacaacaactggcacgtgcactggagcatcattgcgaacgacctgaaatacggtggcgaggttaagaaccgtaacgatgaactgctgttctatcgtaacacccgtatcgcgaccgtggagaacccggaactgagctttgcgagcaaataccgttatccggcgctggtgcgtagcggtttcaacccggagtttctggtttacctgagcaacgagaaaagcaacgaaaagacccagttcgaagttacctacacccgtaaccaagacatcctgaagaaccgtccgggtatccactatgctccgccgattctggagaagaacaaagatggccaacgtctgattgtgacctatgaagttgactggaagaacaaaaccgttaaagtggttgataagtacagcgacgataacaaaccgtataaggcgggt

SEQ ID NO: 57 - DNA, 966 bp, Artificial
  CC45 LukA variant
gcaaacaaagactcacaagatcagacaaagaaagagcatgtagacaaagctcaacagaaggaaaagcgcaatgtgaacgacaaggataaaaatactcctggtccagatgacattggtaagaatggtaaagttactaagcggaccgtctctgaatatgataaggagacaaatattctccagaatttgcaattcgatttcattgatgatccgacgtgcgataagaacgtattgctcgttaaaatgcagggctccatccattcgaatctcaagttcgaatcccatcgcaacgagacaaacgcttcctggctcaaatatcctagcgagtatcatatcgacttccaagttcaacggaaccctaaaactgaaatccttgatcaactccctaagaacaaaatctcaactgccaaggtctgtgccacattttcttattctcttggctgcaaattcgattcaacaaagtgtattggtcgtacatcaagtaatagctatagtaaaagcatcagttataaccagcaaaactatgatacaatcgcgtcaggcaaaaacaataatcgtcatgtccattggtccattgtcgcgaacgaccttaagtacggtaacgaaattaagaatcggaacgatgagtttttgttctatcgcaacacccgtctgtctactgtcgaaaacccggagttgtccttcgcaagtaaatatcgctatcctgctttggtacgttctgggtttaacccggaatttctcgtctacatcagcaacgagaaaacaaatgacaaaacgcgctttgaagtcacgtacacacgtaatcaggacatcttaaaaaataaaccagggattcactatggtcagccaatcttggagcagaataaagacggccagcgtttcattgtcgtttatgaagtggactggaaaaacaaaactgttaaggtggttgagaaatattccgaccaaaacaaaccgtataaggccggt

SEQ ID NO: 58 - DNA, 927 bp, Staphylococcus aureus
aaaatcaattctgaaattaagcaagtgtccgaaaaaaatttggatggagacacgaagatgtatacgcgtactgctacgacgtcagactcccagaagaacattacacagagtctgcaatttaattttctgacagaaccaaactatgacaaggaaactgtctttattaaggctaaagggactatcggaagcggcttacgcattttagaccccaacggttattggaatagcacgctgcgctggccgggcagttactcagtatcaatccaaaatgtcgatgataacaataacaccaatgttaccgatttcgcccccaagaaccaggatgaatcgcgcgaggttaaatacacatacggctacaagacaggcggtgactttagcatcaaccgtgggggcttgacagggaatattactaaggaatcaaattatagtgagactatctcttatcaacaaccgtcctatcgtaccttattagaccagagtacctcccacaaaggtgtagggtggaaagttgaagcgcacctgattaataatatgggtcacgatcacacacgccaactgaccaacgacagtgacaaccgcacaaaaagtgaaatttttagtcttacccgtaacggaaatctgtgggccaaagacaattttacaccgaaagataagatgccggtcactgtatctgaggggttcaatcccgagtttttagcagtaatgtcgcatgacaaaaaggacaaagggaaatcccagtttgttgtccactataagcgtagcatggatgaattcaaaatcgactggaaccgtcacggtttctggggttactggtcaggtgagaaccacgtagacaagaaagaggagaaactgagcgcattatatgaggttgattggaaaacgcacaatgtgaaatttgttaaagtcctgaatgacaacgagaaaaag

SEQ ID NO: 59 - DNA, 930 bp, Staphylococcus aureus
gaaattaagtctaagatcacaacagtatcggagaaaaacctggatggcgatactaagatgtatacacgcaccgccactacttcggacacggagaagaagatctcacaatcgttacagtttaattttcttacagaaccgaactacgacaaagagaccgtcttcattaaagctaaaggtacgattggttcgggattaaaaattctgaatccgaatggctattggaacagtaccttacgttggccggggtcatattctgtatccattcaaaacgtggacgacaataacaacagcaccaatgtgacagatttcgctccaaagaatcaggatgagtcccgcgaggtgaaatatacctatgggtacaaaacaggaggtgactttagcattaaccgtggtggcttgactggtaatatcacgaaggaaaaaaattactctgagactatttcctaccaacagccgtcgtatcgcaccttgatcgaccaaccaacgactaacaaaggggtcgcgtggaaagttgaggcccacagtattaacaatatgggccacgatcacactcgtcagcttactaacgattcggatgaccgcgtcaagtcggaaattttcagcctgacgcgtaacggaaatttgtgggctaaagacaatttcactcctaagaacaagatgcccgtgactgtttccgaaggctttaatcccgaattcttagcggtgatgtctcatgataaaaatgataaaggaaaatcgcgcttcattgtgcattataagcgttctatggacgacttcaaattggattggaataagcacggattctgggggtactggtccggggaaaatcacgtagatcaaaaggaagagaagttgtccgctttgtatgaagtggactggaagactcacgacgttaagttgatcaagaccttcaatgacaaagagaagaaa

SEQ ID NO: 60 - DNA, 927 bp, Artificial
  CC8 LukB Variant
aagatcaattcggaaattaaacaggtaagtgagaaaaatttggatggcgataccaaaatgtacacccgcaccgctaccacgtcagattcacaaaaaaatattacacagtccttgcagttcaatttcctgacagaaccgaattacgacaaggagactttgttcattaaagccaagggaaccatcgggtccggattgcgtatcttggacccgaacggatattggaactcgaccttacgttggccggggtcttacagtgttagtatccaaaacgtagatgataacaataacacaaacgtgacagattttgcacctaaaaaccaggacgaaagccgcgaggtaaagtacacatatgggtataaaacagggggggacttttccatcaaccgtggtggtttgaccgggaacatcaccaaagagtcaaattacagtgagaccatcagttatcagcagccgtcctatcgtacattattggatcagtcgacttcacataaaggggtcggatggaaagtagaggctcatttgatcaacaacatgggtcacgatcatacacgtcagttaacgaacgatagcgataatcgcacgaagtcagaaatctttagtctgactcgtaacggtaacttgtgggccaaggacaatttcacgcccaaagataagatgcctgtgacggtatcggaggggttcaatccagaattccttgctgtaatgtcccatgacaaaaaagacaagggcaaatcgcaatttgtagtccactataagcgttctatggacgagttcaagattgactggaaccgccacggcttctgggggtactggagtggtgagaatcatgtggataaaaaggaggagaaacttagcgccctgtatgaggtagattggaaaacacacaatgtcaagttcgtgaaagttcttaatgacaacgaaaaaaaa

SEQ ID NO: 61 - DNA, 930 bp, Artificial
  CC45 LukB Variant
gagatcaagagcaaaattaccaccgtgagcgaaaagaacctggacggtgataccaaaatgtatacccgtaccgcgaccaccagcgacaccgagaagaaaattagccagagcctgcaattcaactttctgaccgagccgaactacgataaggaaaccctgttcatcaaggcgaaaggcaccattggtagcggcctgaaaatcctgaacccgaacggttattggaacagcaccctgcgttggccgggtagctacagcgtgagcatccagaacgttgacgataacaacaacagcaccaacgtgaccgacttcgcgccgaagaaccaagatgagagccgtgaagttaaatacacctatggttacaaaaccggtggcgactttagcattaaccgtggtggcctgaccggcaacatcaccaaggagaaaaactatagcgaaaccattagctatcagcaaccgagctaccgtaccctgatcgatcagccgaccaccaacaagggtgtggcgtggaaagttgaggcgcacagcattaacaacatgggccacgaccacacccgtcaactgaccaacgatagcgacgatcgtgtgaagagcgaaatcttcagcctgacccgtaacggtaacctgtgggcgaaagacaactttaccccgaagaacaaaatgccggtgaccgttagcgagggtttcaacccggaatttctggcggtgatgagccacgacaagaacgataagggcaaaagccgtttcattgttcactacaaacgtagcatggacgatttcaagctggactggaacaaacacggtttttggggctattggagcggcgagaaccacgttgatcagaaagaggagaaactgagcgcgctgtacgaagtggactggaagacccacgatgttaagctgatcaaaacctttaacgataaagaaaagaaa

SEQ ID NO: 62 - DNA, 936 bp, Artificial
  CC8 LukA variant
gctcacaaagattctcaggatcaaaataagaaggagcacgtcgacaagtctcagcagaaagacaagcgtaatgttacaaacaaggacaaaaacagcactgctccagacgacattggaaaaaacggtaagattactaaacgcaccgaaacggtatatgacgaaaaaacgaacattttgcaaaacttgcagttcgatttcattgacgaccccacttatgacaagaatgtccttctggtgaagaagcagggcagcattcactcaaacttgaaatttgagtctcacaaggaggagaagaactccaattggctgaaatacccatcagagtaccacgttgattttcaagtgaaacgtaaccgcaaaacggaaattttggaccaattgccgaaaaacaagatctccaccgcgaaagtagactcaacattcagttactcttccggcggaaagttcgacagcactaaggggatcgggcgcacttcttccaattcgtactcgaaaacgatttcttacaatcagcagaattatgacactatcgcatctggtaaaaataataactggcacgtgcattggtcggtgattgctaatgatttaaagtatggaggtgaggtaaaaaatcgtaatgacgagctgctgttttaccgtaacactcgcatcgcaaccgttgaaaacccggaattgtcctttgcctcgaaataccgctaccctgcattagttcgttcaggctttaatcccgagtttttgacttatctttccaatgaaaaatcgaacgagaagactcagttcgaggttacgtacacccgcaatcaggacattttgaagaaccgtccgggaattcactatgcgcctcccatcttagagaagaataaggatggacaacgtttgatcgttacatatgaagttgactggaaaaataagaccgtaaaggttgtggataagtat

SEQ ID NO: 63 - DNA, 936 bp, Artificial
  CC45 LukA variant
gcaaataaagactctcaagatcagactaaaaaggaacatgttgataaggcgcaacaaaaagaaaagcgtaatgtcaatgataaggacaagaatactccgggacccgacgacattggcaagaacggaaaggtgacaaagcgtaccgttagtgagtacgacaaggaaacaaatatcctgcagaacttacagttcgattttattgacgatcctacctatgacaagaatgtcctgttggtgaagaaacagggcagcattcattccaatttaaaatttgaaagccatcgtaacgaaacaaatgcatcttggcttaaatacccttctgagtaccacgtagattttcaggtacaacgcaacccaaaaaccgaaattctggatcaactgcccaagaataaaatttctacggctaaagttgacagtacatttagctacagtttagggggaaagtttgatagtacaaaaggaattggtcgtacttccagtaactcctattcgaaatctatttcctataatcaacagaattacgacaccatcgcatccggtaaaaacaataatcgccacgtacattggagtgttgtcgcgaatgacttaaagtacggtaacgaaatcaagaaccgcaacgacgaattcttattctatcgtaacacgcgtttaagcaccgtcgagaaccccgagttatcctttgctagcaaatatcgctatcctgcgttagtacgctcagggttcaatcctgagttcttaacctacatctccaacgagaaaactaatgataagacacgcttcgaggtgacctacacccgtaatcaggatatccttaaaaataaaccgggtattcattacgggcaacccattttagaacagaataaggacggccaacgttttatcgtggtctatgaggttgattggaagaacaagacagtgaaagtggttgaaaagtat

SEQ ID NO: 64 - protein, 311 aa, Artificial
  LukA variant
HKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKY

SEQ ID NO: 65 - protein, 312 aa, Artificial
  LukA variant
ANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKY